(19) United States
(12) Patent Application Publication
(10) Pub. No.: US 2009/0253908 A1
(43) Pub. Date: Oct. 8, 2009

(54) NOVEL M3 MUSCARINIC ACETYLCHOINE RECEPTOR ANTAGONISTS

(75) Inventors: Brian W. Budzik, Collegeville, PA (US); Anthony W. J. Cooper, Hertfordshire (GB); David Francis Corbett, Essex (GB); Jian Jin, Collegeville, PA (US); Dramane I. Laine, King of Prussia, PA (US); Yonghui Wang, Collegeville, PA (US); Michael Lee Moore, Collegeville, PA (US); Ralph A. Rivero, Collegeville, PA (US); Dongchuan Shi, Collegeville, PA (US); Feng Wang, Collegeville, PA (US); Haibo Xie, King of Prussia, PA (US); Chongjie Zhu, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXO GROUP LIMITED

(21) Appl. No.: 10/598,743

(22) PCT Filed: Mar. 11, 2005
(86) PCT No.: PCT/US05/08302

§ 371 Date: Sep. 11, 2006

Related U.S. Application Data
(60) Provisional application No. 60/552,106, filed on Mar. 11, 2004.

(51) Int. Cl. C07F 5/02; C07D 241/04

(52) U.S. Cl. 544/229; 544/389

(57) ABSTRACT

Muscarinic Acetylcholine receptor antagonists and methods of using them are provided.

### FIELD OF THE INVENTION

This invention relates to novel derivatives of biaryl amines, pharmaceutical compositions, processes for their preparation, and use thereof in treating M₃ muscarinic acetylcholine receptor mediated diseases.

### BACKGROUND OF THE INVENTION

Acetylcholine released from cholinergic neurons in the peripheral and central nervous systems affects many different biological processes through interaction with two major classes of acetylcholine receptors—the nicotinic and the muscarinic acetylcholine receptors. Muscarinic acetylcholine receptors (mAChRs) belong to the superfamily of G-protein coupled receptors that have seven transmembrane domains. There are five subtypes of mAChRs, termed M1-M5, and each is the product of a distinct gene. Each of these five subtypes displays unique pharmacological properties. Muscarinic acetylcholine receptors are widely distributed in vertebrate organs where they mediate many of the vital functions. Muscarinic receptors can mediate both inhibitory and excitatory actions. For example, in smooth muscle found in the airways, M3 mAChRs mediate contractile responses. For review, please see Caulfield (1993 Pharmac. Ther. 58:319-79).

In the lungs, mAChRs have been localized to smooth muscle in the trachea and bronchi, the submucosal glands, and the parasympathetic ganglia. Muscarinic receptor density is greatest in parasympathetic ganglia and then decreases in density from the submucosal glands to tracheal and then bronchial smooth muscle. Muscarinic receptors are nearly absent from the alveoli. For review of mAChR expression and function in the lungs, please see Fryer and Jacoby (1998 Am J Respir Crit. Care Med 158(5, pt 3) S 154-60).

Three subtypes of mAChRs have been identified as important in the lungs, M1, M2 and M3 mAChRs. The M3 mAChRs, located on airway smooth muscle, mediate muscle contraction. Stimulation of M3 mAChRs activates the enzyme phospholipase C via binding of the stimulatory G protein Gq/11 (Gs), leading to liberation of phosphatidyl inositol-4,5-bisphosphate, resulting in phosphorylation of contractile proteins. M3 mAChRs are also found on pulmonary submucosal glands. Stimulation of this population of M3 mAChRs results in mucus secretion.

M2 mAChRs make up approximately 50-80% of the cholinergic receptor population on airway smooth muscles. Although the precise function is still unknown, they inhibit catecholaminergic relaxation of airway smooth muscle via inhibition of cAMP generation. Neuronal M2 mAChRs are located on postganglionic parasympathetic nerves. Under normal physiologic conditions, neuronal M2 mAChRs provide tight control of acetylcholine release from parasympathetic nerves. Inhibitory M2 mAChRs have also been demonstrated on sympathetic nerves in the lungs of some species. These receptors inhibit release of noradrenaline, thus decreasing sympathetic input to the lungs.

M1 mAChRs are found in the pulmonary parasympathetic ganglia where they function to enhance neurotransmission. These receptors have also been localized to the peripheral lung parenchyma, however their function in the parenchyma is unknown.

Muscarinic acetylcholine receptor dysfunction in the lungs has been noted in a variety of different pathophysiological states. In particular, in asthma and chronic obstructive pulmonary disease (COPD), inflammatory conditions lead to loss of inhibitory M2 muscarinic acetylcholine autoreceptor function on parasympathetic nerves supplying the pulmonary smooth muscle, causing increased acetylcholine release following vagal nerve stimulation (Fryer et al. 1999 Life Sci 64 (6-7) 449-55). This mAChR dysfunction results in airway hyperreactivity and hyperresponsiveness mediated by increased stimulation of M3 mAChRs. Thus the identification of potent mAChR antagonists would be useful as therapeutics in these mAChR-mediated disease states.

COPD is an imprecise term that encompasses a variety of progressive health problems including chronic bronchitis, chronic bronchiolitis and emphysema, and it is a major cause of mortality and morbidity in the world. Smoking is the major risk factor for the development of COPD; nearly 50 million people in the U.S. alone smoke cigarettes, and an estimated 3,000 people take up the habit daily. As a result, COPD is expected to rank among the top five as a world-wide health burden by the year 2020. Inhaled anti-cholinergic therapy is currently considered the “gold standard” as first line therapy for COPD (Pauwels et al. 2001 Am. J. Respir. Crit. Care Med. 163:1256-1276).

Despite the large body of evidence supporting the use of anti-cholinergic therapy for the treatment of airway hyperreactive diseases, relatively few anti-cholinergic compounds are available for use in the clinic for pulmonary indications. More specifically, in United States, Ipratropium Bromide (Atrovent©; and Combivent©, in combination with albuterol) is currently the only inhaled anti-cholinergic marketed for the treatment of airway hyperreactive diseases. While this compound is a potent anti-muscarinic agent, it is short acting, and thus must be administered as many as four times daily in order to provide relief for the COPD patient. In Europe and Asia, the long-acting anti-cholinergic Tiotropium Bromide (Spiriva©) was recently approved, however this product is currently not available in the United States. Thus, there remains a need for novel compounds that are capable of causing blockade at mAChRs which are long acting and can be administered once-daily for the treatment of airway hyperreactive diseases such as asthma and COPD.

Since mAChRs are widely distributed throughout the body, the ability to apply anti-cholinergics locally and/or topically to the respiratory tract is particularly advantageous, as it would allow for lower doses of the drug to be utilized. Furthermore, the ability to design topically active drugs that have long duration of action, and in particular, are retained either at the receptor or by the lung, would allow the avoidance of unwanted side effects that may be seen with systemic anti-cholinergic use.

### SUMMARY OF THE INVENTION

This invention provides for a method of treating a muscarinic acetylcholine receptor (mAChR) mediated disease, wherein acetylcholine binds to an M₃ mAChR and which method comprises administering an effective amount of a compound of Formula (I) or a pharmaceutically acceptable salt thereof.

This invention also relates to a method of inhibiting the binding of acetylcholine to its receptors in a mammal in need thereof which comprises administering to aforementioned mammal an effective amount of a compound of Formula (I).

The present invention also provides for the novel compounds of Formula (I), and pharmaceutical compositions comprising a compound of Formula (I), and a pharmaceutical carrier or diluent.

Compounds of Formula (I) useful in the present invention are represented by the structure:

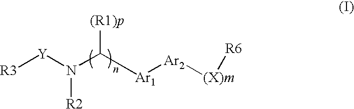

wherein

Ar1 and Ar2, are independently, selected from the group consisting of optionally substituted phenyl and optionally substituted monocyclic heteroaryl;

R6 is NR₇R₈, or an optionally substituted saturated or partially unsaturated 4-10 membered ring system in which one or more rings contain one or more secondary or tertiary nitrogens, and optionally contain one or more O, or S;

X is C(R1)p, or C(O); wherein, when X is C(R1)p, m is an interger from 0 to 3; when X is C(O), m is 1;

p is an interger from 0 to 2;

n is an interger from 0 to 3;

Y is C(O), S(O)q, HNC(O), or OC(O); wherein, q is 1 or 2;

R1 and R2 are independently selected from the group consisting of hydrogen, optionally substituted C₁-C₁₀ alkyl, optionally substituted C₃-C₁₀ cycloalkyl, optionally substituted C₃-C₁₀ cycloalkyl alkyl, optionally substituted heterocylic, optionally substituted heterocyclicalkyl, optionally substituted alkenyl, optionally substituted aryl, optionally substituted aryl alkyl, optionally substituted heteroaryl, and optionally substituted heteroaryl alkyl;

R3 is selected from the group consisting of optionally substituted aryl, optionally substituted heteroaryl, optionally substituted alkenyl, optionally substituted C₁-C₁₀ alkyl, optionally substituted C₃-C₁₀ cycloalkyl, optionally substituted C₃-C₁₀ cycloalkyl alkyl, optionally substituted aryl alkyl, and optionally substituted heteroaryl alkyl; wherein, when substituted, a group is substituted by one or more radicals selected from the group consisting of halogen, cyano, hydroxy, hydroxy substituted C₁₋₁₀ alkyl, C₁₋₁₀ alkoxy, S(O)_{m′}C₁₋₁₀ alkyl, C(O)R4, C(O)NR₄R₅; C(O)OH; S(O)₂NR₄R₅, NHC(O)R₄, NHS(O)₂R₄, C₁₋₁₀ alkyl, alkenyl, halosubstituted C₁₋₁₀ alkyl, optionally substituted aryl, optionally substituted arylalkyl, optionally substituted heteroaryl, optionally substituted heteroaryl alkyl, wherein these aryl or heteroaryl moieties may be substituted one to two times by halogen, hydroxy, hydroxy substituted alkyl, C₁₋₁₀ alkoxy, S(O)_{m′}C₁₋₁₀ alkyl, C₁₋₁₀ alkyl, or halosubstituted C₁₋₁₀ alkyl; and m′ is 0, 1, or 2;

R₄ and R₅, are independently, selected from the group consisting of hydrogen, optionally substituted C₁₋₁₀ alkyl, optionally substituted alkenyl, optionally substituted C₃-C₁₀ cycloalkyl, optionally substituted C₃-C₁₀ cycloalkyl alkyl, optionally substituted aryl, optionally substituted aryl alkyl, optionally substituted heteroaryl, and optionally substituted heteroaryl alkyl; or R₄ and R₅ together with the nitrogen to which they are attached form a 5 to 7 member ring which may optionally comprise an additional heteroatom selected from O, and S;

R₇ and R₈, are independently, selected from the group consisting of hydrogen, optionally substituted C₁₋₁₀ alkyl, optionally substituted alkenyl, optionally substituted C₃-C₁₀ cycloalkyl, optionally substituted C₃-C₁₀ cycloalkyl alkyl, optionally substituted aryl, optionally substituted arylalkyl, optionally substituted heteroaryl, optionally substituted heteroarylalkyl, optionally substituted heterocyclic, and optionally substituted heterocyclicalkyl; or R₇ and R₈ together with the nitrogen to which they are attached form a 5 to 7 member ring which may optionally comprise an additional heteroatom selected from O, N and S;

or a pharmaceutically acceptable salt thereof.

### DETAILED DESCRIPTION

The present invention includes all hydrates, solvates, complexes and prodrugs of the compounds of this invention. Prodrugs are any covalently bonded compounds that release the active parent drug according to Formula I in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any subformula thereof is independent of its meaning, or any other substituent's meaning, at any other occurrence, unless specified otherwise.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

For use herein the term “the aryl, heteroaryl, and heterocyclic containing moieties” refers to both the ring and the alkyl, or if included, the alkenyl rings, such as aryl, arylalkyl, and aryl alkenyl rings. The term “moieties” and “rings” may be interchangeably used throughout.

As used herein, “optionally substituted” unless specifically defined shall mean such groups as hydrogen; halogen, such as fluorine, chlorine, bromine or iodine; cyano; hydroxy; hydroxy substituted C₁₋₁₀ alkyl; C₁₋₁₀ alkoxy, such as methoxy or ethoxy; S(O)_{m′}C₁₋₁₀ alkyl, wherein m′ is 0, 1 or 2, such as methyl thio, methyl sulfinyl or methyl sulfonyl; amino, mono & di-substituted amino, such as in the NR₇R₈ group; NHC(O)R₇; C(O)NR₇R₈; C(O)R7; C(O)OH; S(O)₂NR₇R₈; NHS(O)₂R₇, C₁₋₁₀ alkyl, such as methyl, ethyl, propyl, isopropyl, or t-butyl; alkenyl, such as ethenyl, 1-propenyl, 2-propenyl, or 2-methyl-1-propenyl; halosubstituted C₁₋₁₀ alkyl, such CF₃; an optionally substituted aryl, such as phenyl, or an optionally substituted arylalkyl, such as benzyl or phenethyl, optionally substituted heterocylic, optionally substituted heterocyclic alkyl, optionally substituted heteroaryl, optionally substituted heteroaryl alkyl, wherein these aryl, heteroaryl, or heterocyclic moieties may be substituted one to two times by halogen; hydroxy; hydroxy substituted alkyl; C₁₋₁₀ alkoxy; S(O)_{m′}C₁₋₁₀ alkyl; amino, mono & di-substituted alkyl amino, such as in the NR₇R₈ group; C₁₋₁₀ alkyl, or halosubstituted C₁₋₁₀ alkyl, such as CF₃.

Suitable pharmaceutically acceptable salts are well known to those skilled in the art and include basic salts of inorganic and organic acids, such as hydrochloric acid, hydrobromic acid, sulphuric acid, phosphoric acid, methane sulphonic acid, ethane sulphonic acid, acetic acid, trifluoroacetic acid, malic acid, tartaric acid, citric acid, lactic acid, oxalic acid, succinic acid, fumaric acid, maleic acid, benzoic acid, salicylic acid, phenylacetic acid and mandelic acid.

The following terms, as used herein, refer to:
“halo” or “halogen”—chloro, fluoro, bromo and iodo.
“C₁₋₁₀alkyl” or “alkyl”—both straight and branched chain moieties of 1 to 10 carbon atoms, unless the chain length is otherwise limited, including, but not limited to, methyl, ethyl, n-propyl, iso-propyl, n-butyl, sec-butyl, iso-butyl, tert-butyl, n-pentyl and the like.
“C₁-C₁₀ alkoxy” includes straight and branched chain radicals of the likes of —O—CH₃, —O—CH₂CH₃, and the n-propoxy, isopropoxy, n-butoxy, sec-butoxy, isobutoxy, tert-butoxy, pentoxy, and hexoxy, and the like.
“C₃-C₁₀ cycloalkyl” is used herein to mean cyclic moiety, including but not limited to cyclopropyl, cyclopentyl, cyclohexyl, and the like.
“alkenyl” is used herein at all occurrences to mean straight or branched chain moiety of 2-10 carbon atoms, unless the chain length is limited thereto, including, but not limited to ethenyl, 1-propenyl, 2-propenyl, 2-methyl-1-propenyl, 1-butenyl, 2-butenyl and the like.
“aryl”—phenyl and naphthyl;
“heteroaryl” (on its own or in any combination, such as “heteroaryloxy”, or “heteroaryl alkyl”)—a 5-10 membered aromatic ring system in which one or more rings contain one or more heteroatoms selected from the group consisting of N, O or S, such as, but not limited, to pyrrole, pyrazole, furan, thiophene, quinoline, isoquinoline, quinazolinyl, pyridine, pyrimidine, oxazole, tetrazole, thiazole, thiadiazole, triazole, imidazole, or benzimidazole.
“heterocyclic” (on its own or in any combination, such as “heterocyclicalkyl”)—a saturated or partially unsaturated 4-10 membered ring system in which one or more rings contain one or more heteroatoms selected from the group consisting of N, O, or S; such as, but not limited to, pyrrolidine, piperidine, piperazine, morpholine, tetrahydropyran, thiomorpholine, or imidazolidine. Furthermore, sulfur may be optionally oxidized to the sulfone or the sulfoxide.
“secondary nitrogen” is used herein to mean a nitrogen directly connected to one hydrogen, one optionally substituted carbon, and one optionally substituted carbon, C(O), or S(O)m′; where in m′ is 1 or 2.
“tertiary nitrogen” is used herein to mean a nitrogen directly connected to two independent optionally substituted carbons, and one optionally substituted carbon, C(O), or S(O)m′; where in m′ is 1 or 2.
“quaternary ammonium nitrogen” is used herein to mean a nitrogen directly connected to four independent optionally substituted carbons.
“arylalkyl” or “heteroarylalkyl” or “heterocyclicalkyl” is used herein to mean C₁₋₁₀ alkyl, as defined above, attached to an aryl, heteroaryl or heterocyclic moiety, as also defined herein, unless otherwise indicated.
“sulfinyl”—the oxide S(O) of the corresponding sulfide, the term “thio” refers to the sulfide, and the term “sulfonyl” refers to the fully oxidized S(O)₂ moiety.

The preferred compounds of Formula I include those compounds wherein:

Ar1 and Ar2, are independently, selected from the group consisting of optionally substituted phenyl and optionally substituted monocyclic heteroaryl;

R6 is an optionally substituted saturated or partially unsaturated 4-10 membered ring system in which one or more rings contain one or more secondary or tertiary nitrogens;

X is C(R1)p, m is an interger from 0 to 3;

p is 2;

n is an interger from 1 to 3;

Y is C(O), or S(O)q; wherein, q is 1 or 2;

R1 is hydrogen

R2 is selected from the group consisting of hydrogen, optionally substituted C₁-C₁₀ alkyl, optionally substituted alkenyl, optionally substituted C₃-C₁₀ cycloalkyl, optionally substituted C₃-C₁₀ cycloalkyl alkyl, optionally substituted heterocylic, optionally substituted heterocyclicalkyl, optionally substituted aryl, optionally substituted aryl alkyl, optionally substituted heteroaryl, and optionally substituted heteroaryl alkyl;

R3 is selected from the group consisting of optionally substituted aryl, optionally substituted heteroaryl, optionally substituted alkenyl, optionally substituted C₁-C₁₀ alkyl, optionally substituted C₃-C₁₀ cycloalkyl, and optionally substituted C₃-C₁₀ cycloalkyl alkyl; wherein, when substituted, a group is substituted by one or more radicals selected from the group consisting of halogen, cyano, hydroxy, hydroxy substituted C₁₋₁₀ alkyl, C₁₋₁₀ alkoxy, S(O)_{m′}C₁₋₁₀ alkyl, C(O)R4, C(O)NR₄R₅; C(O)OH; S(O)₂NR₄R₅, NHC(O)R₄, NHS(O)₂R₄, C₁₋₁₀ alkyl, alkenyl, halosubstituted C₁₋₁₀ alkyl, optionally substituted aryl, optionally substituted arylalkyl, optionally substituted heteroaryl, optionally substituted heteroaryl alkyl, wherein these aryl or heteroaryl moieties may be substituted one to two times by halogen, hydroxy, hydroxy substituted alkyl, C₁₋₁₀ alkoxy, S(O)_{m′}C₁₋₁₀ alkyl, C₁₋₁₀ alkyl, or halosubstituted C₁₋₁₀ alkyl; and m′ is 0, 1, or 2;

R₄ and R₅, are independently, selected from the group consisting of hydrogen, optionally substituted C₁₋₁₀ alkyl, optionally substituted alkenyl, optionally substituted C₃-C₁₀ cycloalkyl, optionally substituted C₃-C₁₀ cycloalkyl alkyl, optionally substituted aryl, optionally substituted aryl alkyl, optionally substituted heteroaryl, and optionally substituted heteroaryl alkyl; or R₄ and R₅ together with the nitrogen to which they are attached form a 5 to 7 member ring which may optionally comprise an additional heteroatom selected from O, and S;

R₇ and R₈, are independently, selected from the group consisting of hydrogen, optionally substituted C₁₋₁₀ alkyl, optionally substituted alkenyl, optionally substituted C₃-C₁₀ cycloalkyl, optionally substituted C₃-C₁₀ cycloalkyl alkyl, optionally substituted aryl, optionally substituted arylalkyl, optionally substituted heteroaryl, optionally substituted heteroarylalkyl, optionally substituted heterocyclic, and optionally substituted heterocyclicalkyl; or R₇ and R₈ together with the nitrogen to which they are attached form a 5 to 7 member ring which may optionally comprise an additional heteroatom selected from O, N and S;

or a pharmaceutically acceptable salt thereof.

Even more preferred are those compounds where:

Ar1 and Ar2, are independently, selected from the group consisting of optionally substituted phenyl and optionally substituted monocyclic heteroaryl;

R6 is an optionally substituted saturated or partially unsaturated 5-8 membered ring system in which one or more rings contain one or more secondary or tertiary nitrogens;

X is C(R1)p;

R1 is hydrogen

p is 2;

m is 1;

n is 1;

Y is C(O), or S(O)_{q}; wherein, q is 1 or 2;

R2 is selected from the group consisting of hydrogen, optionally substituted C₁-C₁₀ alkyl, optionally substituted alkenyl, optionally substituted C₃-C₁₀ cycloalkyl, optionally substituted C₃-C₁₀ cycloalkyl alkyl, optionally substituted heterocylic, optionally substituted heterocyclicalkyl, optionally substituted aryl alkyl, and optionally substituted heteroaryl alkyl;

R3 is selected from the group consisting of optionally substituted aryl, optionally substituted heteroaryl, optionally substituted alkenyl, optionally substituted C₁-C₁₀ alkyl, optionally substituted C₃-C₁₀ cycloalkyl, and optionally substituted C₃-C₁₀ cycloalkyl alkyl; wherein, when substituted, a group is substituted by one or more radicals selected from the group consisting of halogen, cyano, hydroxy, hydroxy substituted C₁₋₁₀alkyl, C₁₋₁₀ alkoxy, S(O)_{m′}C₁₋₁₀ alkyl, C(O)R4, C(O)NR₄R₅; C(O)OH; S(O)₂NR₄R₅, NHC(O)R₄, NHS(O)₂R₄, C₁₋₁₀ alkyl, alkenyl, and halosubstituted C₁₋₁₀ alkyl; wherein m′ is 0, 1, or 2;

R₄ and R₅, are independently, selected from the group consisting of hydrogen, optionally substituted C₁₋₁₀ alkyl, optionally substituted alkenyl, optionally substituted C₃-C₁₀ cycloalkyl, optionally substituted C₃-C₁₀ cycloalkyl alkyl, optionally substituted aryl, optionally substituted aryl alkyl, optionally substituted heteroaryl, and optionally substituted heteroaryl alkyl; or R₄ and R₅ together with the nitrogen to which they are attached form a 5 to 7 member ring which may optionally comprise an additional heteroatom selected from O, and S;

R₇ and R₈, are independently, selected from the group consisting of hydrogen, optionally substituted C₁₋₁₀ alkyl, optionally substituted alkenyl, optionally substituted C₃-C₁₀ cycloalkyl, optionally substituted C₃-C₁₀ cycloalkyl alkyl, optionally substituted aryl, optionally substituted arylalkyl, optionally substituted heteroaryl, optionally substituted heteroarylalkyl, optionally substituted heterocyclic, and optionally substituted heterocyclicalkyl; or R₇ and R₈ together with the nitrogen to which they are attached form a 5 to 7 member ring which may optionally comprise an additional heteroatom selected from O, N and S;

or a pharmaceutically acceptable salt thereof.

The preferred compounds are selected from the group consisting of:
N-[(6-fluoro-3′-{[(3S)-3-methyl-1-piperazinyl]methyl}-3-biphenylyl)methyl]-3-oxo-2,3-dihydro-1H-indene-5-carboxamide bis(trifluoroacetate);
N-[(6-fluoro-3′-{[(3S)-3-methyl-1-piperazinyl]methyl}-3-biphenylyl)methyl]-3-propanoylbenzamide bis(trifluoroacetate);
3-acetyl-N-[(6-fluoro-3′-{[(3S)-3-methyl-1-piperazinyl]methyl}-3-biphenylyl)methyl]benzamide bis(trifluoroacetate);
N-[(6-fluoro-3′-{[(3S)-3-methyl-1-piperazinyl]methyl}-3-biphenylyl)methyl]-3-(2-oxopropyl)benzamide bis(trifluoroacetate);
3-(ethyloxy)-N-({6-fluoro-3′-[(3-methyl-1-piperazinyl)methyl]-3-biphenylyl}methyl)benzamide bis(trifluoroacetate);
3-acetyl-N-[(3′-{[(3S)-3-methyl-1-piperazinyl]methyl}-3-biphenylyl)methyl]benzamide bis(trifluoroacetate);
3-cyano-N-[(6-fluoro-3′-{[(3S)-3-methyl-1-piperazinyl]methyl}-3-biphenylyl)methyl]benzamide bis(trifluoroacetate);
3-acetyl-N-[(6-(methyloxy)-3′-{[(3S)-3-methyl-1-piperazinyl]methyl}-3-biphenylyl)methyl]benzamide bis(trifluoroacetate);
3-cyano-N-[(3′-{[(3S)-3-methyl-1-piperazinyl]methyl}-3-biphenylyl)methyl]benzamide bis(trifluoroacetate);
N-({3′-[(1S,4S)-2,5-diazabicyclo[2.2.1]hept-2-ylmethyl]-6-fluoro-3-biphenylyl}methyl)-1,3-benzodioxole-5-carboxamide bis(trifluoroacetate);
3-(ethyloxy)-N-({6-(methyloxy)-3′-[(3-methyl-1-piperazinyl)methyl]-3-biphenylyl}methyl)benzamide bis(trifluoroacetate);
N-[(3′-{[(3S)-3-methyl-1-piperazinyl]methyl}-3-biphenylyl)methyl]-1,3-benzodioxole-5-carboxamide bis(trifluoroacetate);
3-acetyl-N-({3′-[(3-methyl-1-piperazinyl)methyl]-3-biphenylyl}methyl)benzamide bis(trifluoroacetate);
3-acetyl-N-[(3′-{[(3R)-3-methyl-1-piperazinyl]methyl}-3-biphenylyl)methyl]benzamide bis(trifluoroacetate);
N-{[6-fluoro-3′-(1-piperazinylmethyl)-3-biphenylyl]methyl}-1,3-benzodioxole-5-carboxamide bis(trifluoroacetate);
methyl 3-{[({3′-[(3-methyl-1-piperazinyl)methyl]-3-biphenylyl}methyl)amino]carbonyl}benzoate bis(trifluoroacetate);
3-cyano-N-({3′-[(1S,4S)-2,5-diazabicyclo[2.2.1]hept-2-ylmethyl]-6-fluoro-3-biphenylyl}methyl)benzamide bis(trifluoroacetate);
3-cyano-N-({3′-[(3-methyl-1-piperazinyl)methyl]-3-biphenylyl}methyl)benzamide bis(trifluoroacetate);
3-acetyl-N-{[3′-(1-piperazinylmethyl)-3-biphenylyl]methyl}benzamide bis(trifluoroacetate);
3-cyano-N-[(6-(methyloxy)-3′-{[(3S)-3-methyl-1-piperazinyl]methyl}-3-biphenylyl)methyl]benzamide bis(trifluoroacetate);
3-(ethyloxy)-N-{[3′-(1-piperazinylmethyl)-3-biphenylyl]methyl}benzamide bis(trifluoroacetate);
3-cyano-N-{[6-fluoro-4′-(methyloxy)-3′-(1-piperazinylmethyl)-3-biphenylyl]methyl}benzamide bis(trifluoroacetate);
N-({3′-[(3-methyl-1-piperazinyl)methyl]-3-biphenylyl}methyl)-1,3-benzodioxole-5-carboxamide bis(trifluoroacetate);
N-{[6-fluoro-4′-(methyloxy)-3′-(1-piperazinylmethyl)-3-biphenylyl]methyl}-1,3-benzodioxole-5-carboxamide bis(trifluoroacetate);
3-(ethyloxy)-N-({3′-[(3-methyl-1-piperazinyl)methyl]-3-biphenylyl}methyl)benzamide bis(trifluoroacetate);
3-acetyl-N-({3′-[(1S,4S)-2,5-diazabicyclo[2.2.1]hept-2-ylmethyl]-3-biphenylyl}methyl)benzamide bis(trifluoroacetate);
N-({3′-[(3-methyl-1-piperazinyl)methyl]-3-biphenylyl}methyl)-2,1,3-benzoxadiazole-5-carboxamide bis(trifluoroacetate);
N-{[3′-(1-piperazinylmethyl)-3-biphenylyl]methyl}-2,1,3-benzoxadiazole-5-carboxamide bis(trifluoroacetate);
N-{[4′,6-difluoro-3′-(1-piperazinylmethyl)-3-biphenylyl]methyl}-1,3-benzodioxole-5-carboxamide bis(trifluoroacetate);
3-(methyloxy)-N-({3′-[(3-methyl-1-piperazinyl)methyl]-3-biphenylyl}methyl)benzamide bis(trifluoroacetate);
3-cyano-N-{[4′,6-difluoro-3′-(1-piperazinylmethyl)-3-biphenylyl]methyl}benzamide bis(trifluoroacetate);
3-cyano-N-{[6-(methyloxy)-3′-(1-piperazinylmethyl)-3-biphenylyl]methyl}benzamide bis(trifluoroacetate);
methyl 3-[({[3′-(1-piperazinylmethyl)-3-biphenylyl]methyl}amino)carbonyl]benzoate bis(trifluoroacetate);
3-(methylsulfonyl)-N-{[3′-(1-piperazinylmethyl)-3-biphenylyl]methyl}benzamide bis(trifluoroacetate);
N-[3-(4-methyl-1-piperazinyl)propyl]-N-{[3′-(1-piperazinylmethyl)-3-biphenylyl]methyl}octanamide tetrakis(trifluoroacetate);
N-{[3′-(1-piperazinylmethyl)-3-biphenylyl]methyl}-1,3-benzodioxole-5-carboxamide bis(trifluoroacetate);
methyl 3-{[({3′-[(1S,4S)-2,5-diazabicyclo[2.2.1]hept-2-ylmethyl]-3-biphenylyl}methyl)amino]carbonyl}benzoate bis(trifluoroacetate);
3-cyano-N-{[3′-[(1S,4S)-2,5-diazabicyclo[2.2.1]hept-2-ylmethyl]-6-(methyloxy)-3-biphenylyl]methyl}benzamide bis(trifluoroacetate);
N-({3′-[(1S,4S)-2,5-diazabicyclo[2.2.1]hept-2-ylmethyl]-3-biphenylyl}methyl)-3-(ethyloxy)benzamide bis(trifluoroacetate);
N-{[6-(methyloxy)-3′-(1-piperazinylmethyl)-3-biphenylyl]methyl}-1,3-benzodioxole-5-carboxamide bis(trifluoroacetate);
N-({3′-[(1S,4S)-2,5-diazabicyclo[2.2.1]hept-2-ylmethyl]-3-biphenylyl}methyl)-2,1,3-benzoxadiazole-5-carboxamide bis(trifluoroacetate);
N-({3′-[(3-methyl-1-piperazinyl)methyl]-3-biphenylyl}methyl)-3-(methylsulfonyl)benzamide bis(trifluoroacetate);
3-cyano-N-{[3′-(1-piperazinylmethyl)-3-biphenylyl]methyl}benzamide bis(trifluoroacetate);
N-{[3′-(1-piperazinylmethyl)-3-biphenylyl]methyl}-3-(trifluoromethyl)benzamide bis(trifluoroacetate);
3-(methyloxy)-N-{[3′-(1-piperazinylmethyl)-3-biphenylyl]methyl}benzamide bis(trifluoroacetate);
N-{[3′-[(1S,4S)-2,5-diazabicyclo[2.2.1]hept-2-ylmethyl]-6-(methyloxy)-3-biphenylyl]methyl}-1,3-benzodioxole-5-carboxamide bis(trifluoroacetate);
N-[(3′-{[(3R)-3-methyl-1-piperazinyl]methyl}-3-biphenylyl)methyl]-1,3-benzodioxole-5-carboxamide bis(trifluoroacetate);
3-cyano-N-[(3′-{[(3R)-3-methyl-1-piperazinyl]methyl}-3-biphenylyl)methyl]benzamide bis(trifluoroacetate);
N-({3′-[(1S,4S)-2,5-diazabicyclo[2.2.1]hept-2-ylmethyl]-3-biphenylyl}methyl)-1,3-benzodioxole-5-carboxamide bis(trifluoroacetate);
N-({3′-[(1S,4S)-2,5-diazabicyclo[2.2.1]hept-2-ylmethyl]-3-biphenylyl}methyl)-3-(methylsulfonyl)benzamide bis(trifluoroacetate);
3-chloro-N-{[3′-(1-piperazinylmethyl)-3-biphenylyl]methyl}benzamide bis(trifluoroacetate);
(E)-2-phenyl-N-{[3′-(1-piperazinylmethyl)-3-biphenylyl]methyl}ethenesulfonamide bis(trifluoroacetate);
3-cyano-N-({3′-[(1R,4R)-2,5-diazabicyclo[2.2.1]hept-2-ylmethyl]-3-biphenylyl}methyl)benzamide bis(trifluoroacetate);
N-({3′-[(1S,4S)-2,5-diazabicyclo[2.2.1]hept-2-ylmethyl]-3-biphenylyl}methyl)-3-(methyloxy)benzamide bis(trifluoroacetate);
N-{[3′-(hexahydro-1H-1,4-diazepin-1-ylmethyl)-3-biphenylyl]methyl}-1,3-benzodioxole-5-carboxamide bis(trifluoroacetate);
4-(methyloxy)-N-{[3′-(1-piperazinylmethyl)-3-biphenylyl]methyl}benzenesulfonamide bis(trifluoroacetate);
N-({3′-[(4-acetyl-1-piperazinyl)methyl]-3-biphenylyl}methyl)-1,3-benzodioxole-5-carboxamide trifluoroacetate;
N-({3′-[(2,5-dimethyl-1-piperazinyl)methyl]-3-biphenylyl}methyl)-1,3-benzodioxole-5-carboxamide bis(trifluoroacetate);
N-({3′-[(3-amino-1-pyrrolidinyl)methyl]-3-biphenylyl}methyl)-1,3-benzodioxole-5-carboxamide bis(trifluoroacetate);
3-cyano-N-({3′-[(2,5-dimethyl-1-piperazinyl)methyl]-3-biphenylyl}methyl)benzamide bis(trifluoroacetate);
N-({3′-[(3-pyrrolidinylamino)methyl]-3-biphenylyl}methyl)-1,3-benzodioxole-5-carboxamide bis(trifluoroacetate);
N-({3′-[(4-methyl-1-piperazinyl)methyl]-3-biphenylyl}methyl)-1,3-benzodioxole-5-carboxamide bis(trifluoroacetate);
N-[(6-fluoro-3′-{[(3S)-3-methyl-1-piperazinyl]methyl}-3-biphenylyl)methyl]-3-(phenylcarbonyl)benzamide;
3-(ethyloxy)-N-[(6-fluoro-3′-{[(3S)-3-methyl-1-piperazinyl]methyl}-3-biphenylyl)methyl]benzamide;
3-acetyl-N-[(4-fluoro-3′-{[(3S)-3-methyl-1-piperazinyl]methyl}-3-biphenylyl)methyl]benzamide;
3-(ethyloxy)-N-[(4-fluoro-3′-{[(3S)-3-methyl-1-piperazinyl]methyl}-3-biphenylyl)methyl]benzamide;
N-[(4-fluoro-3′-{[(3S)-3-methyl-1-piperazinyl]methyl}-3-biphenylyl)methyl]-3-(phenylcarbonyl)benzamide;
N-[(4-fluoro-3′-{[(3S)-3-methyl-1-piperazinyl]methyl}-3-biphenylyl)methyl]-1,3-benzodioxole-5-carboxamide;
3-(2-oxo-1-pyrrolidinyl)-N-{[3′-(1-piperazinylmethyl)-3-biphenylyl]methyl}benzamide;
methyl 2-[({[3′-(1-piperazinylmethyl)-3-biphenylyl]methyl}amino)carbonyl]benzoate;
3-[(4-chloro-1H-pyrazol-1-yl)methyl]-N-{[3′-(1-piperazinylmethyl)-3-biphenylyl]methyl}benzamide;
1-methyl-N-{[3′-(1-piperazinylmethyl)-3-biphenylyl]methyl}-1H-1,2,3-benzotriazole-6-carboxamide;
3-[(2-hydroxyethyl)oxy]-N-{[3′-(1-piperazinylmethyl)-3-biphenylyl]methyl}benzamide;
3-[acetyl(methyl)amino]-N-{[3′-(1-piperazinylmethyl)-3-biphenylyl]methyl}benzamide;
3-[(3,4-dichlorophenyl)carbonyl]-N-[(6-fluoro-3′-{[(3S)-3-methyl-1-piperazinyl]methyl}-3-biphenylyl)methyl]benzamide;
3-ethyl-N-[(6-fluoro-3′-{[(3S)-3-methyl-1-piperazinyl]methyl}-3-biphenylyl)methyl]benzamide;
N-[(6-fluoro-3′-{[(3S)-3-methyl-1-piperazinyl]methyl}-3-biphenylyl)methyl]benzamide trifluoroacetate;
3-[(2,5-dioxo-4-imidazolidinyl)methyl]-N-{[3′-(1-piperazinylmethyl)-3-biphenylyl]methyl}benzamide;
methyl {3-[({[3′-(1-piperazinylmethyl)-3-biphenylyl]methyl}amino)carbonyl]phenyl}acetate;
3-(3-amino-4,5-dihydro-1H-pyrazol-1-yl)-N-{[3′-(1-piperazinylmethyl)-3-biphenylyl]methyl}benzamide trifluoroacetate;
2′-methyl-N-{[3′-(1-piperazinylmethyl)-3-biphenylyl]methyl}-3-biphenylcarboxamide;
3-[(methylamino)sulfonyl]-N-{[3′-(1-piperazinylmethyl)-3-biphenylyl]methyl}benzamide;
N-methyl-N′-{[3′-(1-piperazinylmethyl)-3-biphenylyl]methyl}-1,3-benzenedicarboxamide;
3-(3,5-dimethyl-4-isoxazolyl)-N-{[3′-(1-piperazinylmethyl)-3-biphenylyl]methyl}benzamide;
3-[(methylsulfonyl)amino]-N-{[3′-(1-piperazinylmethyl)-3-biphenylyl]methyl}benzamide;
3-cyano-N-[(4-fluoro-3′-{[(3S)-3-methyl-1-piperazinyl]methyl}-3-biphenylyl)methyl]benzamide;
N-{[3′-(1-piperazinylmethyl)-3-biphenylyl]methyl}-2-(3-pyridinyl)-1,3-thiazole-4-carboxamide;
3-acetyl-N-{[3-(6-{[(3S)-3-methyl-1-piperazinyl]methyl}-2-pyridinyl)phenyl]methyl}benzamide trifluoroacetate;
N-{[3-(6-{[(3S)-3-methyl-1-piperazinyl]methyl}-2-pyridinyl)phenyl]methyl}-3-(phenylcarbonyl)benzamide trifluoroacetate;
3-acetyl-N-{[3-(5-{[(3S)-3-methyl-1-piperazinyl]methyl}-2-thienyl)phenyl]methyl}benzamide trifluoroacetate;
N-{[3-(6-{[(3S)-3-methyl-1-piperazinyl]methyl}-2-pyridinyl)phenyl]methyl}-1,3-benzodioxole-5-carboxamide trifluoroacetate;
3-(hydroxymethyl)-N-{[3′-(1-piperazinylmethyl)-3-biphenylyl]methyl}benzamide;
3-(ethyloxy)-N-{[3-(6-{[(3S)-3-methyl-1-piperazinyl]methyl}-2-pyridinyl)phenyl]methyl}benzamide trifluoroacetate;
N-{[3′-(1-piperazinylmethyl)-3-biphenylyl]methyl}-1,3-benzenedicarboxamide;
N-{[3′-(1-piperazinylmethyl)-3-biphenylyl]methyl}-8-quinolinecarboxamide;
3-(aminosulfonyl)-N-{[3′-(1-piperazinylmethyl)-3-biphenylyl]methyl}benzamide;
3-[(3,4-dichlorophenyl)carbonyl]-N-{[3-(6-{[(3S)-3-methyl-1-piperazinyl]methyl}-2-pyridinyl)phenyl]methyl}benzamide trifluoroacetate;
N-{[3′-(1-piperazinylmethyl)-3-biphenylyl]methyl}-6-(1H-pyrrol-1-yl)-3-pyridinecarboxamide; and
3-[(aminocarbonyl)amino]-N-{[3′-(1-piperazinylmethyl)-3-biphenylyl]methyl}benzamide;

or any other pharmaceutically acceptable salt, or non-salt form thereof.

The most preferred compounds are selected from the group consisting of:
N-[(6-fluoro-3′-{[(3S)-3-methyl-1-piperazinyl]methyl}-3-biphenylyl)methyl]-3-oxo-2,3-dihydro-1H-indene-5-carboxamide bis(trifluoroacetate);
N-[(6-fluoro-3′-{[(3S)-3-methyl-1-piperazinyl]methyl}-3-biphenylyl)methyl]-3-propanoylbenzamide bis(trifluoroacetate);
3-acetyl-N-[(6-fluoro-3′-{[(3S)-3-methyl-1-piperazinyl]methyl}-3-biphenylyl)methyl]benzamide bis(trifluoroacetate);
N-[(6-fluoro-3′-{[(3S)-3-methyl-1-piperazinyl]methyl}-3-biphenylyl)methyl]-3-(2-oxopropyl)benzamide bis(trifluoroacetate);
3-(ethyloxy)-N-({6-fluoro-3′-[(3-methyl-1-piperazinyl)methyl]-3-biphenylyl}methyl)benzamide bis(trifluoroacetate);
3-acetyl-N-[(3′-{[(3S)-3-methyl-1-piperazinyl]methyl}-3-biphenylyl)methyl]benzamide bis(trifluoroacetate);
3-cyano-N-[(6-fluoro-3′-{[(3S)-3-methyl-1-piperazinyl]methyl}-3-biphenylyl)methyl]benzamide bis(trifluoroacetate);
3-acetyl-N-[(6-(methyloxy)-3′-{[(3S)-3-methyl-1-piperazinyl]methyl}-3-biphenylyl)methyl]benzamide bis(trifluoroacetate);
3-cyano-N-[(3′-{[(3S)-3-methyl-1-piperazinyl]methyl}-3-biphenylyl)methyl]benzamide bis(trifluoroacetate);
N-({3′-[(1S,4S)-2,5-diazabicyclo[2.2.1]hept-2-ylmethyl]-6-fluoro-3-biphenylyl}methyl)-1,3-benzodioxole-5-carboxamide bis(trifluoroacetate);
3-(ethyloxy)-N-({6-(methyloxy)-3′-[(3-methyl-1-piperazinyl)methyl]-3-biphenylyl}methyl)benzamide bis(trifluoroacetate);
N-[(3′-{[(3S)-3-methyl-1-piperazinyl]methyl}-3-biphenylyl)methyl]-1,3-benzodioxole-5-carboxamide bis(trifluoroacetate);
3-acetyl-N-({3′-[(3-methyl-1-piperazinyl)methyl]-3-biphenylyl}methyl)benzamide bis(trifluoroacetate);
3-acetyl-N-[(3′-{[(3R)-3-methyl-1-piperazinyl]methyl}-3-biphenylyl)methyl]benzamide bis(trifluoroacetate);
N-{[6-fluoro-3′-(1-piperazinylmethyl)-3-biphenylyl]methyl}-1,3-benzodioxole-5-carboxamide bis(trifluoroacetate);
methyl 3-{[({3′-[(3-methyl-1-piperazinyl)methyl]-3-biphenylyl}methyl)amino]carbonyl}benzoate bis(trifluoroacetate);
3-cyano-N-({3′-[(1S,4S)-2,5-diazabicyclo[2.2.1]hept-2-ylmethyl]-6-fluoro-3-biphenylyl}methyl)benzamide bis(trifluoroacetate);
3-cyano-N-({3′-[(3-methyl-1-piperazinyl)methyl]-3-biphenylyl}methyl)benzamide bis(trifluoroacetate);
3-acetyl-N-{[3′-(1-piperazinylmethyl)-3-biphenylyl]methyl}benzamide bis(trifluoroacetate);
3-cyano-N-[(6-(methyloxy)-3′-{[(3S)-3-methyl-1-pipeprazinyl]methyl}-3-biphenylyl)methyl]benzamide bis(trifluoroacetate);
3-(ethyloxy)-N-{[3′-(1-piperazinylmethyl)-3-biphenylyl]methyl}benzamide bis(trifluoroacetate);
3-cyano-N-{[6-fluoro-4′-(methyloxy)-3′-(1-piperazinylmethyl)-3-biphenylyl]methyl}benzamide bis(trifluoroacetate);
N-({3′-[(3-methyl-1-piperazinyl)methyl]-3-biphenylyl}methyl)-1,3-benzodioxole-5-carboxamide bis(trifluoroacetate);
N-{[6-fluoro-4′-(methyloxy)-3′-(1-piperazinylmethyl)-3-biphenylyl]methyl}-1,3-benzodioxole-5-carboxamide bis(trifluoroacetate);
3-(ethyloxy)-N-({3′-[(3-methyl-1-piperazinyl)methyl]-3-biphenylyl}methyl)benzamide bis(trifluoroacetate);
3-acetyl-N-({3′-[(1S,4S)-2,5-diazabicyclo[2.2.1]hept-2-ylmethyl]-3-biphenylyl}methyl)benzamide bis(trifluoroacetate);
N-({3′-[(3-methyl-1-piperazinyl)methyl]-3-biphenylyl}methyl)-2,1,3-benzoxadiazole-5-carboxamide bis(trifluoroacetate);
N-{[3′-(1-piperazinylmethyl)-3-biphenylyl]methyl}-2,1,3-benzoxadiazole-5-carboxamide bis(trifluoroacetate);
N-{[4′,6-difluoro-3′-(1-piperazinylmethyl)-3-biphenylyl]methyl}-1,3-benzodioxole-5-carboxamide bis(trifluoroacetate),
3-(methyloxy)-N-({3′-[(3-methyl-1-piperazinyl)methyl]-3-biphenylyl}methyl)benzamide bis(trifluoroacetate);
3-cyano-N-{[4′,6-difluoro-3′-(1-piperazinylmethyl)-3-biphenylyl]methyl}benzamide bis(trifluoroacetate);
3-cyano-N-{[6-(methyloxy)-3′-(1-piperazinylmethyl)-3-biphenylyl]methyl}benzamide bis(trifluoroacetate);
methyl 3-[({[3′-(1-piperazinylmethyl)-3-biphenylyl]methyl}amino)carbonyl]benzoate bis(trifluoroacetate);
3-(methylsulfonyl)-N-{[3′-(1-piperazinylmethyl)-3-biphenylyl]methyl}benzamide bis(trifluoroacetate);
N-[3-(4-methyl-1-piperazinyl)propyl]-N-{[3′-(1-piperazinylmethyl)-3-biphenylyl]methyl}octanamide tetrakis(trifluoroacetate);
N-{[3′-(1-piperazinylmethyl)-3-biphenylyl]methyl}-1,3-benzodioxole-5-carboxamide bis(trifluoroacetate);
methyl 3-{[({3′-[(1S,4S)-2,5-diazabicyclo[2.2.1]hept-2-ylmethyl]-3-biphenylyl}methyl)amino]carbonyl}benzoate bis(trifluoroacetate);
3-cyano-N-{[3′-[(1S,4S)-2,5-diazabicyclo[2.2.1]hept-2-ylmethyl]-6-(methyloxy)-3-biphenylyl]methyl}benzamide bis(trifluoroacetate);
N-({3′-[(1S,4S)-2,5-diazabicyclo[2.2.1]hept-2-ylmethyl]-3-biphenylyl}methyl)-3-(ethyloxy)benzamide bis(trifluoroacetate);
N-[(6-fluoro-3′-{[(3S)-3-methyl-1-piperazinyl]methyl}-3-biphenylyl)methyl]-3-(phenylcarbonyl)benzamide;
3-(ethyloxy)-N-[(6-fluoro-3′-{[(3S)-3-methyl-1-piperazinyl]methyl}-3-biphenylyl)methyl]benzamide;
3-acetyl-N-[(4-fluoro-3′-{[(3S)-3-methyl-1-piperazinyl]methyl}-3-biphenylyl)methyl]benzamide;
3-(ethyloxy)-N-[(4-fluoro-3′-{[(3S)-3-methyl-1-piperazinyl]methyl}-3-biphenylyl)methyl]benzamide;
N-[(4-fluoro-3′-{[(3S)-3-methyl-1-piperazinyl]methyl}-3-biphenylyl)methyl]-3-(phenylcarbonyl)benzamide;
N-[(4-fluoro-3′-{[(3S)-3-methyl-1-piperazinyl]methyl}-3-biphenylyl)methyl]-1,3-benzodioxole-5-carboxamide;
3-(2-oxo-1-pyrrolidinyl)-N-{[3′-(1-piperazinylmethyl)-3-biphenylyl]methyl}benzamide;
methyl 2-[({[3′-(1-piperazinylmethyl)-3-biphenylyl]methyl}amino)carbonyl]benzoate;
3-[(4-chloro-1H-pyrazol-1-yl)methyl]-N-{[3′-(1-piperazinylmethyl)-3-biphenylyl]methyl}benzamide;
1-methyl-N-{[3′-(1-piperazinylmethyl)-3-biphenylyl]methyl}-1H-1,2,3-benzotriazole-6-carboxamide;
3-[(2-hydroxyethyl)oxy]-N-{[3′-(1-piperazinylmethyl)-3-biphenylyl]methyl}benzamide;
3-[acetyl(methyl)amino]-N-{[3′-(1-piperazinylmethyl)-3-biphenylyl]methyl}benzamide;
3-[(3,4-dichlorophenyl)carbonyl]-N-[(6-fluoro-3′-{[(3S)-3-methyl-1-piperazinyl]methyl}-3-biphenylyl)methyl]benzamide;
3-ethyl-N-[(6-fluoro-3′-{[(3S)-3-methyl-1-piperazinyl]methyl}-3-biphenylyl)methyl]benzamide;
N-[(6-fluoro-3′-{[(3S)-3-methyl-1-piperazinyl]methyl}-3-biphenylyl)methyl]benzamide trifluoroacetate; and
3-[(2,5-dioxo-4-imidazolidinyl)methyl]-N-{[3′-(1-piperazinylmethyl)-3-biphenylyl]methyl}benzamide;

or any other pharmaceutically acceptable salt, or non-salt form thereof.

### Methods of Preparation

### Preparation

The compounds of Formula (I) may be obtained by applying synthetic procedures, some of which are illustrated in the Schemes below. The synthesis provided for these Schemes is applicable for producing compounds of Formula (I) having a variety of different R1, R2, and R3, which are reacted, employing substituents which are suitable protected, to achieve compatibility with the reactions outlined herein. Subsequent deprotection, in those cases, then affords compounds of the nature generally disclosed. While some Schemes are shown with specific compounds, this is merely for illustration purpose only.

### Preparation 1

As shown in Scheme 1, bromo benzylamines 1 were loaded onto 2,6-dimethoxy-4-polystyrenebenzyloxy-benzaldehyde (DMHB resin) via reductive amination. The resin-bound amines 2 were reacted with various sulfonyl chlorides to yield sulfonamides 3, which underwent Suzuki coupling with substituted formyl phenyl boronic acids to give biphenylaldehydes 4. Reductive alkylation of 4 with amines, followed by cleavage with 20% of trifluoroacetic acid in dichoroethane, afforded desired products 5.

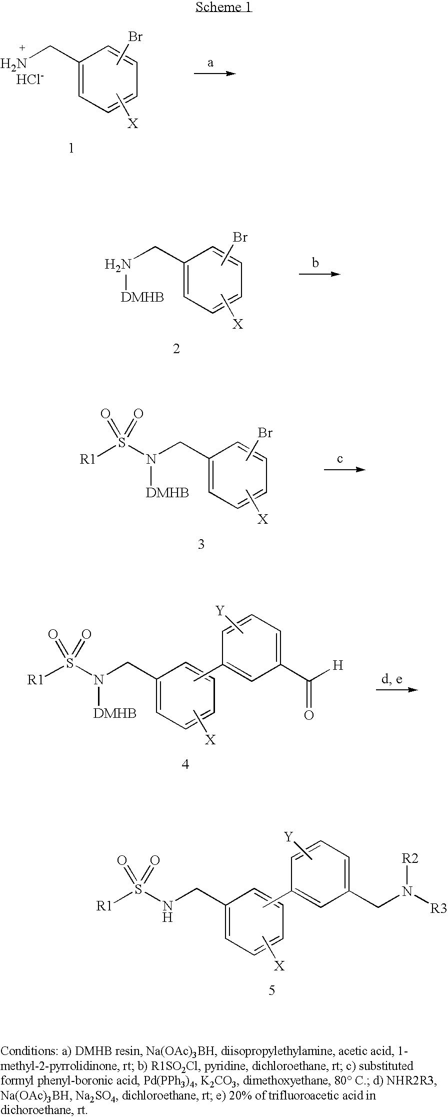

### SYNTHETIC EXAMPLES

The invention will now be described by reference to the following Examples which are merely illustrative and are not to be construed as a limitation of the scope of the present invention. Most reagents and intermediates are commercially available or are prepared according to procedures in the literature. The preparation of intermediates not described in the literature is illustrated below.

Flash column chromatography was carried out using Merck 9385 silica unless stated otherwise.

LC/MS analyses were conducted under the following conditions unless stated otherwise.

| Column: | 3.3 cm × 4.6 mm ID, 3um ABZ + PLUS | | |
|---|---|---|---|
| Flow Rate: | 3 ml/min | | |
| Injection | 5 μl | | |
| Volume: | | | |
| Temp: | Room temperature | | |
| Solvents: | A: 0.1% Formic Acid + 10 mMolar | | |
| | Ammonium Acetate. | | |
| | B: 95% Acetonitrile + 0.05% Formic Acid | | |
| | Time | A % | B % |
| Gradient: | 0.00 | 100 | 0 |
| | 0.70 | 100 | 0 |
| | 4.20 | 0 | 100 |
| | 5.30 | 0 | 100 |
| | 5.50 | 100 | 0 |

The Mass Directed Automated Preparative (MDAP) was conducted under the conditions described in System A or in System B unless stated otherwise.

System A: Formate salts
The preparative column used was a Supelcosil ABZplus (10 cm×2.12 cm internal diameter; particle size 5 m)
UV detection wavelength: 200-320 nM
Flow rate: 20 ml/min
Injection Volume: 0.5 ml
Solvent A: 0.1% formic acid
Solvent B: 95% acetonitrile+0.05% formic acid

### System B TFA Salts

The preparative column used was a Supelcosil ABZplus (10 cm×2.12 cm internal diameter; particle size 5 m)
UV detection wavelength: 200-320 nM
Flow rate: 20 ml/min
Injection Volume: 0.5 ml
Solvent A: water+0.1% trifluoroacetic acid
Solvent B: acetonitrile+0.1% trifluoroacetic acid

The Gilson preparatory HPLC was conducted under the following conditions unless stated otherwise.
Column: 75×33 mm 1. D., S-5 um, 12 nm
Flow rate: 30 mL/min
Injection Volume: 0.800 mL
Room temperature
Solvent A: 0.1% trifluoroacetic acid in water
Solvent B: 0.1% trifluoroacetic acid in acetonitrile

### Example 1

### Preparation of 4-(methyloxy)-N-{[3′-(1-piperazinylmethyl)-3-biphenyl]methyl}benzenesulfonamide

### a) DMHB resin-bound 3-bromo-benzylamine

To a 250 mL shaker vessel was added 2,6-dimethoxy-4-polystyrenebenzyloxy-benzaldehyde (DMHB resin) (10 g, 1.5 mmol/g, 15 mmol) and 150 mL of 1-methyl-2-pyrrolidinone (NMP). 3-Bromo-benzylamine HCl salt (17 g, 75 mmol), diisopropylethylamine (DIEA) (13 mL, 75 mmol), acetic acid (HOAc) (15 mL), and Na(OAc)₃BH (19.1 g, 90 mmol) were then added. The resulting mixture was shaken at rt for overnight, and was then washed with NMP (150 mL×2), dichloromethane (DCM) (150 mL×2), MeOH (150 mL×2) and DCM (150 mL×2). The resulting resin was dried in vacuum oven at 35° C. for overnight to yield DMHB resin-bound 3-bromo-benzylamine (15 mmol).

### b) 4-(Methyloxy)-N-{[3′-(1-piperazinylmethyl)-3-biphenylyl]methyl}benzenesulfonamide

To a mixture of the above resin-bound 3-bromo-benzylamine (1a, 2 g, 1.2 mmol/g (theoretical loading), 2.4 mmol) in 80 mL of dichloroethane (DCE) was added 4-methoxybenzenesulfonyl chloride (5.0 g, 24 mmol) and pyridine (13 mL, 160 mmol). The mixture was shaken at rt for overnight, and was then washed with DCM (100 mL×2), MeOH (100 mL×2) and DCM (100 mL×2). The resulting resin was dried in vacuum oven at 35° C. for overnight. An analytical amount of the resin was cleaved with 20% of trifluoroacetic acid in DCE for 10 min. The resulting solution was concentrated in vacuo and dissolved in 0.5 mL of MeOH. MS (ESI): 356 [M+H]⁺.

To a mixture of the above resin-bound N-[(3-bromophenyl)methyl]-4-(methyloxy)benzenesulfonamide (3.38 g, 0.99 mmol/g (theoretical loading), 3.35 mmol) in 83 mL of dimethoxyethane (DME) was added 3-formylphenyl boronic acid (1.49 g, 9.93 mmol), 2 M K₂CO₃ aqueous solution (5 mL, 9.93 mmol) and Pd(PPh₃)₄ (0.19 g, 0.17 mmol). After purged with argon for 5-10 min, the mixture was heated at 80° C. for 10 h under argon. The resin was then washed with tetrahydrofuran (THF) (100 mL×2), THF:H₂O (1:1, 100 mL×2), H₂O (100 mL×2), THF:H₂O (1:1, 100 mL×2), THF (100 mL×2), DCM (100 mL×2), and dried in vacuum oven at 35° C. for overnight. An analytical amount of the resin was cleaved with 20% of TFA in DCE for 10 min. The resulting solution was concentrated in vacuo and dissolved in 0.5 mL of CH₃CN. MS (ESI): 382 [M+H]⁺.

To a mixture of the above resin-bound N-[(3′-formyl-3-biphenylyl)methyl]-4-(methyloxy)benzenesulfonamide (400 mg, 0.97 mmol/g (theoretical loading), 0.388 mmol) in 17 mL of DCE was added Na₂SO₄ (0.24 g, 1.68 mmol) and 1,1-dimethylethyl 1-piperazinecarboxylate (0.31 g, 1.68 mmol). After shaking at rt for 10 min, Na(OAc)₃BH (0.43 g, 2.02 mmol) was added. The mixture was shaken at rt for overnight, and was then washed with THF (100 mL×2), THF:H₂O (1:1, 100 mL×2), H₂O (100 mL×2), THF:H₂O (1:1, 100 mL×2), THF (100 mL×2), DCM (100 mL×2). The resulting resin was dried in vacuum oven at 35° C. for overnight and was cleaved with 6 mL of 20% of TFA in DCE for 30 min and treated again with 6 mL of 20% of TFA in DCE for 30 min. The combined cleavage solution was concentrated in vacuo. The residue was dissolved in DMSO and purified using a Gilson semi-preparative HPLC system with a YMC ODS-A (C-18) column 50 mm by 20 mm ID, eluting with 10% B to 90% B in 3.2 min, hold for 1 min where A=H₂O (0.1% trifluoroacetic acid) and B=CH₃CN (0.1% trifluoroacetic acid) pumped at 25 mL/min, to produce 4-(methyloxy)-N-{[3′-(1-piperazinylmethyl)-3-biphenylyl]methyl}benzenesulfonamide as a bis-trifluoroacetate salt (white powder, 80 mg, 46% over 5 steps). MS (ESI): 452 [M+H]⁺.

Proceeding in a similar manner, but replacing 4-methoxybenzene sulfonyl chloride with the appropriate sulfonyl chlorides, and/or replacing 1,1-dimethylethyl 1-piperazinecarboxylate with the appropriate amines, the compounds listed in Tables 1 and 2 were prepared.

TABLE 1

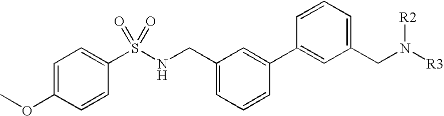

| Example | NR2R3 | MS [M + H]⁺ |
|---|---|---|
| 2 | pyrrolidin-1-yl | 437 |
| 3 | hexahydro-azepin-1-yl | 465 |
| 4 | 4-methyl-piperazin-1-yl | 466 |
| 5 | 4-methyl-hexahydro-1,4-diazepin-1-yl | 480 |
| 6 | 4-ethyl-piperazin-1-yl | 480 |
| 7 | 3-amino-pyrrolidin-1-yl | 452 |
| 8 | N,N-diethyl-amino | 439 |
| 9 | N-[2-(dimethylamino)ethyl],N-methyl-amino | 468 |
| 10 | hexahydro-1,4-diazepin-1-yl | 466 |
| 11 | (1S,4S)-2,5-diazabicyclo[2.2.1]hept-2-yl | 464 |
| 12 | N-piperidin-3-yl-amino | 466 |
| 13 | N-pyrrolidin-3-yl-amino | 452 |
| 14 | piperidin-1-yl | 451 |

TABLE 2

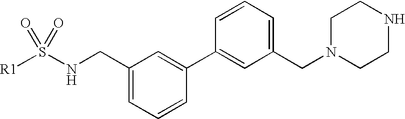

| Example | R1 | MS [M + H]⁺ |
|---|---|---|
| 15 | 2,3-dihydro-1,4-benzodioxin-6-yl | 480 |
| 16 | 3,4-methylenedioxy-phenyl | 466 |
| 17 | (1E)-2-phenyl-ethen-1-yl | 448 |
| 18 | 4-(1-methylethyl)-phenyl | 464 |
| 19 | 4-n-propyl-phenyl | 464 |
| 20 | 3-chloro-phenyl | 456 |
| 21 | 2-naphthyl | 472 |
| 22 | 5-(N,N-dimethylamino)-1-naphthyl | 515 |
| 23 | 3,4-dimethoxy-phenyl | 482 |
| 24 | 3-trifluoromethyl-phenyl | 490 |
| 25 | 4-trifluoromethyl-phenyl | 490 |
| 26 | 4-(trifluoromethoxy)-phenyl | 506 |
| 27 | 4-biphenyl | 498 |
| 28 | 8-quinolinyl | 473 |
| 29 | 1-naphthyl | 472 |
| 30 | 2-fluoro-phenyl | 440 |
| 31 | 4-chloro-2,5-dimethyl-phenyl | 484 |
| 32 | 2,4,6-trimethyl-phenyl | 464 |
| 33 | 2-trifluoromethyl-phenyl | 490 |
| 34 | 2,5-dimethoxy-phenyl | 482 |
| 35 | 4-fluoro-phenyl | 440 |

### Preparation 2

The resin-bound bromobenzylamines 2 were reacted with acids to yield amides 6, which underwent Suzuki coupling with substituted formyl phenyl boronic acids to give biphenylaldehydes 7 (Scheme 2). Reductive alkylation of 7 with amines, followed by cleavage, afforded desired products 8.

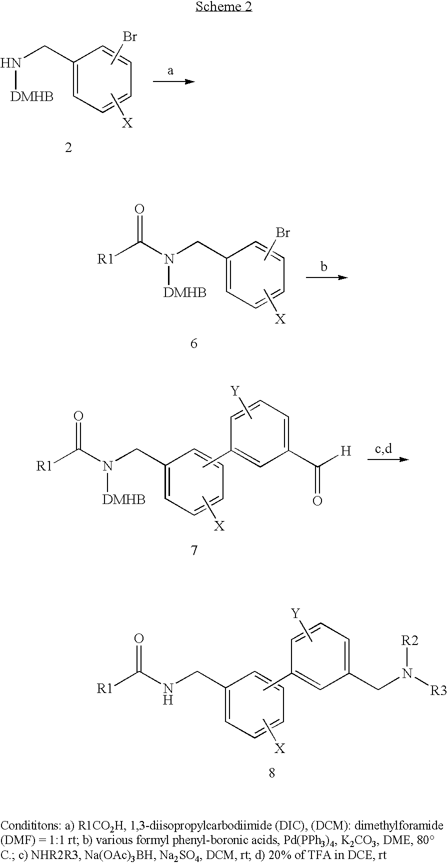

### Example 36

### Preparation of N-{[3′-(1-piperazinylmethyl)-3-biphenylyl]methyl}-1,3-benzodioxole-5-carboxamide

### a) DMHB resin-bound N-[(3-bromophenyl)methyl]-1,3-benzodioxole-5-carboxamide

To a mixture of DMHB resin-bound 3-bromo-benzylamine (1a, 2 g, 1.2 mmol/g (theoretical loading), 2.4 mmol) in DCE/DMF (1:1, 80 mL) was added piperonylic acid (4.0 g, 24 mmol) and DIC (3.7 mL, 24 mmol). The mixture was shaken at rt for overnight and was then washed with DMF (100 mL×2), DCM (100 mL×2), MeOH (100 mL×2) and DCM (100 mL×2). The resulting resin was dried in vacuum oven at 35° C. for overnight to yield DMHB resin-bound N-[(3-bromophenyl)methyl]-1,3-benzodioxole-5-carboxamide (2.4 mmol). An analytical amount of the resin was cleaved with 20% of TFA in DCE for 10 min. The resulting solution was concentrated in vacuo and dissolved in 0.5 mL of MeOH. MS (ESI): 334 [M+H]⁺.

### b) N-{[3′-(1-piperazinylmethyl)-3-biphenylyl]methyl}-1,3-benzodioxole-5-carboxamide

To a mixture of DMHB resin-bound N-[(3-bromophenyl)methyl]-1,3-benzodioxole-5-carboxamide (36a, 3.03 g, 1.0 mmol/g (theoretical loading), 3.03 mmol) in 76 mL of DME was added 3-formylphenyl boronic acid (1.36 g, 9.09 mmol), 2 M K₂CO₃ aqueous solution (4.5 mL, 9.09 mmol), and Pd(PPh₃)₄ (0.18 g, 0.15 mmol). After purged with argon for 5-10 min, the mixture was heated at 80° C. under argon for 10 h. The resulting resin was washed with THF (100 mL×2), THF:H₂O (1:1, 100 mL×2), H₂O (100 mL×2), THF:H₂O (1:1, 100 mL×2), THF (100 mL×2), DCM (100 mL×2), and dried in vacuum oven at 35° C. for overnight. An analytical amount of the resin was cleaved with 20% of TFA in DCM for 10 min. The resulting solution was concentrated in vacuo and dissolved in 0.5 mL of CH₃CN. MS (ESI): 360 [M+H]⁺.

To a mixture of the above resin (400 mg, 0.99 mmol/g, 0.40 mmol) in 17 mL of DCE was added Na₂SO₄ (0.24 g, 1.7 mmol) and 1,1-dimethylethyl 1-piperazinecarboxylate (0.32 g, 1.7 mmol). After shaking for 10 min, Na(OAc)₃BH (0.43 g, 2.04 mmol) was added. After shaken at rt for overnight, the resin was washed with THF (100 mL×2), THF:H₂O (1:1, 100 mL×2), H₂O (100 mL×2), THF:H₂O (1:1, 100 mL×2), THF (100 mL×2), DCM (100 mL×2) and dried in vacuum oven at 35° C. for overnight. The resulting resin was cleaved with 8 mL of 20% of TFA in DCE for 30 min and treated again with 8 mL of 20% of TFA in DCE for 30 min. The combined cleavage solution was concentrated in vacuo. The residue was dissolved in DMSO and purified using a Gilson semi-preparative HPLC system with a YMC ODS-A (C-18) column 50 mm by 20 mm ID, eluting with 10% B to 90% B in 3.2 min, hold for 1 min where A=H₂O (0.1% trifluoroacetic acid) and B=CH₃CN (0.1% trifluoroacetic acid) pumped at 25 mL/min, to produce N-{[3′-(1-piperazinylmethyl)-3-biphenylyl]methyl}-1,3-benzodioxole-5-carboxamide as a bis-trifluoroacetate salt (white powder, 100 mg, 58% over 5 steps). MS (ESI): 430 [M+H]⁺;

Proceeding in a similar manner, but replacing piperonylic acid with the appropriate acids, and/or replacing 1,1-dimethylethyl 1-piperazinecarboxylate with the appropriate amines, and/or replacing 3-bromo-benzylamine with appropriate bromobenzylamines, and/or replacing 3-formylphenyl-boronic acid with appropriate formylphenyl boronic acids, the compounds listed in Tables 3-13 were prepared.

TABLE 3

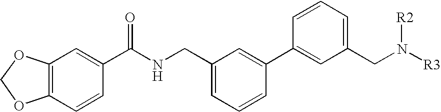

| | | MS |
|---|---|---|
| Example | NR2R3 | [M + H]⁺ |
| 37 | pyrrolidin-1-yl | 415 |
| 38 | hexahydro-azepin-1-yl | 443 |
| 39 | 4-methyl-piperazin-1-yl | 444 |
| 40 | 4-methyl-hexahydro-1,4-diazepin-1-yl | 458 |
| 41 | 4-acetyl-piperazin-1-yl | 472 |
| 42 | 4-ethyl-piperazin-1-yl | 458 |
| 43 | 3-amino-pyrrolidin-1-yl | 430 |
| 44 | N,N-diethy-lamino | 417 |
| 45 | 4-methyl-piperidin-1-yl | 443 |
| 46 | hexahydro-1,4-diazepin-1-yl | 444 |
| 47 | 3-amino-piperidin-1-yl | 444 |
| 48 | (1S,4S)-2,5-diazabicyclo[2.2.1]hept-2-yl | 442 |
| 49 | N-methyl,N-butyl-amino- | 431 |
| 50 | N-piperidin-3-yl-amino | 444 |
| 51 | N-piperidin-4-yl-amino | 444 |
| 52 | N-(hexahydro-azepin-4-yl)-amino | 458 |
| 53 | N-pyrrolidin-3-yl-amino | 430 |
| 54 | N-[3-(dimethylamino)propyl],N-methyl-amino | 460 |
| 55 | piperidin-1-yl | 429 |
| 56 | 4-formylpiperazin-1-yl | 458 |

TABLE 4

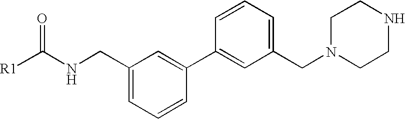

| Example | R1 | MS [M + H]⁺ |
|---|---|---|
| 57 | 3,4-dimethoxy-phenyl | 446 |
| 58 | 3,4-dichloro-phenyl | 454 |
| 59 | 2-naphthyl | 436 |
| 60 | 2-quinolinyl | 437 |
| 61 | 3-quinolinyl | 437 |
| 62 | 4-quinolinyl | 437 |
| 63 | 4-pyridinyl | 387 |
| 64 | 3-pyridinyl | 387 |
| 65 | 2-pyridinyl | 387 |
| 66 | benzofuran-2-yl | 426 |
| 67 | benzothiophen-2-yl | 442 |
| 68 | 1H-indol-2-yl | 425 |
| 69 | 1H-indol-3-yl | 425 |
| 70 | 2-furanyl | 376 |
| 71 | thiophen-3-yl | 392 |
| 72 | 3-furanyl | 376 |
| 73 | 2-methoxy-phenyl | 416 |
| 74 | 2-cyano-phenyl | 411 |
| 75 | 2-trifluoromethyl-phenyl | 454 |
| 76 | 2-chloro-phenyl | 420 |
| 77 | 3-methoxy-phenyl | 416 |
| 78 | 3-cyano-phenyl | 411 |
| 79 | 3-trifluoromethyl-phenyl | 454 |
| 80 | 3-chloro-phenyl | 420 |
| 81 | 4-methoxy-phenyl | 416 |
| 82 | 4-cyano-phenyl | 411 |
| 83 | 4-trifluoromethyl-phenyl | 454 |
| 84 | 4-chloro-phenyl | 420 |
| 85 | cyclopropyl | 350 |
| 86 | cyclohexyl | 392 |
| 87 | 1-naphthyl | 436 |
| 88 | 2,3-methylenedioxy-phenyl | 430 |
| 89 | 4-biphenyl | 462 |
| 90 | 4-(1,1-dimethylethyl)-phenyl | 442 |
| 91 | 4-(but-1-yl)-phenyl | 442 |
| 92 | 1-propyl | 352 |
| 93 | 1-butyl | 366 |
| 94 | 1-pentyl | 380 |
| 95 | 1-hexyl | 394 |
| 96 | 1-heptyl | 408 |
| 97 | thiophen-2-yl | 392 |
| 98 | 3-acetyl-phenyl | 428 |
| 99 | 3-ethoxy-phenyl | 430 |
| 100 | 3-(methoxycarbonyl)-phenyl | 444 |
| 101 | 3-methylsulfonyl-phenyl | 464 |
| 102 | 2,2-difluoro-benzo[1,3]dioxol-5-yl | 466 |
| 103 | benzothiazol-6-yl | 443 |
| 104 | 3,4-dihydro-2H-benzo[b][1,4]dioxepin-7-yl | 458 |
| 105 | benzo[1,2,5]oxadiazol-5-yl | 428 |
| 106 | 2,3-dihydro-benzo[1,4]dioxin-6-yl | 444 |
| 107 | 2,3-dihydro-benzofuran-5-yl | 428 |
| 108 | 3-(phenylcarbonyl)-phenyl | 490 |
| 109 | 3-methyl-phenyl | 400 |
| 110 | 3-acetylamino-phenyl | 443 |
| 111 | 4-hydroxy-3-methoxy-phenyl | 432 |
| 112 | 3-ethyl-phenyl | 414 |
| 113 | 3-trifluoromethoxy-phenyl | 470 |
| 114 | 3-fluoro-phenyl | 404 |
| 115 | 3-(toluene-4-sulfonylamino)-phenyl | 555 |
| 116 | 3-hydroxy-phenyl | 402 |
| 117 | 3-phenoxy-phenyl | 478 |
| 118 | 3H-benzoimidazol-5-yl | 426 |
| 119 | 4-methoxy-3-hydroxy-phenyl | 432 |
| 120 | phenyl | 386 |
| 121 | phenoxymethyl | 416 |
| 122 | 2-methyl-propen-1-yl | 364 |
| 123 | 2-phenyl-ethen-1-yl | 412 |
| 124 | 4-nitro-phenyl | 431 |

TABLE 5

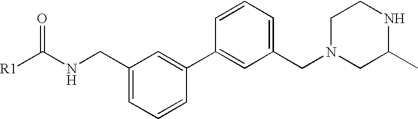

| Example | R1 | MS [M + H]⁺ |
|---|---|---|
| 125 | 3-acetyl-phenyl | 442 |
| 126 | 3-ethoxy-phenyl | 444 |
| 127 | 3-(methoxycarbonyl)-phenyl | 458 |
| 128 | 3-methylesulfonyl-phenyl | 478 |
| 129 | 2,2-difluoro-benzo[1,3]dioxol-5-yl | 480 |
| 130 | benzothiazol-6-yl | 457 |
| 131 | 3,4-dihydro-2H-benzo[b][1,4]dioxepin-7-yl | 472 |
| 132 | benzo[1,2,5]oxadiazol-5-yl | 442 |
| 133 | 2,3-dihydro-benzo[1,4]dioxin-6-yl | 458 |
| 134 | 2,3-dihydro-benzofuran-5-yl | 442 |
| 135 | 3-(phenylcarbonyl)-phenyl | 504 |
| 136 | 3-methyl-phenyl | 414 |
| 137 | 3-acetylamino-phenyl | 457 |
| 138 | 4-hydroxy-3-methoxy-phenyl | 446 |
| 139 | 3-ethyl-phenyl | 428 |
| 140 | 3-trifluoromethoxy-phenyl | 484 |
| 141 | 3-fluoro-phenyl | 418 |
| 142 | 3-(toluene-4-sulfonylamino)-phenyl | 569 |
| 143 | 3-hydroxy-phenyl | 416 |
| 144 | 3-phenoxy-phenyl | 492 |
| 145 | 3H-benzoimidazol-5-yl | 440 |
| 146 | 4-methoxy-3-hydroxy-phenyl | 446 |

TABLE 6

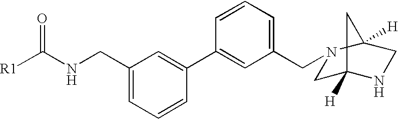

| Example | R1 | MS [M + H]⁺ |
|---|---|---|
| 147 | 3-acetyl-phenyl | 440 |
| 148 | 3-ethoxy-phenyl | 442 |
| 149 | 3-(methoxycarbony)-phenyl | 456 |
| 150 | 3-methylsulfonyl-phenyl | 476 |
| 151 | 2,2-difluoro-benzo[1,3]dioxol-5-yl | 478 |
| 152 | benzothiazol-6-yl | 455 |
| 153 | 3,4-dihydro-2H-benzo[b][1,4]dioxepin-7-yl | 470 |
| 154 | benzo[1,2,5]oxadiazol-5-yl | 440 |
| 155 | 2,3-dihydro-benzo[1,4]dioxin-6-yl | 456 |
| 156 | 2,3-dihydro-benzofuran-5-yl | 440 |
| 157 | 3-(phenylcarbonyl)-phenyl | 502 |
| 158 | 3-methyl-phenyl | 412 |
| 159 | 3-acetylamino-phenyl | 455 |
| 160 | 4-hydroxy-3-methoxy-phenyl | 444 |
| 161 | 3-ethyl-phenyl | 426 |
| 162 | 3-trifluoromethoxy-phenyl | 482 |
| 163 | 3-fluoro-phenyl | 416 |
| 164 | 3-(toluene-4-sulfonylamino)-phenyl | 567 |
| 165 | 3-hydroxy-phenyl | 414 |
| 166 | 3-phenoxy-phenyl | 490 |
| 167 | 3H-benzoimidazol-5-yl | 438 |
| 168 | 4-methoxy-3-hydroxy-phenyl | 444 |

TABLE 7

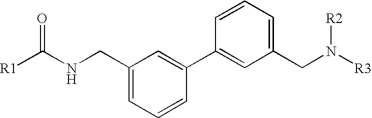

| Example | R1 | NR2R3 | MS [M + H]⁺ |
|---|---|---|---|
| 169 | 3-cyano-phenyl | (1S,4S)-2,5- | 423 |
| | | diazabicyclo[2.2.1]hept-2-yl | |
| 170 | 3-methoxy-phenyl | (1S,4S)-2,5- | 428 |
| | | diazabicyclo[2.2.1]hept-2-yl | |
| 171 | 3,4-methylenedioxy-phenyl | 1-azabicyclo[2.2.2]oct-3-yl | 470 |
| 172 | 3-cyano-phenyl | 1-azabicyclo[2.2.2]oct-3-yl | 451 |
| 173 | 3-cyano-phenyl | hexahydro-1,4-diazepin-1-yl | 425 |
| 174 | 3-methoxy-phenyl | hexahydro-1,4-diazepin-1-yl | 430 |
| 175 | 3,4-methylenedioxy-phenyl | 2,5-dimethyl-piperazin-1-yl | 458 |
| 176 | 3-cyano-phenyl | 2,5-dimethyl-piperazin-1-yl | 439 |
| 177 | 3-methoxy-phenyl | 2,5-dimethyl-piperazin-1-yl | 444 |
| 178 | 3,4-methylenedioxy-phenyl | 3-methyl-piperazin-1-yl | 444 |
| 179 | 3-cyano-phenyl | 3-methyl-piperazin-1-yl | 425 |
| 180 | 3-methoxy-phenyl | 3-methyl-piperazin-1-yl | 430 |
| 181 | 3,4-methylenedioxy-phenyl | 3,5-dimethyl-piperazin-1-yl | 458 |
| 182 | 3-cyano-phenyl | 3,5-dimethyl-piperazin-1-yl | 439 |
| 183 | 3-methoxy-phenyl | 3,5-dimethyl-piperazin-1-yl | 444 |

TABLE 8

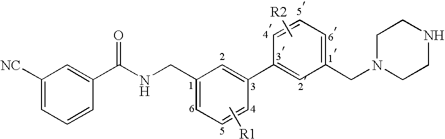

| Example | R1 | R2 | MS [M + H]⁺ |
|---|---|---|---|
| 184 | hydrogen | 4′-methoxy | 441 |
| 185 | hydrogen | 6′-fluoro | 429 |
| 186 | hydrogen | 6′-methoxy | 441 |

TABLE 9

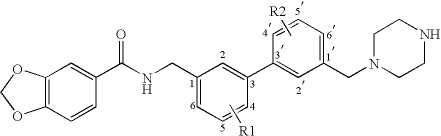

| Example | R1 | R2 | MS [M + H]⁺ |
|---|---|---|---|
| 187 | hydrogen | 4′-methoxy | 460 |
| 188 | hydrogen | 6′-fluoro | 448 |
| 189 | hydrogen | 6′-methoxy | 460 |

TABLE 10

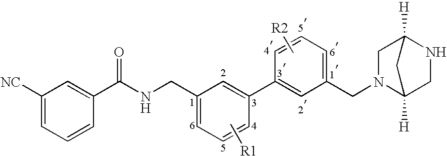

| Example | R1 | R2 | MS [M + H]⁺ |
|---|---|---|---|
| 190 | hydrogen | 4′-methoxy | 453 |
| 191 | hydrogen | 6′-fiuoro | 441 |
| 192 | hydrogen | 6′-methoxy | 453 |

TABLE 11

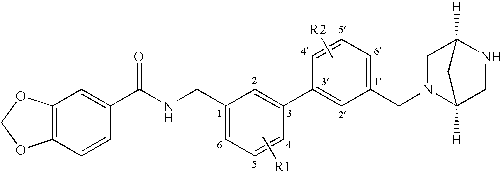

| | | | MS |
|---|---|---|---|
| Example | R1 | R2 | [M + H]⁺ |
| 193 | hydrogen | 4′-methoxy | 472 |
| 194 | hydrogen | 6′-fluoro | 460 |
| 195 | hydrogen | 6′-methoxy | 472 |

TABLE 12

| | | MS |
|---|---|---|
| Example | Compound | [M + H]⁺ |
| 196 | 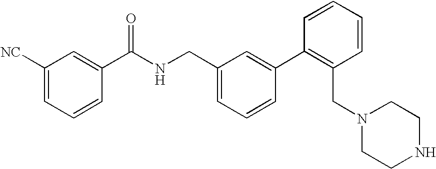 | 411 |
| 197 | 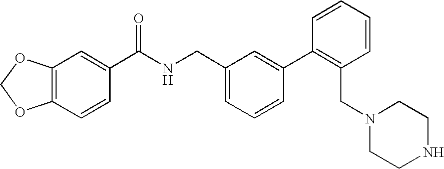 | 430 |
| 198 | 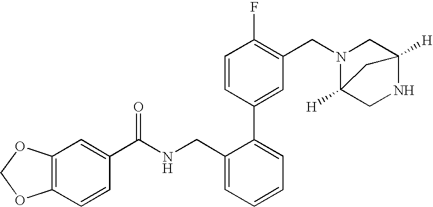 | 460 |
| 199 | 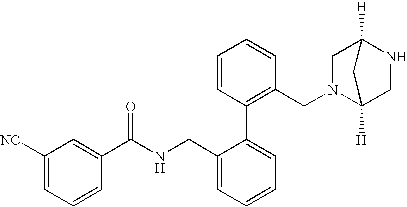 | 423 |
| 200 | 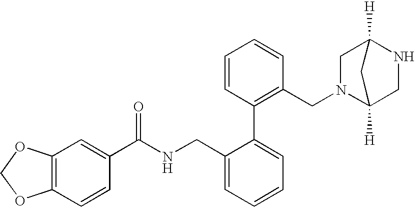 | 442 |

TABLE 13

| | | MS |
|---|---|---|
| Example | Compound | [M + H]⁺ |
| 201 | 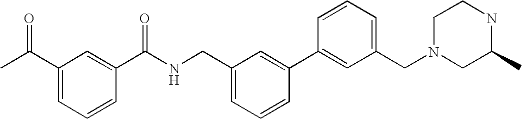 | 442 |
| 202 | 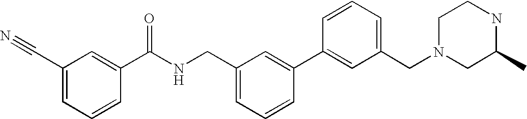 | 425 |
| 203 | 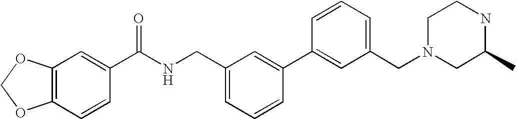 | 444 |
| 204 | 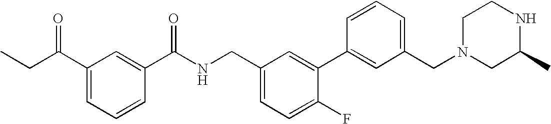 | 474 |
| 205 | 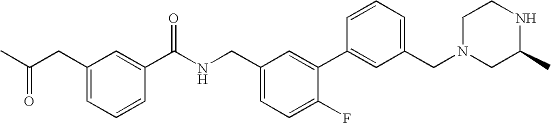 | 474 |
| 206 | 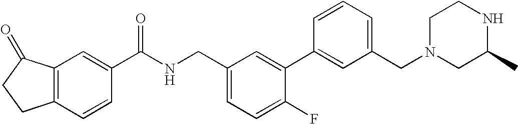 | 472 |
| 207 | 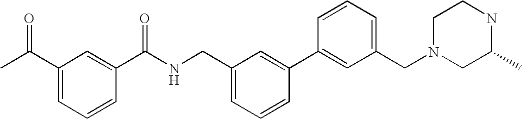 | 442 |
| 208 | 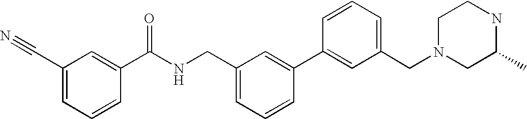 | 425 |
| 209 | 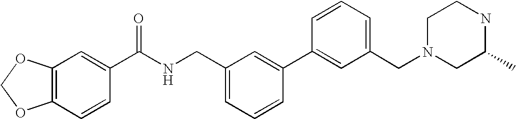 | 444 |
| 210 | 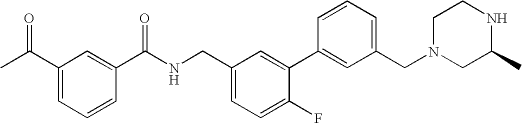 | 460 |
| 211 | 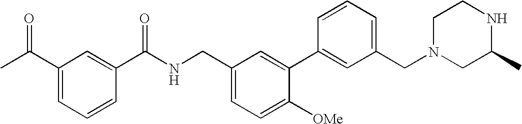 | 472 |
| 212 | 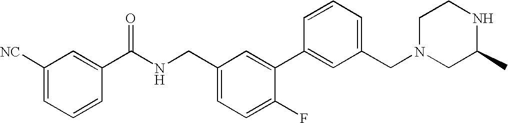 | 443 |
| 213 | 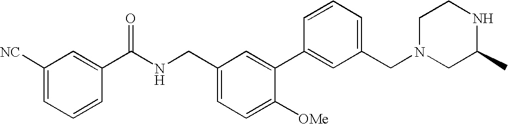 | 455 |
| 214 | 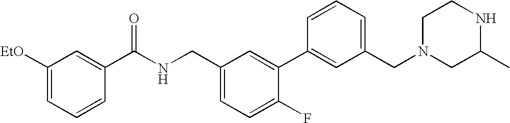 | 462 |
| 215 | 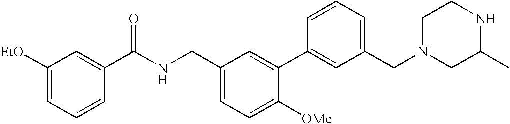 | 474 |

6-Carboxy-1-indanone used as the starting material of example 206 was prepared according to the following procedure: 3-(4-carboxyphenyl)propionic acid (5 g, 0.026 mol), frash AlCl₃ (25 g, 7.2 eq, 0.187 mol), and NaCl (2.5 g, 10% w/w of AlCl₃ used) were loaded into a 100 mL flask fitted with a condenser and internal thermometer going to the bottom of the flask. The flask was shaken briefly to mix the solids, then heated in an oil bath set to 190° C. Internal temperature was held at or above 180° C. for 1 h (reaction will fuse to form a dark brown liquid), then the mixture was cooled, and washed with water into a 2000 mL beaker containing ice. 180 mL of 6 M HCl and 250 mL of EtOAc were added. The layers were separated and the aqueous layer extracted with EtOAc (3×200 mL). Combined organic layers were washed with 2 M HCl, water, and brine, dried with MgSO₄, filtered and concentrated in vacuo to yield 6-carboxy-1-indanone (4.10 g, 90%) as a light brown solid directly used for the next step synthesis.

### Preparation 3

Chloro substituted benzylamines 9 were loaded onto the DMHB resin (Scheme 3). The resin-bound amines 10 were reacted with acids to yield amides 11, which underwent Suzuki coupling (using different conditions from preparation 2) to give biphenylaldehydes 7. Reductive alkylation of 7 with amines, followed by cleavage, afforded the desired products 8.

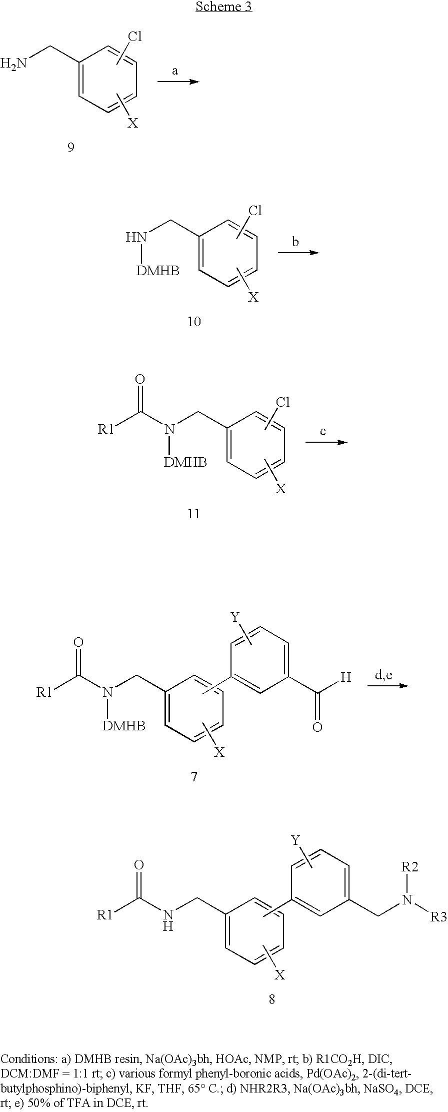

### Example 216

### Preparation of 3-cyano-N-{[6-fluoro-2′-(methyloxy)-5′-(1-piperazinylmethyl)-3-biphenylyl]methyl}benzamide

To a 50 mL shaker vessel was added 2,6-dimethoxy-4-polystyrenebenzyloxy-benzaldehyde (DMHB resin) (2 g, 1.5 mmol/g, 3 mmol) and 25 mL of NMP. 3-Chloro-4-fluorobenzylamine (1.92 g, 12 mmol), HOAC (2.5 mL, 10%), and Na(OAc)₃BH (3.18 g, 15 mmol) were then added. The mixture was shaken at rt for overnight. The resulting resin was washed with NMP (25 mL×2), DCM (25 mL×2), MeOH (25 mL×2) and DCM (25 mL×2) and dried in vacuum oven at 35° C. for overnight to yield DMHB resin-bound 3-chloro-4-fluorobenzylamine.

To a mixture of the above resin (0.07 g, 1.2 mmol/g (theoretical loading), 0.084 mmol) in DCE:DMF (1:1, 3 mL) was added 3-cyanobenzoic acid (0.124 g, 0.84 mmol) and DIC (131 uL, 0.84 mmol). The mixture was shaken at rt for overnight. The resulting resin was washed with DMF (2 mL×2), DCM (2 mL×2), MeOH (2 mL×2) and DCM (2 mL×2), and dried in vacuum oven at 35° C. for overnight. An analytical amount of the resin was cleaved with 50% of TFA in DCE for 30 min. The resulting solution was concentrated in vacuo and dissolved in 0.5 mL of MeOH. MS (ESI): 289 [M+H]⁺.

To a mixture of the above resin-bound N-[(3-chloro-4-fluorophenyl)methyl]-3-cyanobenzamide (0.081 g, 1.04 mmol/g (theoretical loading), 0.084 mmol) in 3 mL of THF was added Pd(OAc)₂ (0.015 g, 0.0672 mmol), 2-(di-tert-butylphosphino)biphenyl (0.040 g, 0.134 mmol), (5-formyl-2-methoxyphenyl)boronic acid (0.181 g, 1.01 mmol) and potassium fluoride (0.117 g, 2.016 mmol). The resulting mixture was purged with argon for 10 min and was then shaken at 65° C. for 16 h. The resin was washed with THF (2 mL×2), THF:H₂O (1:1, 2 mL×2), H₂O (2 mL×2), THF:H₂O (1:1, 2 mL×2), THF (2 mL×2), DCM (2 mL×2), and dried in vacuum oven at 35° C. for overnight. An analytical amount of the resin was cleaved with 50% of TFA in DCE for 30 min. The resulting solution was concentrated in vacuo and dissolved in 0.5 mL of CH₃CN. MS (ESI): 389 [M+H]⁺.

To a mixture of the above resin-bound 3-cyano-N-{[6-fluoro-5′-formyl-2′-(methyloxy)-3-biphenylyl]methyl}benzamide (0.079 g, 0.94 mmol/g (theoretical loading), 0.084 mmol) in DCE (4 mL) was added Na₂SO₄ (0.06 g, 0.42 mmol) and 1,1-dimethylethyl 1-piperazinecarboxylate (0.078 g, 0.42 mmol). After shaking for 10 min, Na(OAc)₃BH (0.107 g, 0.504 mmol) was added. The mixture was shaken at rt for overnight. The resulting resin was washed with THF (2 mL×2), THF:H₂O (1:1,2 mL×2), H₂O (2 mL×2), THF:H₂O (1:1, 2 mL×2), THF (2 mL×2), DCM (2 mL×2), and dried in vacuum oven at 35° C. for overnight. The resin was cleaved with 2 mL of 50% of TFA in DCE for 30 min and treated again with 2 mL of 50% of TFA in DCE for 30 min. The combined cleavage solution was concentrated in vacuo. The residue was dissolved in DMSO and purified using a Gilson semi-preparative HPLC system with a YMC ODS-A (C-18) column 50 mm by 20 mm ID, eluting with 10% B to 90% B in 3.2 min, hold for 1 min where A=H₂O (0.1% trifluoroacetic acid) and B=CH₃CN (0.1% trifluoroacetic acid) pumped at 25 mL/min, to produce 3-cyano-N-{[6-fluoro-2′-(methyloxy)-5′-(1-piperazinylmethyl)-3-biphenylyl]methyl}benzamide as a bis-trifluoroacetate salt (white powder, 2.3 mg, 6% over 5 steps). MS (ESI): 459 [M+H]⁺.

Proceeding in a similar manner, but replacing 3-cyanobenzoic acid with the appropriate acids, and/or replacing 1,1-dimethylethyl 1-piperazinecarboxylate with the appropriate amines, and/or replacing 3-chloro-4-fluorobenzylamine with appropriate chlorobenzylamines, and/or replacing 5-formyl-2-methoxyphenylboronic acid with appropriate formylphenyl boronic acids, the compounds listed in Tables 14-17 were prepared.

TABLE 14

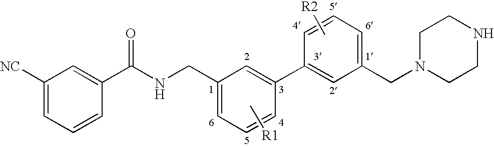

| | | | MS |
|---|---|---|---|
| Example | R1 | R2 | [M + H]⁺ |
| 217 | 4-fluoro | 4′-methoxy | 459 |
| 218 | 4-fluoro | 6′-fluoro | 447 |
| 219 | 4-fluoro | 6′-methoxy | 459 |
| 220 | 4-methoxy | hydrogen | 441 |
| 221 | 4-methoxy | 4′-methoxy | 471 |
| 222 | 4-methoxy | 6′-fluoro | 459 |
| 223 | 4-methoxy | 6′-methoxy | 471 |
| 224 | 6-methyl | hydrogen | 425 |
| 225 | 6-fluoro | hydrogen | 429 |
| 226 | 6-fluoro | 4′-methoxy | 459 |
| 227 | 6-fluoro | 6′-fluoro | 447 |
| 228 | 6-fluoro | 6′-methoxy | 459 |
| 229 | 2-fluoro | hydrogen | 429 |

TABLE 15

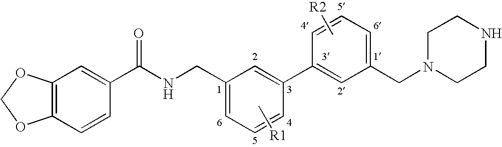

| | | | MS |
|---|---|---|---|
| Example | R1 | R2 | [M + H]⁺ |
| 230 | 4-fluoro | hydrogen | 448 |
| 231 | 4-fluoro | 4′-methoxy | 478 |
| 232 | 4-fluoro | 6′-fluoro | 466 |
| 233 | 4-fluoro | 6′-methoxy | 478 |
| 234 | 4-methoxy | hydrogen | 460 |
| 235 | 4-methoxy | 6′-fluoro | 459 |
| 236 | 4-methoxy | 6′-methoxy | 490 |
| 237 | 6-methyl | hydrogen | 444 |
| 238 | 6-fluoro | hydrogen | 448 |
| 239 | 2-fluoro | hydrogen | 448 |
| 240 | 2-fluoro | 6′-methoxy | 478 |

TABLE 16

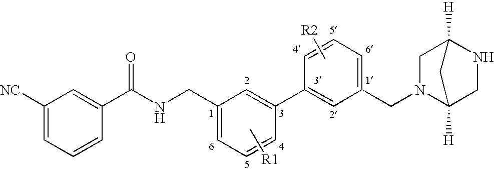

| | | | MS |
|---|---|---|---|
| Example | R1 | R2 | [M + H]⁺ |
| 241 | 4-fluoro | hydrogen | 441 |
| 242 | 4-methoxy | hydrogen | 453 |
| 243 | 6-fluoro | hydrogen | 441 |
| 244 | 4-fluoro | 6′-fluoro | 459 |
| 245 | 4-methoxy | 4′-methoxy | 483 |
| 246 | 4-methoxy | 6′-fluoro | 471 |
| 247 | 4-methoxy | 6′-methoxy | 483 |
| 248 | 6-fluoro | 4′-methoxy | 471 |
| 249 | 6-fluoro | 6′-fluoro | 549 |
| 250 | 6-fluoro | 6′-methoxy | 471 |

TABLE 17

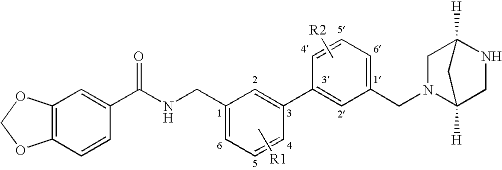

| | | | MS |
|---|---|---|---|
| Example | R1 | R2 | [M + H]⁺ |
| 251 | 4-fluoro | hydrogen | 460 |
| 252 | 4-methoxy | hydrogen | 472 |
| 253 | 6-methyl | hydrogen | 456 |
| 254 | 6-fluoro | hydrogen | 460 |
| 255 | 2-fluoro | hydrogen | 460 |
| 256 | 4-fluoro | 4′-methoxy | 490 |
| 257 | 4-fluoro | 6′-fluoro | 478 |
| 258 | 4-methoxy | 6′-fluoro | 490 |
| 259 | 6-fluoro | 4′-methoxy | 490 |

### Preparation 4

Resin-bound bromo benzylamides 6 underwent Suzuki coupling with dihydroxyboranyl benzoic acids to give biaryl acids 12 (Scheme 4). Amide formation of 12 with amines, followed by cleavage, yielded the desired biaryl amides 13.

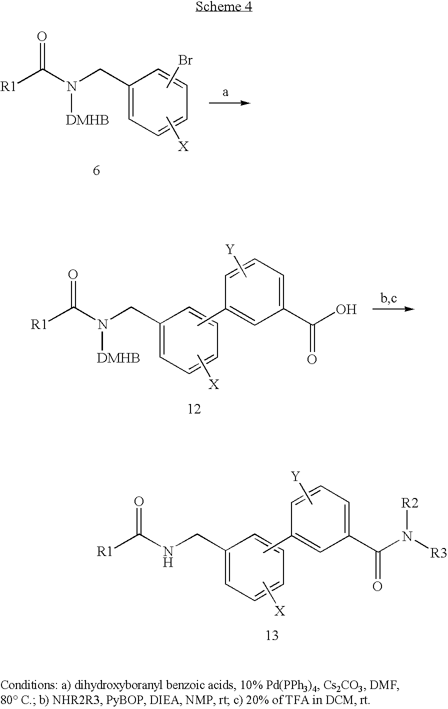

### Example 260

### Preparation of N-{[3′-(1-piperazinylcarbonyl)-3-biphenylyl]methyl}-1,3-benzodioxole-5-carboxamide

To a mixture of DMHB resin-bound N-[(3-bromophenyl)methyl]-1,3-benzodioxole-5-carboxamide (36a, 1.3 g, 1.0 mmol/g (theoretical loading), 1.3 mmol) in 30 mL of DMF was added 3-(dihydroxyboranyl)benzoic acid (1.3 g, 7.8 mmol), 2 M CSCO₃ aqueous solution (1.95 mL, 3.9 mmol), and Pd(PPh₃)₄ (0.15 g, 0.13 mmol). The mixture was purged with argon for 5 min and was then heated at 80° C. for overnight. The resin was washed with DMF (50 mL), THF (50 mL×2), THF:H₂O (1:1, 50 mL×2), H₂O (50 mL×2), THF:H₂O (1:1, 50 mL×2), THF (50 mL×2), DCM (50 mL×2), and dried in vacuum oven at 35° C. for overnight. An analytical amount of the resin was cleaved with 20% of TFA in DCM for 10 min. The resulting solution was concentrated in vacuo and dissolved in 0.5 mL of MeOH. MS (ESI): 376 [M+H]⁺.

To a mixture of the above resin bound 3′-{[(1,3-benzodioxol-5-ylcarbonyl)amino]methyl}-3-biphenylcarboxylic acid (80 mg, 0.97 mmol/g (theoretical loading), 0.078 mmol) in 2.5 mL of NMP was added 1,1-dimethylethyl 1-piperazinecarboxylate (0.14 g, 0.75 mmol), DIEA (0.13 mL, 0.75 mmol), and PyBOP (0.2 g, 0.376 mmol). The mixture was shaken at rt for overnight. The resin was washed with NMP (10 mL×2), DCM (10 mL×2), MeOH (10 mL×2), DCM (10 mL×2), and dried in vacuum oven at 35° C. for overnight. The resin was cleaved with 2 mL of 20% of TFA in DCE for 30 min and treated again with 2 mL of 20% of TFA in DCE for 30 min. The combined cleavage solution was concentrated in vacuo. The residue was dissolved in DMSO and purified using a Gilson semi-preparative HPLC system with a YMC ODS-A (C-18) column 50 mm by 20 mm ID, eluting with 10% B to 90% B in 3.2 min, hold for 1 min where A=H₂O (0.1% trifluoroacetic acid) and B=CH₃CN (0.1% trifluoroacetic acid) pumped at 25 mL/min, to produce N-{[3′-(1-piperazinylcarbonyl)-3-biphenylyl]methyl}-1,3-benzodioxole-5-carboxamide as a mono-trifluoroacetate salt (white powder, 8.5 mg, 25% over 5 steps). MS (ESI): 444 [M+H]⁺.

Proceeding in a similar manner, but replacing 1,1-dimethylethyl 1-piperazinecarboxylate with the appropriate amines, the compounds listed in Table 18 were prepared.

TABLE 18

| | | MS |
|---|---|---|
| Example | Compound | [M + H]⁺ |
| 261 | 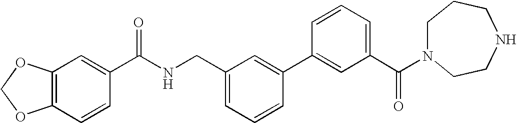 | 458 |
| 262 | 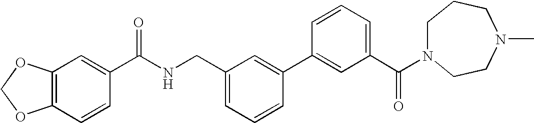 | 472 |
| 263 | 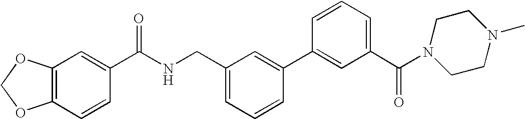 | 458 |

### Preparation 5

1-(3-Bromobenzyl)piperazine (15) was loaded on activated Wang-resin 14 to form resin-bound bromide 16 which upon Suzuki coupling with 3-formyl benzeneboronic acid gave aldehyde 17 (Scheme 5). Reductive alkylation of 17 with primary amines afforded benzylamines 18. Amide formation with acid chlorides, followed by resin cleavage, yielded N-alkylated benzylamides 19.

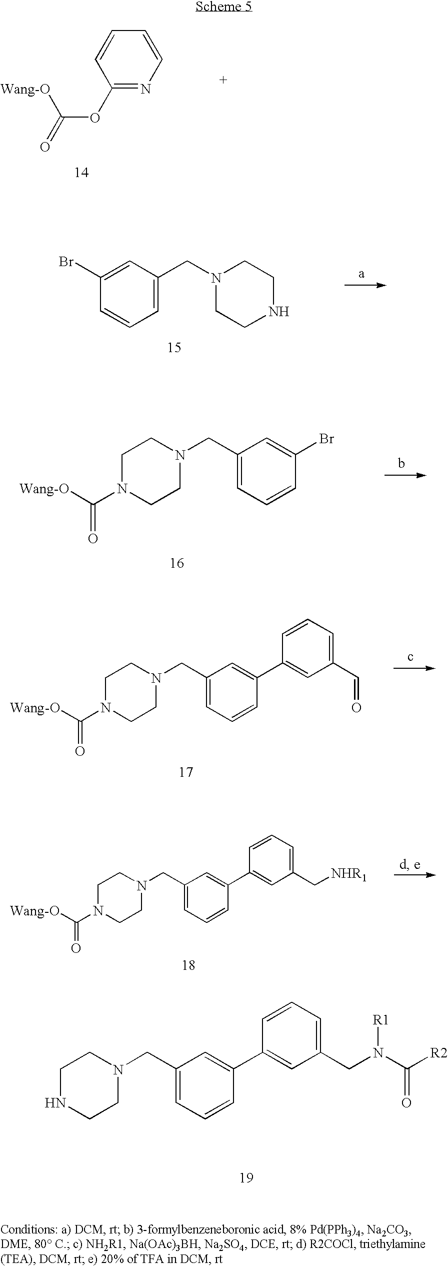

### Example 264

### Preparation of N-cyclopropyl-N-{[3′-(1-piperazinylmethyl)-3-biphenylyl]methyl}octanamide

To a solution of N-Boc-piperazine (18.6 g, 0.1 mol) in dry dichloromethane (250 mL) was added with stirring 3-bromobenzaldehyde (19.43 g, 0.105 mol). After stirring under argon for 30 min, acetic acid (6.3 g, 0.105 mol) was added followed by solid sodium triacetoxyborohydride (25.4 g, 0.12 mol) portionwise over 20 min. to prevent excess warming and effervescence. The mixture was then stirred under argon for 18 h. Saturated NaHCO₃ solution was added cautiously with stirring until effervescence ceased, and the organic phase was separated, washed with NaHCO₃ solution and brine, then dried (MgSO₄) and evaporated in vacuo.

The oily product was dissolved in DCM (135 mL), and water (5 mL) was added. The solution was stirred whilst adding trifluoroacetic acid (70 mL) portionwise with caution. Stirring was continued for 3.5 h, and the solution was then evaporated in vacuo. The residue was redissolved in DCM and stirred with saturated NaHCO₃ solution until effervescence ceased, and more NaHCO₃ solution was added until the solution became basic. The organic phase was separated, washed with NaHCO₃ solution, 2 M NaOH solution and brine, dried (MgSO₄) and evaporated in vacuo to produce 1-(3-Bromobenzyl)piperazine (15) as a pale brown oil (21.4 g, 84% over 2 steps); ¹H NMR, δ (CDCl₃) 1.84 (br s), 2.41 and 2.89 (each 4H, m) 3.45 (2H, s) 7.10-7.49 (4H, m). MS (ESI), 255 [M+H]⁺.

Wang resin (15.9 g, 1.7 mmol.g⁻¹, 27 mmol) was suspended in anhydrous DCM and di-2-pyridylcarbonate and triethylamine were added. The mixture was shaken overnight under argon. The resin was filtered and washed 4 times with DCM then dried at room temperature in vacuo and used without further characterization.

Wang 2-pyridyl carbonate resin (14, 80 mmol) obtained from above was suspended in dry DCM (400 mL) and a solution of 1-(3-bromobenzyl)piperazine (15, 40.8 g, 160 mmol) in DCM (200 mL) was added. The mixture was shaken under argon for 24 h. The resin was filtered, washed with DCM (300 mL×3), THF (300 mL×3), DCM (300 mL×3), and ether (300 mL). The product resin was dried in vacuo. A sample of the resin (16, 50 mg) was shaken with trifluoroacetic acid (0.2 mL) and DCM (0.8 mL) for 2 h. The resin was filtered and washed with DCM and methanol, and the filtrate evaporated to give the bis-trifluoroacetate salt of the amine 15 (26 mg, 93%); ¹H NMR, δ (CD₃OD) 3.05 (4H, m), 3.37 (4H, m), 3.95 (2H, s), 7.35 (2H, m), 7.54 (1H, dd, J1.5 and 6.2 Hz), 7.64 (1H, d, J=1.5 Hz). MS (ESI), 255 [M+H]⁺.

The above resin 16 (22.0 g, 25.3 mmol) was suspended in 1,2-dimethoxyethane (DME) (500 mL) in a 3-neck 2 L flask fitted with an overhead stirrer. Argon was bubbled through the mixture for 30 min before adding tetrakis(triphenylphosphine) palladium (2.34 g, 2.03 mmol). 3-Formylbenzeneboronic acid (11.4 g, 76 mmol) was added followed by more DME (190 mL). A solution of Na₂CO₃ (16.1 g, 152 mmol) in water (76 mL) was then added and the mixture heated to 80° C., whilst stirring under an argon atmosphere. After 16 h, the reaction mixture was cooled and the black resin product was filtered and washed with THF (500 mL), water (3×500 mL), THF:water (1:1, 2×500 mL), THF (3×500 mL), DCM (3×500 mL) and ether (2×500 mL). It was then dried at 40° C. in vacuo to afford product resin 17 (23.4 g).

The reductive alkylation reaction was performed in IRORI™ kans in a combinatorial process. The formyl resin 17 (30 mg) was placed in a kan containing a radiofrequency tag. In a mixture with other kans containing formyl resins, the kan was placed in a flask with 1,2-dichloroethane (1 mL/kan) and vacuum was applied and released to ensure that solvent filled the kan. 5 Equivalents each of sodium sulfate, cyclopropylamine and acetic acid were added to the flask which was purged with argon and then shaken for 3 h. Solid sodium triacetoxyborohydride was then added and shaking continued for a further 22 h. The kans were filtered and washed with THF, THF-water (1:1), water (×2), THF-water (1:1), THF, water, DMF, methanol, THF (×3) and DCM (×3), and then dried in vacuo at 40° C. The kan containing resin bound product 18 (R1=cyclopropyl) was identified by reading the radiofrequency tag.

The kan containing resin product 18 (R1=cyclopropyl) was reacted combinatorially in a mixture with kans containing other related amine resins. The kan was suspended in dry DCM (1 mL/kan) and vacuum applied and released to ensure filling of the kan with solvent. Triethylamine (12 eq) and octanoyl chloride (10 eq) were added and the mixture was shaken for 22 h. The kans were filtered and washed with DCM (×2), THF, THF:water (1:1), THF (×2) and DCM (×3), and then dried at 40° C. in vacuo. The kan containing the resin-bound product 19 (R1=cyclopropyl, R2=heptyl) was identified by reading the radiofrequency tag.

The kan containing the resin-bound product 19 (R1=cyclopropyl, R2=heptyl) was placed in a well of a cleavage block, and treated with a solution of 20% trifluoroacetic acid, 3% water, 77% DCM (2 mL). The block was gently agitated for 2 h, and the solution drained into a vial. The kan was washed with DCM:methanol (1:1, 1 mL), and the solution again drained into the vial. The solution in the vial was evaporated in a Genevac to produce N-cyclopropyl-N-{[3′-(1-piperazinylmethyl)-3-biphenylyl]methyl}octanamide as a bis-trifluoroacetate salt (7.6 mg, 28% over 5 steps). MS (ESI), 448 [M+H]⁺.

Proceeding in a similar manner, but replacing cyclopropylamine with the appropriate amines, and/or replacing octanoyl chloride with the appropriate acid chlorides, the compounds listed in Table 19 were prepared.

TABLE 19

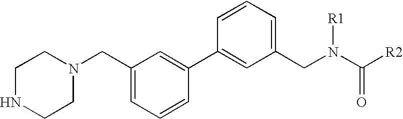

| | | | MS |
|---|---|---|---|
| Example | R1 | R2 | [M + H]⁺ |
| 265 | 2-(1-pyrrolidinyl)-ethyl | 1-heptyl | 505 |
| 266 | 2-(4-morpholinyl)-ethyl | 1-heptyl | 521 |
| 267 | 3-pyridinyl-methyl | 1-heptyl | 499 |
| 268 | 1-(phenylmethyl)-4- | 1-heptyl | 581 |
| | piperidinyl | | |
| 269 | benzyl | 1-heptyl | 498 |
| 270 | 3,4-dichloro-benzyl | 1-heptyl | 566 |
| 271 | 4-methoxy-benzyl | 1-heptyl | 528 |
| 272 | 2-(N,N-dimethylamino)- | 1-heptyl | 479 |
| | ethyl | | |
| 273 | 3-phenylprop-1-yl | 1-heptyl | 526 |
| 274 | 3-(4-methyl-1-piperazinyl)- | 1-heptyl | 548 |
| | prop-1-yl | | |
| 275 | 3-(methyloxy)-prop-1-yl | 1-heptyl | 480 |
| 276 | 2-(2-thiophenyl)-ethyl | 1-heptyl | 518 |
| 277 | 3-(hydroxy)-3-oxo-prop-1- | 1-heptyl | 480 |
| | yl | | |
| 278 | 2-bromo-benzyl | 1-heptyl | 576 |
| 279 | tetrahydro-2-furanyl-methyl | 1-heptyl | 492 |
| 280 | cyclopropyl | 3,4-methylenedioxy- | 470 |
| | | phenyl | |
| 281 | 2-( 1-pyrrolidinyl)-ethyl | 3,4-methylenedioxy- | 527 |
| | | phenyl | |
| 282 | 1-naphthyl-methyl | 3,4-methylenedioxy- | 570 |
| | | phenyl | |
| 283 | 2-(4-morpholinyl)-ethyl | 3,4-methylenedioxy- | 543 |
| | | phenyl | |
| 284 | 3-pyridinyl-methyl | 3,4-methylenedioxy- | 521 |
| | | phenyl | |
| 285 | 3-(trifluoromethyl)-benzyl | 3,4-methylenedioxy- | 588 |
| | | phenyl | |
| 286 | 2-(N,N-dimethylamino)- | 3,4-methylenedioxy- | 501 |
| | ethyl | phenyl | |
| 287 | 1-butyl | 3,4-methylenedioxy- | 486 |
| | | phenyl | |
| 288 | 3-(4-methyl-1-piperazinyl)- | 3,4-methylenedioxy- | 570 |
| | prop-1-yl | phenyl | |
| 289 | 3-(methyloxy)-prop-1-yl | 3,4-methylenedioxy- | 502 |
| | | phenyl | |
| 290 | 3-(hydroxy)-3-oxo-prop-1- | 3,4-methylenedioxy- | 502 |
| | yl | phenyl | |
| 291 | 2-bromo-benzyl | 3,4-methylenedioxy- | 598 |
| | | phenyl | |
| 292 | 3-methoxy-phenethyl | 3,4-methylenedioxy- | 564 |
| | | phenyl | |
| 293 | tetrahydro-2-furanyl-methyl | 3,4-methylenedioxy- | 514 |
| | | phenyl | |

### Preparation 6

The 4-fluoro-derivatives of general structure 24 were prepared in solution phase following the route outlined in Scheme 6. Firstly, the boronic acid 20 underwent a Suzuki palladium coupling with the bromide 21 to give the 4-fluoro-biphenyl derivative 22. Further reduction of the nitrile moiety with borane yielded the primary amine 23. Subsequent coupling of 23 to the appropriate benzoic acids gave the respective products 24.

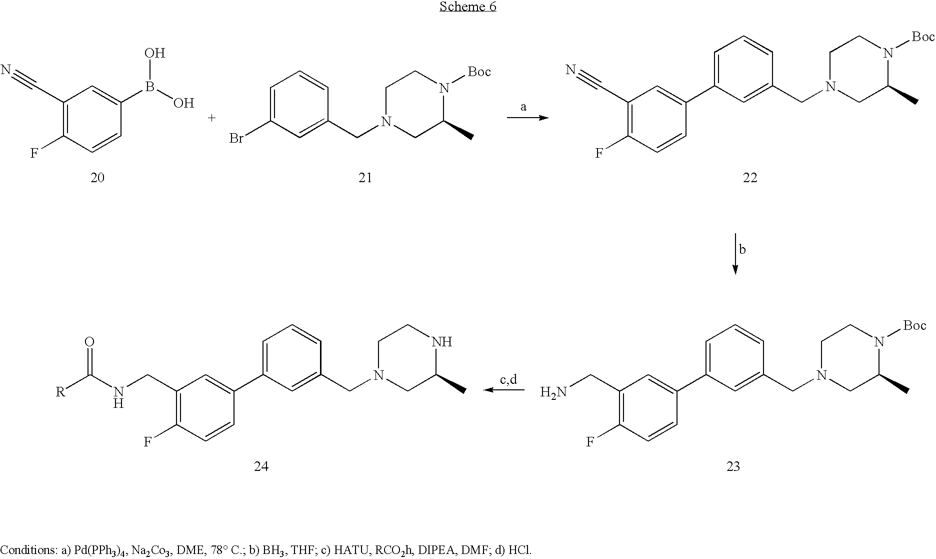

### Example 294

### Preparation of 3-cyano-N-[(4-fluoro-3′-{[(3S)-3-methyl-1-piperazinyl]methyl}-3-biphenylyl)methyl]benzamide

### 1,1-Dimethylethyl (2S)-4-[(3′-cyano-4′-fluoro-3-biphenylyl)methyl]-2-methyl-1-piperazinecarboxylate

To a solution of (3-cyano-4-fluorophenyl)boronic acid (0.983 g, 5.96 mmol) in DME (40 mL) was added 1,1-dimethylethyl (2S)-4-[(3-bromophenyl)methyl]-2-methyl-1-piperazinecarboxylate (2.20 g, 5.96 mmol) followed by Na₂CO₃ (17 mL, 2M in H₂O, 34.0 mmol). The reaction vessel was flushed with argon, and tetrakis(triphenylphosphine)palladium(0) (2.06 g, 1.78 mmol) was added. The reaction mixture was placed in an oil bath at 78° C. under argon for overnight. The reaction was diluted with EtOAc (600 mL) and washed with H₂O (250 mL). The water layer was extracted with EtOAc (1×100 mL). The combined organic layers were dried over MgSO₄, filtered, and concentrated under vacuum. Purification of the crude residue by flash chromatography (20% EtOAc/80% hexane) on silica gel gave the title compound (1.77 g, 73.1%). EI-MS m/z 410 (M−H)⁺.

### 1,1-Dimethylethyl (2S)-4-{[3′-(aminomethyl)-4′-fluoro-3-biphenylyl]methyl}-2-methyl-1-piperazinecarboxylate

A solution of 1,1-dimethylethyl (2S)-4-[(3′-cyano-4′-fluoro-3-biphenylyl)methyl]-2-methyl-1-piperazinecarboxylate (2.29 g, 5.59 mmol) in THF (50 mL) was flushed with argon. Borane (19 mL, 1M in THF, 19 mmol) was slowly added and the reaction was allowed to stir at room temperature overnight. The reaction was quenched slowly with water, diluted with water (175 mL) and then extracted with EtOAc (2×250 mL). The combined organic layers were dried over MgSO₄, filtered, and concentrated under vacuum. The crude residue was placed onto a SPE silica cartridge (20 g) using 50% hexane/50% EtOAc, and then eluted with the following sequence: 50% hexane/50% EtOAc, 10% MeOH/90% DCM, 30% MeOH/70% DCM. The product fractions were combined and concentrated to give the title compound (1.48 g, 64.1%). EI-MS m/z 414 (M−H)⁺.

### General Procedure for Amide Formation and Boc Deprotection

### 3-cyano-N-[(4-fluoro-3′-{[(3S)-3-methyl-1-piperazinyl]methyl}-3-biphenylyl)methyl]benzamide

To a solution of 1,1-dimethylethyl (2S)-4-{[3′-(aminomethyl)-4′-fluoro-3-biphenylyl]methyl}-2-methyl-1-piperazinecarboxylate (0.100 g, 0.242 mmol) in DMF (2.5 mL) were added 3-cyanobenzoic acid (0.038 g, 0.260 mmol), HATU (0.102 g, 0.268 mmol), and diisopropylethylamine (0.10 mL, 0.574 mmol). The reaction was allowed to stir at room temperature for 2 days. The reaction was diluted with EtOAc (75 mL), washed with 1N HCl (2×20 mL), saturated NaHCO₃ (3×20 mL), then brine (2×20 mL). The organic layer was dried over MgSO₄, filtered, and concentrated under vacuum. The residue was taken up in MeOH (4 mL) and HCl (4N in 1,2-dioxane, 2.5 mL) was added. The reaction was allowed to stir at room temperature overnight. The reaction was concentrated under vacuum, and the residue was taken up in 1 mL DMSO/1 mL MeOH and purified via MDAP (10-90% CH₃CN/H₂O/(0.1% TFA)). The desired fractions were isolated, and then taken up in DCM (8 mL) and 1N NaOH (8 mL) and allowed to stir for 1 hour. The DCM was isolated using a phase separator and then concentrated under vacuum to give the title compound (96 mg, 90%). EI-MS m/z 443 (M−H)⁺.

### Example 295

### Preparation of N-[(4-fluoro-3′-{[(3S)-3-methyl-1-piperazinyl]methyl}-3-biphenylyl)methyl]-3-(phenylcarbonyl)benzamide

Following the general procedure outlined in Example 294, 1,1-dimethylethyl (2S)-4-{[3′-(aminomethyl)-4′-fluoro-3-biphenylyl]methyl}-2-methyl-1-piperazinecarboxylate (0.095 g, 0.231 mmol), 3-(phenylcarbonyl)benzoic acid (0.058 g, 0.255 mmol), HATU (0.102 g, 0.268 mmol), and diisopropylethylamine (0.10 mL, 0.574 mmol) in DMF (2.5 mL) were reacted to give the desired product (0.045 g, 37.4%). EI-MS m/z 522 (M−H)⁺.

### Example 296

### Preparation of N-[(4-fluoro-3′-{[(3S)-3-methyl-1-piperazinyl]methyl}-3-biphenylyl)methyl]-1,3-benzodioxole-5-carboxamide

Following the general procedure outlined in Example 294, 1,1-dimethylethyl (2S)-4-{[3′-(aminomethyl)-4′-fluoro-3-biphenylyl]methyl}-2-methyl-1-piperazinecarboxylate (0.098 g, 0.235 mmol), 1,3-benzodioxole-5-carboxylic acid (0.039 g, 0.235 mmol), HATU (0.107 g, 0.280 mmol), and diisopropylethylamine (0.10 mL, 0.574 mmol) in DMF (2.5 mL) were reacted to give the desired product (0.043 g, 40.1%). EI-MS m/z 462 (M−H)⁺.

### Example 297

### Preparation of 3-(ethyloxy)-N-[(4-fluoro-3′-{[(3S)-3-methyl-1-piperazinyl]methyl}-3-biphenylyl)methyl]benzamide

Following the general procedure outlined in Example 294, 1,1-dimethylethyl (2S)-4-{[3′-(aminomethyl)-4′-fluoro-3-biphenylyl]methyl}-2-methyl-1-piperazinecarboxylate (0.099 g, 0.240 mmol), 3-(ethyloxy)benzoic acid (0.042 g, 0.253 mmol), HATU (0.103 g, 0.271 mmol), and diisopropylethylamine (0.10 mL, 0.574 mmol) in DMF (2.5 mL) were reacted to give the desired product (0.037 g, 33.7%). EI-MS m/z 462 (M−H)⁺.

### Example 298

### Preparation of 3-acetyl-N-[(4-fluoro-3′-{[(3S)-3-methyl-1-piperazinyl]methyl}-3-biphenylyl)methyl]benzamide

Following the general procedure outlined in Example 294, 1,1-dimethylethyl (2S)-4-{[3′-(aminomethyl)-4′-fluoro-3-biphenylyl]methyl}-2-methyl-1-piperazinecarboxylate (0.100 g, 0.242 mmol), 3-acetylbenzoic acid (0.044 g, 0.269 mmol), HATU (0.104 g, 0.274 mmol), and diisopropylethylamine (0.10 mL, 0.574 mmol) in DMF (2.5 mL) were reacted to give the desired product (0.039 g, 35.4%). EI-MS m/z 460 (M−H)⁺.

### Example 299

### Preparation of 3-[(3,4-dichlorophenyl)carbonyl]-N-[(4-fluoro-3′-{[(3S)-3-methyl-1-piperazinyl]methyl}-3-biphenylyl)methyl]benzamide

Following the general procedure outlined in Example 294, 1,1-dimethylethyl (2S)-4-{[3′-(aminomethyl)-4′-fluoro-3-biphenylyl]methyl}-2-methyl-1-piperazinecarboxylate (0.108 g, 0.261 mmol), 3-[(3,4-dichlorophenyl)carbonyl]benzoic acid (0.074 g, 0.251 mmol), HATU (0.110 g, 0.290 mmol), and diisopropylethylamine (0.10 mL, 0.574 mmol) in DMF (2.5 mL) were reacted to give the desired product (0.1095 g, 76.0%). EI-MS m/z 590 (M−H)⁺.

### Preparation 7

Scheme 7 outlines a solution phase route to synthesize compounds with structure 31. Reductive amination of the benzaldehyde 25 with the BOC-protected piperazine 26 gave the tertiary amine 27. Boronation using trimethyl borate led to the boronic acid 28. Further Suzuki coupling of 28 with the commercially available bromide 29 produced compound 30, which in turn could be coupled with the appropriate carbocylic acid R₁CO₂H or acyl halide and deptrotected to furnish the products 31.

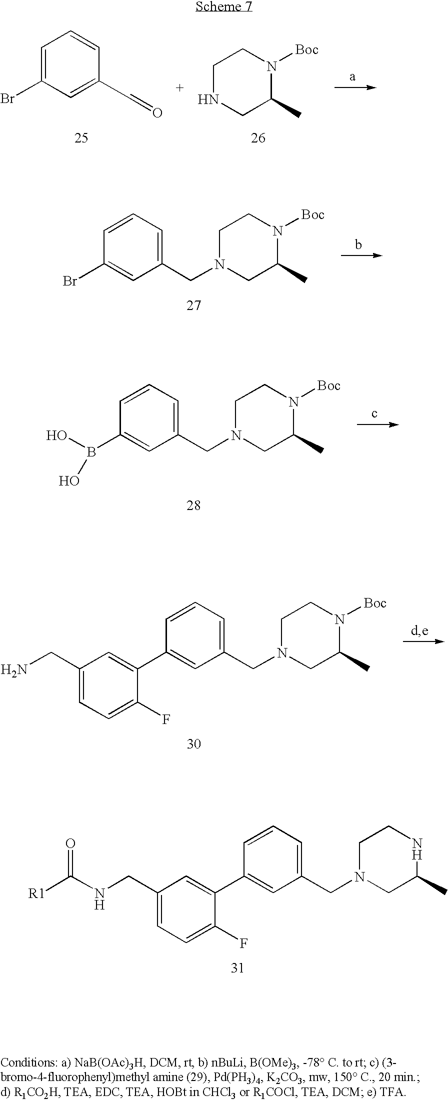

### Intermediate 26

### 1,1-dimethylethyl (2S)-2-methyl-1-piperazinecarboxylate

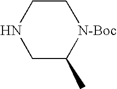

A solution of (S)-2-methyl piperazine (2 g, 20 mmol) in THF (200 mL) was mixed with n-BuLi (25 mL, 1.6 M in hexane, 40 mmol) at rt. The solution was stirred for 30 min before TBDMSCl (3.04 g, 20 mmol) was added. The mixture was stirred for an additional hour and (BoC)₂O (5.2 g, 24 mmol) was added to the solution. The resulting mixture was stirred for another hour and diluted with H₂O (50 mL). The organic layer was separated, washed with brine (50 mL), dried over Na₂SO₄ and concentrated under vacuum. Flash chromatography on silica (5% MeOH/2% NH₄OH/93% CH₂Cl₂) then provided the title compound as a yellow oil (3.7 g, 93%). LC/MS: m/z, 201 (M+H); ¹HNMR (CDCl₃) 1.26 (3H, d), 1.49 (9H, s), 2.1 (1H, s), 2.7 (1H, m), 2.85 (1H, m), 3.0 (3H, m), 3.8 (1H, m), 4.2 (1H, m).

### Intermediate 27

### 1,1-dimethylethyl (2S)-4-[(3-bromophenyl)methyl]-2-methyl-1-piperazine carboxylate

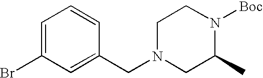

A solution of 1,1-dimethylethyl (2S)-2-methyl-1-piperazinecarboxylate (Intermediate 26, 100 mg, 0.5 mmol) in CH₂Cl₂ (5 mL) was mixed with 3-bromo benzaldehyde (0.06 mL, 0.5 mmol) and NaB(OAc)₃H (0.16 g, 0.75 mmol). The resulting mixture was stirred for 12 hours, diluted with dichloromethane (30 mL) and washed with brine (50 mL). The organic layer was collected, dried over Na₂SO₄ and concentrated. Separation via a combiflash system then afforded the title compound (150 mg, 81%). LC/MS: m/z, 369 (M+H); ¹HNMR (MeOD) 1.26 (3H, d), 1.47 (9H, s), 2.0 (1H, m), 2.1 (1H, m), 2.6 (1H, m), 2.8 (1H, m), 3.1 (1H, m), 3.3 (2H, s), 3.4 (1H, m), 3.5 (1H, m), 3.8 (1H, m), 4.2 (1H, m), 4.88 (1H, s), 7.25 (1H, m), 7.3 (1H, m), 7.4 (1H, m), 7.55 (1H, s).

### Intermediate 28

### {3-[((3S)-4-{[(1,1-dimethylethyl)oxy]carbonyl}-3-methyl-1-piperazinyl)methyl]phenyl}boronic acid

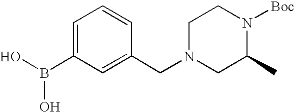

A solution of 1,1-dimethylethyl (2S)-4-[(3-bromophenyl)methyl]-2-methyl-1-piperazine carboxylate (Intermediate 27, 1.8 g, 4.9 mmol) in THF (4.9 mL) was mixed with n-BuLi (3.7 mL, 1.6 M in Hexane, 5.9 mmol) at −78° C. and stirred for 30 min before B(OMe)₃ (2.2 mL, 19.6 mmol) was added. After addition, the resulting solution was warmed up to room temperature within 2 hours. The mixture was then mixed with saturated aqueous NH₄Cl solution (10 mL), stirred for 25 minutes at room temperature, diluted with H₂O (5 mL) and extracted with Et₂O (2×30 mL). The organic layers were combined, dried over Na₂SO₄, filtered and concentrated to afford the crude title compound (1.7 g, quantitative yield). LC/MS: m/z 335 (M+H); ¹H-NMR (MeOD) δ 1.24 (d, 3H), 1.46 (s, 9H), 2.00 (m, 1H), 2.13 (m, 1H), 2.68 (d, 1H), 2.82 (d, 1H), 3.12 (m, 1H), 3.44 (m, 1H), 3.56 (m, 1H), 3.80 (d, 1H), 4.18 (m, 1H), 7.33 (m, 1H), 7.38 (m, 1H), 7.51 (d, 1H), 7.59 (s, 1H).

### Intermediate 30

### 1,1-dimethylethyl(2S)-4-{[5′-(aminomethyl)-2′-fluoro-3-biphenylyl]methyl}-2-methyl-1-piperazinecarboxylate

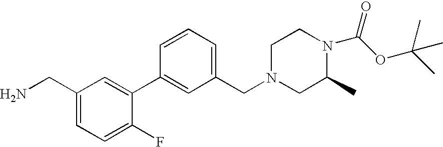

To a solution of [(3-bromo-4-fluorophenyl)methyl]amine hydrochloride (1.68 g, 7 mmol) in dioxane/H₂O (10 mL/3.3 mL) were added {3-[((3S)-4-{[(1,1-dimethylethyl)oxy]carbonyl}-3-methyl-1-piperazinyl)methyl]phenyl}boronic acid (intermediate 28, 2.33 g, 7 mmol), K₂CO₃ (4.83, 35 mmol) and Pd(PPh₃)₄ (405 mg, 0.35 mmol). The resulting mixture was heated at 150° C. in a pressure vessel for 2 hours, then cooled to rt and diluted with EtOAc (50 mL). The organic layer was collected and the aqueous layer was extracted by EtOAc (30 mL). The organic layers were combined, dried over Na₂SO₄, filtered and concentrated. The residue was purified by Gilson preparatory HPLC, eluting with acetonitrile/water/0.1% TFA (10/90 to 90/10, v/v, over 12 min), to give the title compound (1.08 g, 37%). LC/MS: m/z, 414 (M+H), 1.83 min.

### Example 300

### N-[(6-fluoro-3′-{[(3S)-3-methyl-1-piperazinyl]methyl}-3-biphenylyl)methyl]benzamide

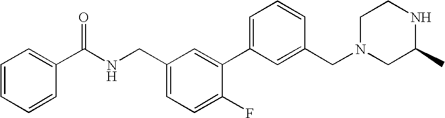

To a solution of 1,1-dimethylethyl (2S)-4-{[5′-(aminomethyl)-2′-fluoro-3-biphenylyl]methyl}-2-methyl-1-piperazinecarboxylate (Intermediate 30, 60 mg, 0.145 mmol) in 5 mL of DCM, was added benzoyl chloride (55 mg, 0.16 mmol), followed by addition of TEA (0.05 mL, 0.3 mmol). The reaction mixture was stirred at room temperature for 1 h, and quenched by addition of 0.5 mL of saturated Na₂CO₃. The organic layer was isolated via a hydrophobic frit followed by addition of 0.5 mL of TFA. The mixture was stirred at room temperature for 1 h. After removal of the solvent, the residue was purified by Gilson reverse phase HPLC, eluting with acetonitrile/water/0.1% TFA (10/90 to 70/30, v/v, over 12 min), to give the title compound (16 mg, 12%). LC/MS: m/z, 417 (M+H), 1.58 min.

### Example 301

### 3-[(3,4-dichlorophenyl)carbonyl]-N-[(6-fluoro-3′-{[(3s)-3-methyl-1-piperazinyl]methyl}-3-biphenylyl)methyl]benzamide

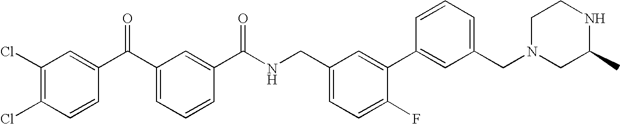

To a solution of [(3,4-dichlorophenyl)carbonyl]benzoic acid (36 mg, 0.121 mmol in CHCl₃ (2.0 mL) were added 1,1-dimethylethyl (2S)-4-({6-[3-(aminomethyl)phenyl]-2-pyridinyl}methyl)-2-methyl-1-piperazinecarboxylate (Intermediate 30, 50 mg, 0.13 mmol), TEA (0.04 ml, 0.3 mmol), EDC (36 mg, 0.19 mmol) and HOBt (18 mg, 0.14 mmol). The reaction mixture was stirred at room temperature for 2 h, then 0.5 mL of a saturated Na₂CO₃ solution was added. The organic layer was isolated via a hydrophobic frit followed by addition of 0.5 mL of TFA, and stirred at room temperature for 1 h. After removal of the solvent, the residue was purified by Gilson reverse phase HPLC, eluting with acetonitrile/water/0.1% TFA (10/90 to 70/30, v/v, over 12 min.), to give the title compound (26 mg, 36%). LC/MS: m/z, 590 (M+H), 1.66 min.

### Example 302

### N-[(6-fluoro-3′-{[(3S)-3-methyl-1-piperazinyl]methyl}-3-biphenylyl)methyl]-3-(phenylcarbonyl)benzamide

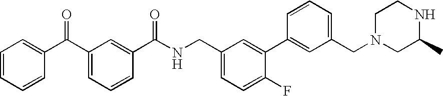

To a solution of 1,1-dimethylethyl (2S)-4-({6-[3-(aminomethyl)phenyl]-2-pyridinyl}methyl)-2-methyl-1-piperazinecarboxylate (Intermediate 30, 50 mg, 0.13 mmol) in CHCl₃ (5 mL) were added 3-(phenylcarbonyl)benzoic acid (1.5 eq), EDC (12 mg, 0.06 mmol), HOBT (1 mg, 0.006 mmol) and diisopropyl ethyl amine (0.1 mL). The resulting mixture was stirred for 12 hours and then concentrated in vacuo. Separation via a combiflash system then provided the desired amide. The amide was dissolved in CH₂Cl₂ (2 mL) and the solution was mixed with TFA (0.7 mL) at 0° C. The mixture was stirred at ambient temperature overnight, diluted with Et₃N (0.1 mL) at −78° C. and concentrated. Separation via a Gilson reverse phase HPLC then provided the title compound (60 mg, 99%). LC/MS (ES) m/z 523 (M+H)⁺; ¹HNMR (MeOD) 1.37 (3H, d), 3.05 (1H, m), 3.24 (1H, m), 3.46 (1H, m), 3.66 (4H, m), 4.37 (2H, s), 4.62 (2H, s), 7.19 (1H, t), 7.43 (1H, m), 7.56 (5H, m), 7.66 (3H, m), 7.72 (1H, s), 7.78 (2H, d), 7.93 (1H, d), 8.14 (1H, d), 8.3 (1H, s).

The compounds listed in Table 20 were prepared proceeding in a similar manner to Example 302, but replacing 3-(phenylcarbonyl)benzoic acid with the appropriate acids.

TABLE 20

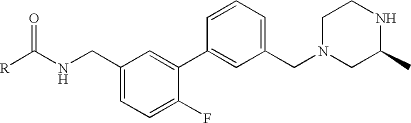

| | | MS | |
|---|---|---|---|
| Example | R | [MH]+ | NMR |
| 303 | 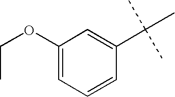 | 462 | ¹HNMR(MeOD) 1.41(6H, m), 3.13(1H, t), 3.33(1H, m), 3.45(1H, m), 3.68(4H, m), 4.07(2H, q), 4.40(2H, s), 4.60(2H, s), 7.08(1H, t), 7.19(1H, t), 7.43(4H, m), 7.53(3H, m), 7.66(1H, m), 7.73(1H, s). |
| 304 | 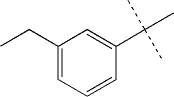 | 447 | ¹HNMR(MeOD) 1.28(3H, t), 1.39(3H, d), 2.72(2H, t), 3.08(1H, m), 3.25(1H, m), 3.46(1H, m), 3.67(4H, m), 4.36(2H, s), 4.61(2H, s), 7.20(1H, t), 7.44(3H, m), 7.55(3H, m), 7.66(2H, m), 7.72(2H, m). |

### Preparation 8

The thiophene derivatives of general structure 36 were prepared as depicted in Scheme 8. Reductive amination of the thiophene carboxaldehyde derivative 32 with the BOC-protected piperazine 26 gave the tertiary amine 33. Further palladium coupling of 33 with the commercially available boronic acid 34 produced compound 35, which in turn could be coupled with the appropriate carboxylic acids R₁CO₂H to furnish the products 36.

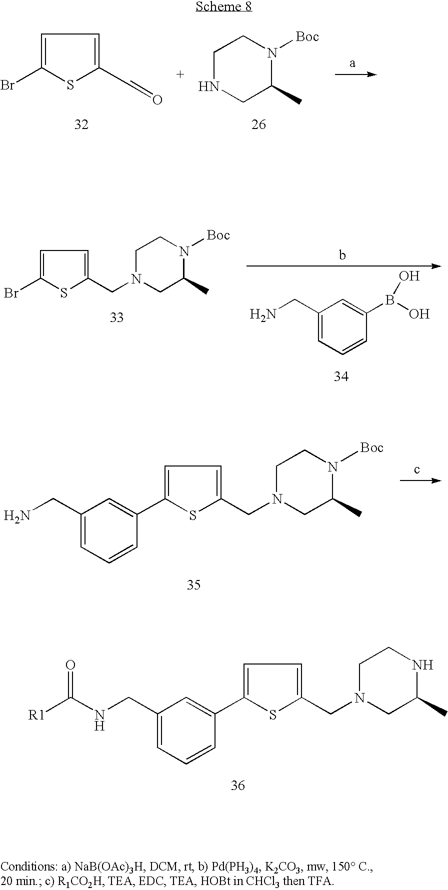

Intermediate 33

### 1,1-Dimethylethyl (2S)-4-[(5-bromo-2-thienyl)methyl]-2-methyl-1-piperazinecarboxylate

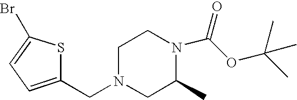

Following the standard procedure outlined for intermediate 27, 1,1-dimethylethyl (2S)-2-methyl-1-piperazinecarboxylate 26 (1.0 g, 5 mmol) was reacted with 5-bromo-2-thiophenecarbaldehyde 32 (0.96 g, 5 mmol) to give the title compound (1.43 g, 76%). LCMS: m/z, 375 (M+H), 1.63 min.

### Intermediate 35

### 1,1-dimethylethyl (2S)-4-({5-[3-(aminomethyl)phenyl]-2-thienyl}methyl)-2-methyl-1-piperazinecarboxylate

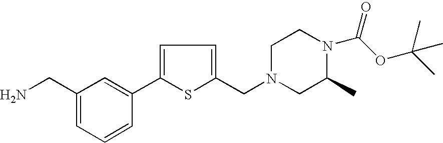

To the solution of [3-(aminomethyl)phenyl]boronic acid hydrochloride 34 (325 mg, 1.2 mmol) in dioxane/H₂O (10 mL/3.3 mL) was added (2S)-4-[(5-bromo-2-thienyl)methyl]-2-methyl-1-piperazinecarboxylate (intermediate 33, 450 mg, 1.2 mmol), K₂CO₃ (828 mg, 6.0 mmol) and Pd(PPh₃)₄ (70 mg, 0.06 mmol). The resulting solution was irradiated in a microwave reactor at 150° C. for 20 minutes and diluted with EtOAc (5 mL). The organic layer was collected and the aqueous layer was extracted by EtOAc (2×5 mL). The organic layers were combined, dried over Na₂SO₄, filtered and concentrated. The residue was purified by Gilson HPLC, eluting with acetonitrile/water/0.1% TFA (10/90 to 90/10, v/v, over 12 min), to give the title compound (200 mg, 42%). LC/MS: m/z, 402 (M+H), 1.24 min.

### Example 305

### N-{[3-(5-{[(3S)-3-methyl-1-piperazinyl]methyl}-2-thienyl)phenyl]methyl}-1,3-benzodioxole-5-carboxamide

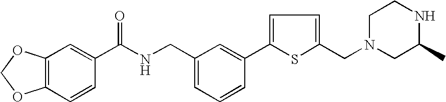

To a solution of 1,3-benzodioxole-5-carboxylic acid (12 mg, 0.075 mmol) in CHCl₃ (3.0 mL) were added 1,1-dimethylethyl (2S)-4-({5-[3-(aminomethyl)phenyl]-2-thienyl}methyl)-2-methyl-1-piperazinecarboxylate (intermediate 35, 30 mg, 0.075 mmol), TEA (0.05 ml, 0.4 mmol), EDC (22 mg, 0.113 mmol) and HOBt (11 mg, 0.083 mmol). The reaction mixture was stirred at room temperature for 15 h, followed by addition of 1 mL of saturated Na₂CO₃. The organic layer was dried over Na₂SO₄ and filtered. The filtrate was mixed with 1 mL of TFA, and stirred at room temperature for 1 h. After removal of the solvent, the residue was purified by Gilson reverse phase HPLC, eluting with acetonitrile/water/0.1% TFA (10/90 to 70/30, v/v, over 12 min), to give the title compound (31 mg, 73%). LC/MS: m/z, 350 (M+H), 1.58 min.

The compounds listed in Table 21 were prepared proceeding in a similar manner to Example 305, but replacing 1,3-benzodioxole-5-carboxylic acid with the appropriate acids.

TABLE 21

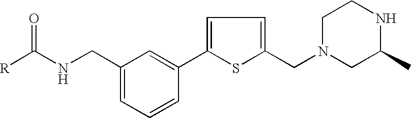

| | | MS | Rt |
|---|---|---|---|
| Example | R | [MH]+ | (min) |
| 306 | 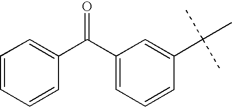 | 510 | 1.74 |
| 307 | 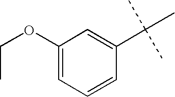 | 450 | 1.62 |
| 308 | 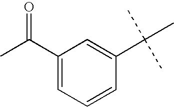 | 448 | 1.48 |

### Preparation 9

The pyridine derivatives of general structure 40 were prepared as depicted in Scheme 9. Reductive amination of the pyridine carboxaldehyde derivative 37 with the BOC-protected piperazine 26 gave the tertiary amine 38. Further palladium coupling of 38 with the commercially available boronic acid 34 produced compound 39, which in turn could be coupled with the appropriate carboxylic acid R₁CO₂H to furnish the products 40.

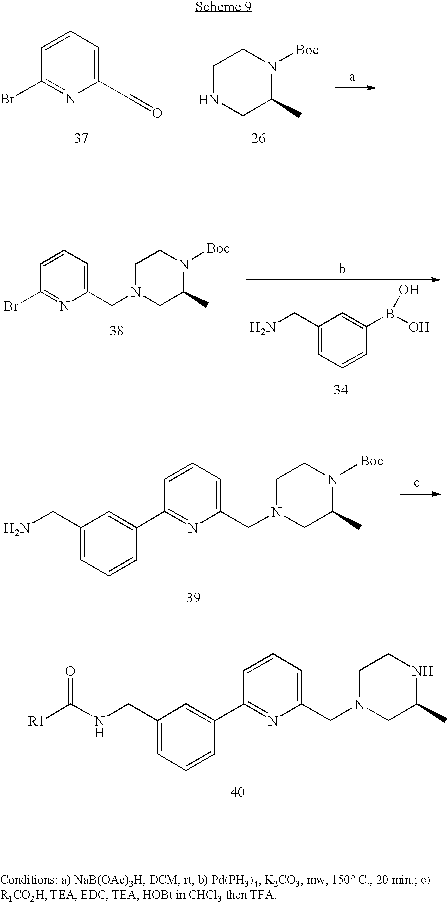

### Intermediate 38

### 1,1-dimethylethyl (2S)-4-[(6-bromo-2-pyridinyl)methyl]-2-methyl-1-piperazinecarboxylate

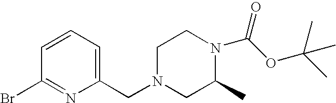

Following the standard procedure outlined for intermediate 27, 1,1-dimethylethyl (2S)-2-methyl-1-piperazinecarboxylate 26 (1.0 g, 5 mmol) was reacted with 5-bromo-2-thiophenecarbaldehyde 37 (0.96 g, 5 mmol) to give the title compound (1.43 g, 76%). LC/MS: m/z, 375 (M+H), 1.63 min.

### Intermediate 39

### 1,1-dimethylethyl (2S)-4-({6-[3-(aminomethyl)phenyl]-2-pyridinyl}methyl)-2-methyl-1-piperazinecarboxylate

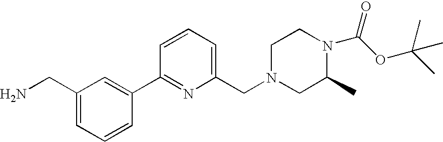

Following the standard procedure outlined for intermediate 35, 1,1-dimethylethyl (2S)-4-[(6-bromo-2-pyridinyl)methyl]-2-methyl-1-piperazinecarboxylate (Intermediate 38, 430 mg, 1.16 mmol) was reacted with [3-(aminomethyl)phenyl]boronic acid 34 (314 mg, 1.16 mmol) to give the title compound (420 mg, 92%). LC/MS: m/z, 397 (M+H), 1.22 min.

### Example 309

### 3-acetyl-N-{[3-(6-{[(3S)-3-methyl-1-piperazinyl]methyl}-2-pyridinyl)phenyl]methyl}benzamide

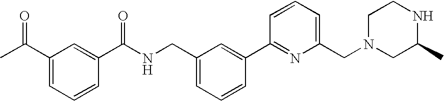

To a solution of 3-acetylbenzoic acid (21 mg, 0.13 mmol) in CHCl₃ (2.0 mL) were added 1,1-dimethylethyl (2S)-4-({6-[3-(aminomethyl)phenyl]-2-pyridinyl}methyl)-2-methyl-1-piperazinecarboxylate (intermediate 39, 50 mg, 0.13 mmol), TEA (0.04 mL, 0.3 mmol), EDC (36 mg, 0.19 mmol) and HOBt (18 mg, 0.14 mmol). The reaction mixture was stirred at room temperature for 2 h, followed by addition of 0.5 mL of saturated Na₂CO₃. The organic layer was isolated via a hydrophobic frit followed by addition of 0.5 mL of TFA, and stirred at room temperature for 1 h. After removal of the solvent, the residue was purified by Gilson reverse phase HPLC, eluting with acetonitrile/water/0.1% TFA (10/90 to 70/30, v/v, over 12 min), to give the title compound (10 mg, 10%). LC/MS: m/z, 443 (M+H), 1.20 min.

The compounds listed in Table 22 were prepared proceeding in a similar manner to Example 309, but replacing 3-acetylbenzoic acid with the appropriate acid.

TABLE 22

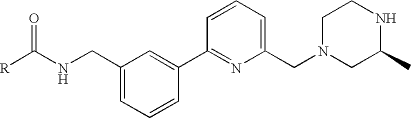

| | | MS | Rt |
|---|---|---|---|
| Example | R | [MH]+ | (min) |
| 310 | 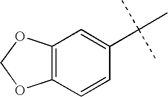 | 445 | 1.22 |
| 311 | 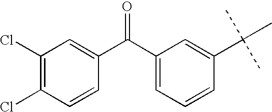 | 573 | 1.72 |
| 312 | 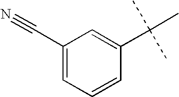 | 426 | 1.20 |
| 313 | 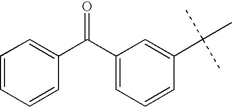 | 505 | 1.46 |
| 314 | 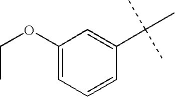 | 446 | 1.44 |

### Preparation 10

The derivatives of general structure 45 were prepared as depicted in Scheme 10. Mono-alkylation of the BOC-protected piperazine 42 with the benzyl bromide derivative 41, followed by boration of the resulting bromide with trimethyl borate under strong basic conditions gave the corresponding boronic acid 43. Further palladium coupling of 43 with 3-bromobenzonitrile, followed by reduction of the nitrile moiety produced compound 44. In turn, compound 44 could be coupled with the appropriate carboxylic acids R₁CO₂H and deprotected to furnish the products 45.

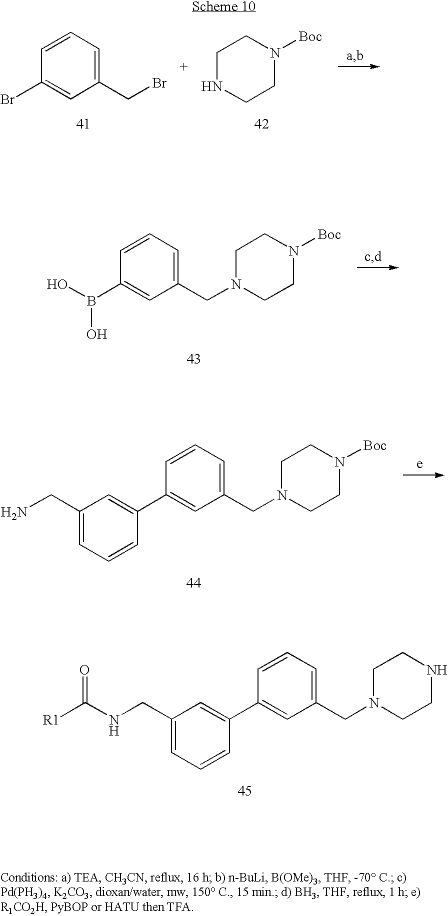

### Intermediate 40: 1,1-dimethylethyl 4-[(3-bromophenyl)methyl]-1-piperazinecarboxylate

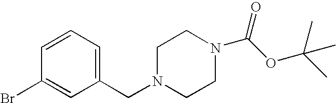

A solution of 3-bromobenzyl bromide (6 g, 24 mmol) and Boc piperazine (4.06 g, 12 mmol) in acetonitrile (30 mL) was treated with triethylamine (3.36 mL, 24 mmol). The resulting mixture was heated at reflux for 16 hours. After cooling to room temperature, the reaction mixture was treated with saturated sodium bicarbonate solution (20 mL), then extracted with ethyl acetate (2×30 mL). The organic phases were combined, dried with MgSO₄ and concentrated under vacuum. The residue was purified by chromatography on silica (100 g) eluting with ethyl acetate/cyclohexane to give the title compound (6.95 g, 81.25%). LC/MS: m/z, 355, 357 (M+H), 2.40 min.

### Intermediate 41: {3-[(4-{[(1,1-dimethylethyl)oxy]carbonyl}-1-piperazinyl)methyl]phenyl}boronic acid

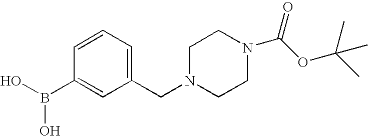

To a solution of 1,1-dimethylethyl 4-[(3-bromophenyl)methyl]-1-piperazinecarboxylate (6.55 g, 18.5 mmol) in THF (20 mL) at −70° C. was added dropwise n-butyl lithium (15.4 mL, 2.5 M solution in hexane, 38.5 mmol) over 10 minutes. After stirring for 30 mins at that temperature, the resulting orange solution was treated with trimethylborate (8.02 g, 77 mmol). The reaction mixture was then allowed to warm up to room temperature and quenched with saturated ammonium chloride (15 mL). The solvent was removed under vacuum and the residue was partitioned between ethyl acetate (20 mL) and water (20 mL). The aqueous phase was separated and further extracted with ethyl acetate (20 mL). The organic phases were combined, dried with MgSO₄ and evaporated under vacuum to give the title compound (5 g, 84%) which was used directly for the preparation of 1, 1-dimethylethyl 4-[(3′-cyano-3-biphenylyl)methyl]-1-piperazinecarboxylate without further purification. LC/MS: m/z, 321 (M+H), 1.91 min.

### Intermediate 42: 1,1-dimethylethyl 4-[(3′-cyano-3-biphenylyl)methyl]-1-piperazinecarboxylate

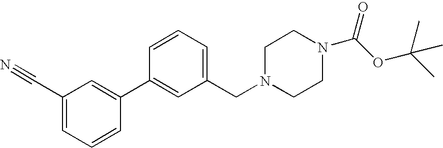

A mixture of {3-[(4-{[(1,1-dimethylethyl)oxy]carbonyl}-1-piperazinyl)methyl]phenyl}boronic acid (1 g, 3.1 mmol), 3-bromobenzonitrile (0.56 g, 3.1 mmol), potassium carbonate (1.725 g, 12.5 mmol) and tetrakis triphenylphosphine palladium (180 mg) in dioxan/water (3:1, 4 mL) was sealed in a tube and heated at 150° C. for 15 minutes in a microwave vessel. After cooling to room temperature, the reaction mixture was then diluted with water (25 mL) and extracted with ethyl acetate (2×25 mL). The combined organic phases were dried with MgSO₄ and concentrated under vacuum. The resulting crude residue was further purified by flash column chromatography on silica (100 g) to give the title compound (0.9 g, 76%) (purity ca 75%). LC/MS: m/z, 378 (M+H), 2.57 min.

### Intermediate 43

### 1,1-dimethylethyl 4-{[3′-(aminomethyl)-3-biphenylyl]methyl}-1-piperazinecarboxylate

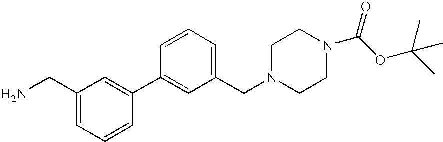

A solution of 1,1-dimethylethyl 4-[(3′-cyano-3-biphenylyl)methyl]-1-piperazinecarboxylate (4.5 g, 11.9 mmol) in THF (30 mL) was treated with borane in THF (47.7 mL, 1 M in THF, 47.7 mmol) and the resulting mixture was heated at reflux for 1 hour. After cooling to room temperature, the reaction mixture was quenched with saturated ammonium chloride solution (20 mL) and extracted with ethyl acetate (3×30 mL). The combined organics were dried (MgSO₄), concentrated under vacuum to give a residue which was purified by flash chromatography on silica (100 g) to yield the title compound (1.1 g, 24.2%). LC/MS: m/z, 382 (M+H), 1.86 min.

### Example 315

### 3-(aminosulfonyl)-N-{[3′-(1-piperazinylmethyl)-3-biphenylyl]methyl}benzamide

A mixture of PyBOP (0.08 mmol in 200 mL of DMF), {[3′-(1-piperazinylmethyl)-3-biphenylyl]methyl}amine (44 mmol in 200 mL of DMF) and DIPEA (30 mL) were added to 3-(aminosulfonyl)benzoic acid (70 mmol). The resulting mixture was stirred for 16 hours at room temperature, then the solvent was removed under vacuum. The residue was dissolved in methanol and purified by loading onto a SPE cartridge (SCX, 500 mg), washing with MeOH, and eluting with a 2M solution of NH₃ in MeOH. The NH₃ fraction was collected and evaporated under vacuum to give a gum which was dissolved in 1:1 CHCl₃/TFA (0.5 mL). After stirring for 2 hours, the solvent was removed under vacuum and the residue was dissolved in MeOH. The free base of the compound was obtained by loading the solution onto a SPE cartridge (SCX, 500 mg), washing with MeOH, and eluting with 2M NH₃/MeOH. The ammonia fraction was collected and the solvent was removed under vacuum to give the title compound (14.3 mg, 70%). LC/MS: m/z, 465 (M+H), 2.29 min.

The compounds listed in Table 23 were prepared proceeding in a similar manner to Example 315, but replacing 3-(aminosulfonyl)benzoic acid with the appropriate acids.

TABLE 23

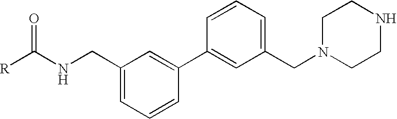

| | | MS | |
|---|---|---|---|
| Example | R | [MH]+ | Rt |
| 316 | 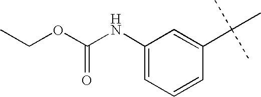 | 473 | 2.51 |
| 317 | 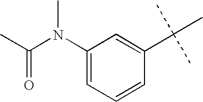 | 457 | 2.3 |
| 318 | 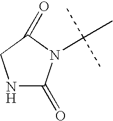 | 422 | 2.01 |
| 319 | 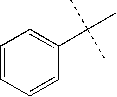 | 386 | 2.4 |
| 320 | 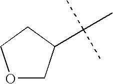 | 380 | 2.09 |
| 321 | 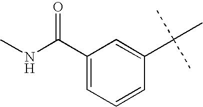 | 443 | 2.26 |
| 322 | 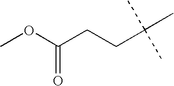 | 396 | 2.19 |
| 323 | 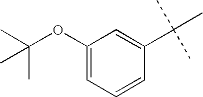 | 458 | 2.33 |
| 324 | 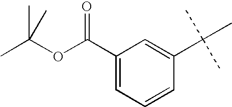 | 486 | 2.38 |
| 325 | 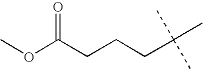 | 410 | 2.2 |
| 326 | 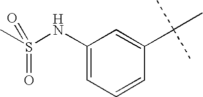 | 479 | 2.37 |
| 327 | 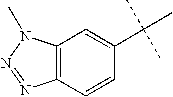 | 441 | 2.34 |
| 328 | 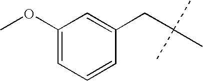 | 430 | 2.41 |
| 329 | 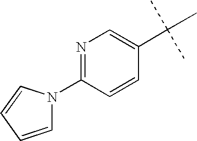 | 452 | 2.64 |
| 330 | 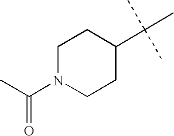 | 435 | 2.08 |
| 331 | 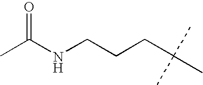 | 409 | 2.01 |
| 332 | 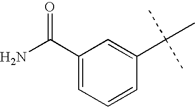 | 429 | 2.17 |
| 333 | 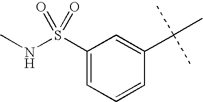 | 479 | 2.31 |
| 334 | 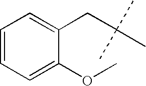 | 430 | 2.37 |
| 335 | 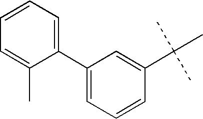 | 476 | 2.81 |
| 336 | 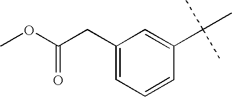 | 458 | 2.37 |
| 337 | 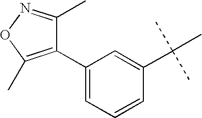 | 481 | 2.53 |
| 338 | 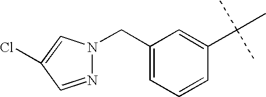 | 500 | 2.54 |
| 339 | 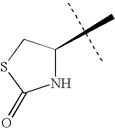 | 411 | 2.07 |
| 340 | 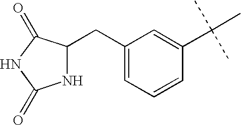 | 498 | 2.21 |
| 341 | 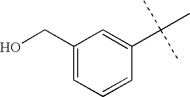 | 416 | 2.2 |
| 342 | 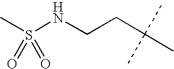 | 431 | 2.03 |
| 343 | 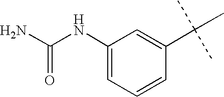 | 444 | 2.2 |
| 344 | 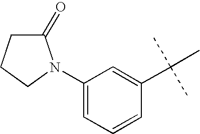 | 469 | 2.31 |

### Example 345

### 2-(4-oxo-4,5-dihydro-1,2,5-oxadiazol-3-yl)-N-{[3′-(1-piperazinylmethyl)-3-biphenylyl]methyl}acetamide trifluoroacetate

To a solution of (4-oxo-4,5-dihydro-1,2,5-oxadiazol-3-yl)acetic acid (0.1 mmol) in DMF (200 mL) was added a solution of HATU (0.1 mmol) in DMF (100 mL) followed by DIPEA (50 mL). After stirring for 10 minutes at room temperature, the mixture was treated with a solution of {[3′-(1-piperazinylmethyl)-3-biphenylyl]methyl}amine (0.075 mmol) in DMF (200 mL). After stirring for 3 days, the solvent was removed under vacuum. The residue was dissolved in methanol and purified by loading onto a SPE cartridge (SCX, 500 mg), washing with MeOH (5 mL), and eluting with a 2M solution of NH₃ in MeOH (5 mL). The solvent was removed under vacuum and the resulting gum was dissolved in 1:1 CHCl₃/TFA (0.5 mL). After stirring for 2 hours, the solvent was removed under vacuum to give a crude residue which was further purified by MDAP to afford the title compound as a TFA salt (3.8 mg, 10%). LC/MS: m/z, 408 (M+H), 2.18 min.

The compounds listed in Table 24 were prepared proceeding in a similar manner to Example 345, but replacing (4-oxo-4,5-dihydro-1,2,5-oxadiazol-3-yl)acetic acid with the appropriate acids.

TABLE 24

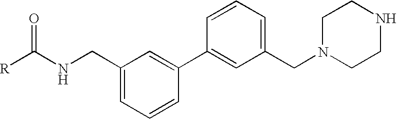

| | | MS | |
|---|---|---|---|
| Example | R | [MH]+ | Rt |
| 346 | 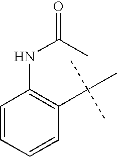 | 443 | 2.33 |
| 347 | 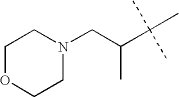 | 437 | 1.86 |
| 348 | 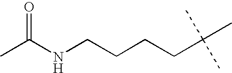 | 437 | 2.1 |

### Example 349

### N-{[3′-(1-piperazinylmethyl)-3-biphenylyl]methyl}-2-(1H-11,23-triazol-1-yl)acetamide

A mixture of PyBOP (0.08 mmol in 200 mL of DMF), {[3′-(1-piperazinylmethyl)-3-biphenylyl]methyl}amine (44 mmol in 200 mL of DMF) and DIPEA (30 mL) were added to 1H-1,2,3-triazol-1-yl acetic acid (0.07 mmol). The resulting mixture was stirred for 16 hours at room temperature, the solvent was then removed under vacuum. The residue was re-dissolved in methanol and purified by loading onto a SPE cartridge (SCX, 500 mg), washing with MeOH (5 mL), and eluting with a 2M solution of NH₃ in MeOH (5 mL). The NH₃ fraction was collected and evaporated under vacuum to give a gum which was re-dissolved in 1:1 CHCl₃/TFA (0.5 mL). After stirring for 2 hours, the solvent was removed under vacuum and the residue was purified by MDAP to give the desired compound as a TFA salt. The free base of the compound was obtained by loading the salt onto a SPE cartridge (SCX, 500 mg), washing with MeOH, and eluting with 2M NH₃/MeOH. The ammonia fraction was collected and the solvent was removed under vacuum to give the title compound (11.1 mg, 65%). LC/MS: m/z, 391 (M+H), 2.04 min.

The compounds listed in Table 25 were prepared proceeding in a similar manner to Example 349, but replacing 1H-1,2,3-triazol-1-yl acetic acid with the appropriate acids.

TABLE 25

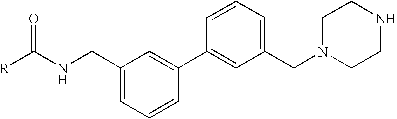

| | | MS | |
|---|---|---|---|
| Example | R | [MH]+ | Rt |
| 350 | 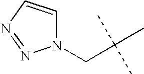 | 391 | 2.04 |
| 351 | 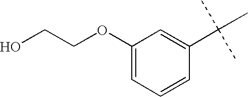 | 446 | 2.25 |
| 352 | 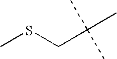 | 370 | 2.15 |
| 353 | 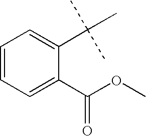 | 444 | 2.43 |
| 354 | 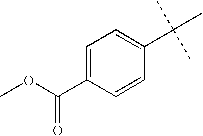 | 444 | 2.39 |
| 355 | 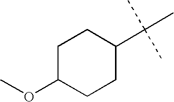 | 422 | 2.21 |
| 356 | 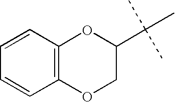 | 444 | 2.42 |
| 357 | 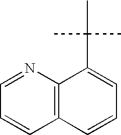 | 437 | 2.5 |
| 358 | 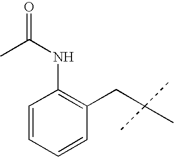 | 457 | 2.21 |
| 359 | 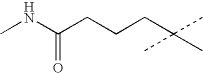 | 409 | 2.04 |

### ABBREVIATIONS

BOC tert-butyloxycarbonyl
DCM Dichlromethane
DIC 1,3-Dissopropylcarbodiimide
DIPEA Diisopropylethylamine
DMAP Dimethylaminopyridine
DME Dimethoxyethane
DMF Dimethylformamide
DMHB 2,6-dimethoxy-4-polystyrenebenzyloxy-benzaldehyde
DMSO Dimethylsulfoxide
EDCl 1-(3-Dimethylaminopropyl)-3-ethylcarbodiimide hydrochloride
EDC 1-(3-Dimethylaminopropyl)-3-ethylcarbodiimide hydrochloride
ESI Electrospray ionization
EI-MS Electrospray ionization-Mass spectrometry
HATU O-(7-Azabenzotriazol-1-yl)-N,N,N′,N′-tetramethyluronium hexafluorophosphate
HOAc Acetic acid
HOBt Hydroxybenzotriazole
HPLC High pressure liquid chromatography
LC/MS Liquid chromatography/Mass spectrometry
MDAP Mass directed automated preparative
mw Microwave
NMP 1-Methyl-2-pyrrolidinone
NMR Nuclear magnetic resonance
rt Room temperature
SPE Solid phase extraction
TEA Triethylamine
TFA Trifluoroacetic acid
THF Tetrahydrofuran

### BIOLOGICAL EXAMPLES

The inhibitory effects of compounds at the M₃ mAChR of the present invention are determined by the following in vitro and in vivo assays:

### Analysis of Inhibition of Receptor Activation by Calcium Mobilization:

### 1) 384-Well FLIPR Assay

A CHO (chinese hamster ovary) cell line stably expressing the human M3 muscarinic acetylcholine receptor is grown in DMEM plus 10% FBS, 2 mM Glutamine and 200 ug/ml G418. Cells are detached for maintenance and for plating in preparation for assays using either enzymatic or ion chelation methods. The day before the FLIPR (fluorometric imaging plate reader) assay, cells are detached, resuspended, counted, and plated to give 20,000 cells per 384 well in a 50 ul volume. The assay plates are black clear bottom plates, Becton Dickinson catalog number 35 3962. After overnight incubation of plated cells at 37 degrees C. in a tissue culture incubator, the assay is run the next day. To run the assay, media are aspirated, and cells are washed with 1× assay buffer (145 mM NaCl, 2.5 mM KCl, 10 mM glucose, 10 mM HEPES, 1.2 mM MgCl₂, 2.5 mM CaCl₂, 2.5 mM probenecid (pH 7.4.) Cells are then incubated with 50 ul of Fluo-3 dye (4 uM in assay buffer) for 60-90 minutes at 37 degrees C. The calcium-sensitive dye allows cells to exhibit an increase in fluorescence upon response to ligand via release of calcium from intracellular calcium stores. Cells are washed with assay buffer, and then resuspended in 50 ul assay buffer prior to use for experiments. Test compounds and antagonists are added in 25 ul volume, and plates are incubated at 37 degrees C. for 5-30 minutes. A second addition is then made to each well, this time with the agonist challenge, acetylcholine. It is added in 25 ul volume on the FLIPR instrument. Calcium responses are measured by changes in fluorescent units. To measure the activity of inhibitors/antagonists, acetylcholine ligand is added at an EC₈₀ concentration, and the antagonist IC₅₀ can then be determined using dose response dilution curves. The control antagonist used with M3 is atropine.

### 2) 96-Well FLIPR Assay

Stimulation of mAChRs expressed on CHO cells were analyzed by monitoring receptor-activated calcium mobilization as previously described. CHO cells stably expressing M₃ mAChRs were plated in 96 well black wall/clear bottom plates. After 18 to 24 hours, media was aspirated and replaced with 100 μl of load media (EMEM with Earl's salts, 0.1% RIA-grade BSA (Sigma, St. Louis Mo.), and 4 μM Fluo-3-acetoxymethyl ester fluorescent indicator dye (Fluo-3 AM, Molecular Probes, Eugene, Oreg.) and incubated 1 hr at 37° C. The dye-containing media was then aspirated, replaced with fresh media (without Fluo-3 AM), and cells were incubated for 10 minutes at 37° C. Cells were then washed 3 times and incubated for 10 minutes at 37° C. in 100 μl of assay buffer (0.1% gelatin (Sigma), 120 mM NaCl, 4.6 mM KCl, 1 mM KH₂ PO₄, 25 mM NaH CO₃, 1.0 mM CaCl₂, 1.1 mM MgCl₂, 11 mM glucose, 20 mM HEPES (pH 7.4)). 50 μl of compound (1×10⁻¹¹-1×10⁻⁵ M final in the assay) was added and the plates were incubated for 10 min. at 37° C. Plates were then placed into a fluorescent light intensity plate reader (FLIPR, Molecular Probes) where the dye loaded cells were exposed to excitation light (488 nm) from a 6 watt argon laser. Cells were activated by adding 50 μl of acetylcholine (0.1-10 nM final), prepared in buffer containing 0.1% BSA, at a rate of 50 μl/sec. Calcium mobilization, monitored as change in cytosolic calcium concentration, was measured as change in 566 nm emission intensity. The change in emission intensity is directly related to cytosolic calcium levels. The emitted fluorescence from all 96 wells is measured simultaneously using a cooled CCD camera. Data points are collected every second. This data was then plotting and analyzed using GraphPad PRISM software.

### Methacholine-Induced Bronchoconstriction

Airway responsiveness to methacholine was determined in awake, unrestrained BalbC mice (n=6 each group). Barometric plethysmography was used to measure enhanced pause (Penh), a unitless measure that has been shown to correlate with the changes in airway resistance that occur during bronchial challenge with methacholine. Mice were pretreated with 50 μl of compound (0.003-10 μg/mouse) in 50 μl of vehicle (10% DMSO) intranasally, and were then placed in the plethysmography chamber. Once in the chamber, the mice were allowed to equilibrate for 10 min before taking a baseline Penh measurement for 5 minutes. Mice were then challenged with an aerosol of methacholine (10 mg/ml) for 2 minutes. Penh was recorded continuously for 7 min starting at the inception of the methacholine aerosol, and continuing for 5 minutes afterward. Data for each mouse were analyzed and plotted by using GraphPad PRISM software.

The present compounds are useful for treating a variety of indications, including but not limited to respiratory-tract disorders such as chronic obstructive lung disease, chronic bronchitis, asthma, chronic respiratory obstruction, pulmonary fibrosis, pulmonary emphysema, and allergic rhinitis.

### Formulation-Administration

Accordingly, the present invention further provides a pharmaceutical formulation comprising a compound of formula (I), or a pharmaceutically acceptable salt, solvate, or physiologically functional derivative (e.g., salts and esters) thereof, and a pharmaceutically acceptable carrier or excipient, and optionally one or more other therapeutic ingredients.

Hereinafter, the term “active ingredient” means a compound of formula (I), or a pharmaceutically acceptable salt, solvate, or physiologically functional derivative thereof.

Compounds of formula (I) will be administered via inhalation via the mouth or nose.

Dry powder compositions for topical delivery to the lung by inhalation may, for example, be presented in capsules and cartridges of for example gelatine, or blisters of for example laminated aluminium foil, for use in an inhaler or insufflator. Powder blend formulations generally contain a powder mix for inhalation of the compound of the invention and a suitable powder base (carrier/diluent/excipient substance) such as mono-, di- or poly-saccharides (e.g., lactose or starch), organic or inorganic salts (e.g., calcium chloride, calcium phosphate or sodium chloride), polyalcohols (e.g., mannitol), or mixtures thereof, alternatively with one or more additional materials, such additives included in the blend formulation to improve chemical and/or physical stability or performance of the formulation, as discussed below, or mixtures thereof. Use of lactose is preferred. Each capsule or cartridge may generally contain between 20 μg-10 mg of the compound of formula (I) optionally in combination with another therapeutically active ingredient. Alternatively, the compound of the invention may be presented without excipients, or may be formed into particles comprising the compound, optionally other therapeutically active materials, and excipient materials, such as by co-precipitation or coating.

Suitably, the medicament dispenser is of a type selected from the group consisting of a reservoir dry powder inhaler (RDPI), a multi-dose dry powder inhaler (MDPI), and a metered dose inhaler (MDI).

By reservoir dry powder inhaler (RDPI) it is meant as an inhaler having a reservoir form pack suitable for comprising multiple (un-metered doses) of medicament in dry powder form and including means for metering medicament dose from the reservoir to a delivery position. The metering means may for example comprise a metering cup or perforated plate, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

The formulation can be pre-metered (eg as in Diskus, see GB 2242134 or Diskhaler, see GB 2178965, 2129691 and 2169265) or metered in use (eg as in Turbuhaler, see EP 69715). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

In one aspect, the multi-dose pack is a blister pack comprising multiple blisters for containment of medicament in dry powder form. The blisters are typically arranged in regular fashion for ease of release of medicament therefrom.

In one aspect, the multi-dose blister pack comprises plural blisters arranged in generally circular fashion on a disk-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the compound of formula (I) optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1, 1,1,2-tetrafluoroethane, 1, 1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants eg oleic acid or lecithin and cosolvents eg ethanol. Pressurized formulations will generally be retained in a canister (eg an aluminium canister) closed with a valve (eg a metering valve) and fitted into an actuator provided with a mouthpiece.

Medicaments for administration by inhalation desirably have a controlled particle size. The optimum aerodynamic particle size for inhalation into the bronchial system for localized delivery to the lung is usually 1-10 μm, preferably 2-5 μm. The optimum aerodynamic particle size for inhalation into the alveolar region for achieving systemic delivery to the lung is approximately 0.5-3 μm, preferably 1-3 μm. Particles having an aerodynamic size above 20 μm are generally too large when inhaled to reach the small airways. Average aerodynamic particle size of a formulation may measured by, for example cascade impaction. Average geometric particle size may be measured, for example by laser diffraction, optical means.

To achieve a desired particle size, the particles of the active ingredient as produced may be size reduced by conventional means eg by controlled crystallization, micronisation or nanomilling. The desired fraction may be separated out by air classification. Alternatively, particles of the desired size may be directly produced, for example by spray drying, controlling the spray drying parameters to generate particles of the desired size range. Preferably, the particles will be crystalline, although amorphous material may also be employed where desirable. When an excipient such as lactose is employed, generally, the particle size of the excipient will be much greater than the inhaled medicament within the present invention, such that the “coarse” carrier is non-respirable. When the excipient is lactose it will typically be present as milled lactose, wherein not more than 85% of lactose particles will have a MMD of 60-90 μm and not less than 15% will have a MMD of less than 15 μm. Additive materials in a dry powder blend in addition to the carrier may be either respirable, i.e., aerodynamically less than 10 microns, or non-respirable, i.e., aerodynamically greater than 10 microns.

Suitable additive materials which may be employed include amino acids, such as leucine; water soluble or water insoluble, natural or synthetic surfactants, such as lecithin (e.g., soya lecithin) and solid state fatty acids (e.g., lauric, palmitic, and stearic acids) and derivatives thereof (such as salts and esters); phosphatidylcholines; sugar esters. Additive materials may also include colorants, taste masking agents (e.g., saccharine), anti-static-agents, lubricants (see, for example, Published PCT Patent Appl. No. WO 87/905213, the teachings of which are incorporated by reference herein), chemical stabilizers, buffers, preservatives, absorption enhancers, and other materials known to those of ordinary skill.

Sustained release coating materials (e.g., stearic acid or polymers, e.g. polyvinyl pyrolidone, polylactic acid) may also be employed on active material or active material containing particles (see, for example, U.S. Pat. No. 3,634,582, GB 1,230,087, GB 1,381,872, the teachings of which are incorporated by reference herein).

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

Preferred unit dosage formulations are those containing an effective dose, as herein before recited, or an appropriate fraction thereof, of the active ingredient.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

1. A compound of formula I as indicated below: 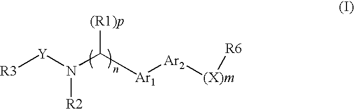
wherein
Ar1 and Ar2 are independently selected from the group consisting of optionally substituted phenyl and optionally substituted monocyclic heteroaryl;
R6 is NR₇R₈, or an optionally substituted saturated or partially unsaturated 4-10 membered ring system in which one or more rings contain one or more secondary or tertiary nitrogens, and optionally contain one or more O or S;
X is C(R1)p, or C(O); provided that when X is C(R1)p, then m is 0 or an integer of 1, 2 or 3; and when X is C(O), then m is 1;
p is 0 or is an integer of 1 or 2;
n is 0 or is an integer of 1, 2 or 3;
Y is C(O), S(O)_{q}, HNC(O), or OC(O);
q is an integer of 1 or 2;
R1 and R2 are independently selected from the group consisting of hydrogen, optionally substituted C₁-C₁₀ alkyl, optionally substituted C₃-C₁₀ cycloalkyl, optionally substituted C₃-C₁₀ cycloalkyl alkyl, optionally substituted heterocyclic, optionally substituted heterocyclicalkyl, optionally substituted C₂-C₁₀alkenyl, optionally substituted aryl, optionally substituted aryl C₁-C₁₀ alkyl, optionally substituted heteroaryl, and optionally substituted heteroaryl C₁-C₁₀alkyl;
R3 is selected from the group consisting of optionally substituted aryl, optionally substituted heteroaryl, optionally substituted C₂-C₁₀ alkenyl, optionally substituted C₁-C₁₀ alkyl, optionally substituted C₃-C₁₀ cycloalkyl, optionally substituted C₃-C₁₀ cycloalkyl alkyl, optionally substituted aryl C₁-C₁₀ alkyl, and an optionally substituted heteroaryl C₁-C₁₀alkyl moiety; and wherein these moieties are optionally substituted one or more radicals selected from the group consisting of halogen, cyano, hydroxy, hydroxy substituted C₁₋₁₀alkyl, C₁₋₁₀ alkoxy, S(O)_{m′}C₁₋₁₀ alkyl, C(O)R₄, C(O)NR₄R₅; C(O)OH; S(O)₂NR₄R₅, NHC(O)R₄, NHS(O)₂R₄, C₁₋₁₀ alkyl, C₂-C₁₀alkenyl, halosubstituted C₁₋₁₀ alkyl, optionally substituted aryl, optionally substituted aryl C₁-C₁₀ alkyl, optionally substituted heteroaryl, optionally substituted heteroaryl C₁-C₁₀alkyl, and wherein
these aryl or heteroaryl moieties may be substituted one to two times by halogen, hydroxy, hydroxy substituted alkyl, C₁₋₁₀ alkoxy, S(O)_{m′}C₁₋₁₀alkyl, C₁₋₁₀ alkyl, or halosubstituted C₁₋₁₀ alkyl; and
m′ is 0, or an integer of 1, or 2;
R₄ and R₅ are independently selected from the group consisting of hydrogen, optionally substituted C₁₋₁₀ alkyl, optionally substituted C₂-C₁₀alkenyl, optionally substituted C₃-C₁₀ cycloalkyl, optionally substituted C₃-C₁₀ cycloalkyl alkyl, optionally substituted aryl, optionally substituted aryl C₁-C₁₀ alkyl, optionally substituted heteroaryl, and optionally substituted heteroaryl C₁₋₁₀alkyl; or R₄ and R₅ together with the nitrogen to which they are attached form a 5 to 7 member ring which may optionally comprise an additional heteroatom selected from O, and S;
R₇ and R₈ are independently selected from the group consisting of hydrogen, optionally substituted C₁₋₁₀ alkyl, optionally substituted C₂-C₁₀alkenyl, optionally substituted C₃-C₁₀ cycloalkyl, optionally substituted C₃-C₁₀ cycloalkyl alkyl, optionally substituted aryl, optionally substituted aryl C₁-C₁₀alkyl, optionally substituted heteroaryl, optionally substituted heteroaryl C₁-C₁₀alkyl, optionally substituted heterocyclic, and optionally substituted heterocyclicalkyl; or R₇ and R₈ together with the nitrogen to which they are attached form a 5 to 7 member ring which may optionally comprise an additional heteroatom selected from O, N and S;
or a pharmaceutically acceptable salt thereof.

2. A. compound according to claim 1 wherein Ar1 and Ar2, are independently, selected from the group consisting of optionally substituted phenyl and optionally substituted monocyclic heteroaryl;
R6 is an optionally substituted saturated or partially unsaturated 4-10 membered ring system in which one or more rings contain one or more secondary or tertiary nitrogens;
X is C(R1)p, m is 0 or an integer of 1, 2 or 3;
p is 2;
n is an integer or 1, 2 or 3;
Y is C(O) or S(O)q; wherein q is an integer of 1 or 2;
R1 is hydrogen;
R2 is selected from the group consisting of hydrogen, optionally substituted C₁-C₁₀ alkyl, optionally substituted C₂-C₁₀ alkenyl, optionally substituted C₃-C₁₀ cycloalkyl, optionally substituted C₃-C₁₀ cycloalkyl alkyl, optionally substituted heterocyclic, optionally substituted heterocyclic alkyl, optionally substituted aryl, optionally substituted aryl C₁-C₁₀ alkyl, optionally substituted heteroaryl, and optionally substituted heteroaryl C₁-C₁₀-alkyl;
R3 is selected from the group consisting of optionally substituted aryl, optionally substituted heteroaryl, optionally substituted C₂-C₁₀ alkenyl, optionally substituted C₁-C₁₀ alkyl, optionally substituted C₃-C₁₀ cycloalkyl, and optionally substituted C₃-C₁₀ cycloalkyl alkyl moiety; and wherein
these moieties are optionally substituted by one or more radicals selected from the group consisting of halogen, cyano, hydroxy, hydroxy substituted C₁₋₁₀alkyl, C₁₋₁₀ alkoxy, S(O)_{m′}C₁₋₁₀ alkyl, C(O)R4, C(O)NR₄R₅; C(O)OH; S(O)₂NR₄R₅, NHC(O)R₄, NHS(O)₂R₄, C₁₋₁₀ alkyl, C₂₋₁₀ alkenyl, halosubstituted C₁₋₁₀ alkyl, optionally substituted aryl, optionally substituted aryl C₁₋₁₀ alkyl, optionally substituted heteroaryl, optionally substituted heteroaryl C₁₋₁₀ alkyl, and wherein
these aryl or heteroaryl moieties may be substituted one to two times by halogen, hydroxy, hydroxy substituted alkyl, C₁₋₁₀ alkoxy, S(O)_{m′}C₁₋₁₀ alkyl, C₁₋₁₀ alkyl, or halosubstituted C₁₋₁₀ alkyl; and
m′ is 0, 1, or 2;
R₄ and R₅ are independently selected from the group consisting of hydrogen, optionally substituted C₁₋₁₀ alkyl, optionally substituted C₂₋₁₀ alkenyl, optionally substituted C₃-C₁₀ cycloalkyl, optionally substituted C₃-C₁₀ cycloalkyl alkyl, optionally substituted aryl, optionally substituted aryl C₁₋₁₀alkyl, optionally substituted heteroaryl, and optionally substituted heteroaryl C₁₋₁₀ alkyl; or R₄ and R₅ together with the nitrogen to which they are attached form a 5 to 7 member ring which may optionally comprise an additional heteroatom selected from O, and S;
R₇ and R₈ are independently selected from the group consisting of hydrogen, optionally substituted C₁₋₁₀ alkyl, optionally substituted C₂₋₁₀alkenyl, optionally substituted C₃-C₁₀ cycloalkyl, optionally substituted C₃-C₁₀ cycloalkyl alkyl, optionally substituted aryl, optionally substituted aryl C₁₋₁₀alkyl, optionally substituted heteroaryl, optionally substituted heteroaryl C₁₋₁₀ alkyl, optionally substituted heterocyclic, and optionally substituted heterocyclicalkyl; or R₇ and R₈ together with the nitrogen to which they are attached form a 5 to 7 member ring which may optionally comprise an additional heteroatom selected from O, N and S;
or a pharmaceutically acceptable salt thereof.

3. A. compound according to claim 1 wherein
Ar1 and Ar2, are independently, selected from the group consisting of optionally substituted phenyl and optionally substituted monocyclic heteroaryl;
R6 is an optionally substituted saturated or partially unsaturated 5-8 membered ring system in which one or more rings contain one or more secondary or tertiary nitrogens;
X is C(R1)p;
R1 is hydrogen
p is 2;
m is 1;
n is 1;
Y is C(O), or S(O)q; wherein, q is 1 or 2;
R2 is selected from the group consisting of hydrogen, optionally substituted C₁-C₁₀ alkyl, optionally substituted alkenyl, optionally substituted C₃-C₁₀ cycloalkyl, optionally substituted C₃-C₁₀ cycloalkyl alkyl, optionally substituted heterocyclic, optionally substituted heterocyclicalkyl, optionally substituted aryl C₁-C₁₀alkyl, and optionally substituted heteroaryl C₁-C₁₀alkyl;
R3 is selected from the group consisting of optionally substituted aryl, optionally substituted heteroaryl, optionally substituted C₂-C₁₀alkenyl, optionally substituted C₁-C₁₀ alkyl, optionally substituted C₃-C₁₀ cycloalkyl, and optionally substituted C₃-C₁₀ cycloalkyl alkyl moiety; and wherein
these moieties are optionally substituted by one or more radicals selected from the group consisting of halogen, cyano, hydroxy, hydroxy substituted C₁₋₁₀alkyl, C₁₋₁₀ alkoxy, S(O)_{m′}C₁₋₁₀ alkyl, C(O)R4, C(O)NR₄R₅; C(O)OH; S(O)₂NR₄R₅, NHC(O)R₄, NHS(O)₂R₄, C₁₋₁₀ alkyl, alkenyl, and halosubstituted C₁₋₁₀ alkyl; wherein m′ is 0, 1, or 2;
R₄ and R₅, are independently, selected from the group consisting of hydrogen, optionally substituted C₁₋₁₀ alkyl, optionally substituted C₂₋₁₀alkenyl, optionally substituted C₃-C₁₀ cycloalkyl, optionally substituted C₃-C₁₀ cycloalkyl alkyl, optionally substituted aryl, optionally substituted aryl C₁₋₁₀alkyl, optionally substituted heteroaryl, and optionally substituted heteroaryl C₁₋₁₀ alkyl; or R₄ and R₅ together with the nitrogen to which they are attached form a 5 to 7 member ring which may optionally comprise an additional heteroatom selected from O, and S;
R₇ and R₈, are independently, selected from the group consisting of hydrogen, optionally substituted C₁₋₁₀ alkyl, optionally substituted C₂₋₁₀alkenyl, optionally substituted C₃-C₁₀ cycloalkyl, optionally substituted C₃-C₁₀ cycloalkyl alkyl, optionally substituted aryl, optionally substituted aryl C₁₋₁₀alkyl, optionally substituted heteroaryl, optionally substituted heteroaryl C₁₋₁₀ alkyl, optionally substituted heterocyclic, and optionally substituted heterocyclicalkyl; or R₇ and R₈ together with the nitrogen to which they are attached form a 5 to 7 member ring which may optionally comprise an additional heteroatom selected from O, N and S;
or a pharmaceutically acceptable salt thereof.

4. A. compound according to claim 1 which is
N-[(6-fluoro-3′-{[(3S)-3-methyl-1-piperazinyl]methyl}-3-biphenylyl)methyl]-3-oxo-2,3-dihydro-1H-indene-5-carboxamide;
N-[(6-fluoro-3′-{[(3S)-3-methyl-1-piperazinyl]methyl}-3-biphenylyl)methyl]-3-propanoylbenzamide;
3-acetyl-N-[(6-fluoro-3′-{[(3S)-3-methyl-1-piperazinyl]methyl}-3-biphenylyl)methyl]benzamide;
N-[(6-fluoro-3′-{[(3S)-3-methyl-1-piperazinyl]methyl}-3-biphenylyl)methyl]-3-(2-oxopropyl)benzamide;
3-(ethyloxy)-N-({6-fluoro-3′-[(3-methyl-1-piperazinyl)methyl]-3-biphenylyl}methyl)benzamide;
3-acetyl-N-[(3′-{[(3S)-3-methyl-1-piperazinyl]methyl}-3-biphenylyl)methyl]benzamide;
3-cyano-N-[(6-fluoro-3′-{[(3S)-3-methyl-1-piperazinyl]methyl}-3-biphenylyl)methyl]benzamide;
3-acetyl-N-[(6-(methyloxy)-3′-{[(3S)-3-methyl-1-piperazinyl]methyl}-3-biphenylyl)methyl]benzamide;
3-cyano-N-[(3′-{[(3S)-3-methyl-1-piperazinyl]methyl}-3-biphenylyl)methyl]benzamide;
3-(ethyloxy)-N-({6-(methyloxy)-3′-[(3-methyl-1-piperazinyl)methyl]-3-biphenylyl}methyl)benzamide;
3-acetyl-N-({3′-[(3-methyl-1-piperazinyl)methyl]-3-biphenylyl}methyl)benzamide;
3-acetyl-N-[(3′-{[(3R)-3-methyl-1-piperazinyl]methyl}-3-biphenylyl)methyl]benzamide;
methyl 3-{[({3′-[(3-methyl-1-piperazinyl)methyl]-3-biphenylyl}methyl)amino]carbonyl}benzoate;
3-cyano-N-({3′-[(1S,4S)-2,5-diazabicyclo[2.2.1]hept-2-ylmethyl]-6-fluoro-3-biphenylyl}methyl)benzamide;
3-cyano-N-({3′-[(3-methyl-1-piperazinyl)methyl]-3-biphenylyl}methyl)benzamide;
3-acetyl-N-{[3′-(1-piperazinyl methyl)-3-biphenylyl]methyl}benzamide;
3-cyano-N-[(6-(methyloxy)-3′-{[(3S)-3-methyl-1-piperazinyl]methyl}-3-biphenylyl)methyl]benzamide;
3-(ethyloxy)-N-{[3′-(1-piperazinyl methyl)-3-biphenylyl]methyl}benzamide;
3-cyano-N-{[6-fluoro-4′-(methyloxy)-3′-(1-piperazinylmethyl)-3-biphenylyl]methyl}benzamide;
3-(ethyloxy)-N-({3′-[(3-methyl-1-piperazinyl)methyl]-3-biphenylyl}methyl)benzamide;
3-acetyl-N-({3′-[(1S,4S)-2,5-diazabicyclo[2.2.1]hept-2-ylmethyl]-3-biphenylyl}methyl)benzamide;
3-(methyloxy)-N-({3′-[(3-methyl-1-piperazinyl)methyl]-3-biphenylyl}methyl)benzamide;
3-cyano-N-{[4′,6-difluoro-3′-(1-piperazinylmethyl)-3-biphenylyl]methyl}benzamide;
3-cyano-N-{[6-(methyloxy)-3′-(1-piperazinylmethyl)-3-biphenylyl]methyl}benzamide;
methyl 3-[({[3′-(1-piperazinylmethyl)-3-biphenylyl]methyl}amino)carbonyl]benzoate;
3-(methylsulfonyl)-N-{[3′-(1-piperazinylmethyl)-3-biphenylyl]methyl}benzamide;
N-[3-(4-methyl-1-piperazinyl)propyl]-N-{[3′-(1-piperazinylmethyl)-3-biphenylyl]methyl}octanamide;
methyl 3-{[({3′-[(1S,4S)-2,5-diazabicyclo[2.2.1]hept-2-ylmethyl]-3-biphenylyl}methyl)amino]carbonyl}benzoate;
3-cyano-N-{[3′-[(1S,4S)-2,5-diazabicyclo[2.2.1]hept-2-ylmethyl]-6-(methyloxy)-3-biphenylyl]methyl}benzamide;
N-({3′-[(1S,4S)-2,5-diazabicyclo[2.2.1]hept-2-ylmethyl]-3-biphenylyl}methyl)-3-(ethyloxy)benzamide;
N-([3′-[(3-methyl-1-piperazinyl)methyl]-3-biphenylyl]methyl)-3-(methylsulfonyl)benzamide;
3-cyano-N-{[3′-(1-piperazinylmethyl)-3-biphenylyl]methyl}benzamide;
N-{[3′-(1-piperazinylmethyl)-3-biphenylyl]methyl}-3-(trifluoromethyl)benzamide;
3-(methyloxy)-N-{[3′-(1-piperazinylmethyl)-3-biphenylyl]methyl}benzamide;
3-cyano-N-[(3′-{[(3R)-3-methyl-1-piperazinyl]methyl}-3-biphenylyl)methyl]benzamide;
N-({3′-[(1S,4S)-2,35-diazabicyclo[2.2.1]hept-2-ylmethyl]-3-biphenylyl}methyl)-3-(methylsulfonyl)benzamide;
3-chloro-N-{[3′-(1-piperazinylmethyl)-3-biphenylyl]methyl}benzamide;
(E)-2-phenyl-N-{[3′-(1-piperazinylmethyl)-3-biphenylyl]methyl}ethenesulfonamide;
3-cyano-N-({3′-[(1R,4R)-2,5-diazabicyclo [2.2.1]hept-2-ylmethyl]-3-biphenylyl}methyl)benzamide;
N-({3′-[(1S,4S)-2,5-diazabicyclo[2.2.1]hept-2-ylmethyl]-3-biphenylyl}methyl)-3-(methyloxy)benzamide;
4-(methyloxy)-N-{[3′-(1-piperazinylmethyl)-3-biphenylyl]methyl}benzenesulfonamide;
3-cyano-N-({3′-[(2,5-dimethyl-1-piperazinyl)methyl]-3-biphenylyl}methyl)benzamide;
N-({3′-[(3-pyrrolidinylamino)methyl]-3-biphenylyl}methyl)-1,3-benzodioxole-5-carboxamide;
N-[(6-fluoro-3′-{[(3S)-3-methyl-1-piperazinyl]methyl}-3-biphenylyl)methyl]-3-(phenylcarbonyl)benzamide;
3-(ethyloxy)-N-[(6-fluoro-3′-{[(3S)-3-methyl-1-piperazinyl]methyl}-3-biphenylyl)methyl]benzamide;
3-acetyl-N-[(4-fluoro-3′-{[(3S)-3-methyl-1-piperazinyl]methyl}-3-biphenylyl)methyl]benzamide;
3-(ethyloxy)-N-[(4-fluoro-3′-{[(3S)-3-methyl-1-piperazinyl]methyl}-3-biphenylyl)methyl]benzamide;
N-[(4-fluoro-3′-{[(3S)-3-methyl-1-piperazinyl]methyl}-3-biphenylyl)methyl]-3-(phenylcarbonyl)benzamide;
3-(2-oxo-1-pyrrolidinyl)-N-{[3′-(1-piperazinylmethyl)-3-biphenylyl]methyl}benzamide;
methyl 2-[({[3′-(1-piperazinyl methyl)-3-biphenylyl]methyl}-amino)carbonyl]benzoate;
3-[(4-chloro-1H-pyrazol-1-yl)methyl]-N-{[3′-(1-piperazinylmethyl)-3-biphenylyl]methyl}benzamide;
3-[(2-hydroxyethyl)oxy]-N-{[3′-(1-piperazinylmethyl)-3-biphenylyl]methyl}benzamide;
3-[acetyl(methyl)amino]-N-{[3′-(1-piperazinylmethyl)-3-biphenylyl]methyl}benzamide;
3-[(3,4-dichlorophenyl)carbonyl]-N-[(6-fluoro-3′-{[(3S)-3-methyl-1-piperazinyl]methyl}-3-biphenylyl)methyl]benzamide;
3-ethyl-N-[(6-fluoro-3′-{[(3S)-3-methyl-1-piperazinyl]methyl}-3-biphenylyl)methyl]benzamide;
N-[(6-fluoro-3′-{[(3S)-3-methyl-1-piperazinyl]methyl}-3-biphenylyl)methyl]benzamide;
3-[(2,5-dioxo-4-imidazolidinyl)methyl]-N-{[3′-(1-piperazinylmethyl)-3-biphenylyl]methyl}benzamide;
methyl {3-[({[3′-(1-piperazinylmethyl)-3-biphenylyl]methyl}amino)carbonyl]phenyl}acetate;
3-(3-amino-4,5-dihydro-1H-pyrazol-1-yl)-N-{[3′-(1-piperazinylmethyl)-3-biphenylyl]methyl}benzamide;
2′-methyl-N-{[3′-(1-piperazinylmethyl)-3-biphenylyl]methyl}-3-biphenylcarboxamide;
3-[(methylamino)sulfonyl]-N-{[3′-(1-piperazinylmethyl)-3-biphenylyl]methyl}benzamide;
N-methyl-N′-{[3′-(1-piperazinylmethyl)-3-biphenylyl]methyl}-1,3-benzenedicarboxamide;
3-(3,5-dimethyl-4-isoxazolyl)-N-{[3′-(1-piperazinyl methyl)-3-biphenylyl]methyl}benzamide;
3-[(methylsulfonyl)amino]-N-{[3′-(1-piperazinylmethyl)-3-biphenylyl]methyl}benzamide;
3-cyano-N-[(4-fluoro-3′-{[(3S)-3-methyl-1-piperazinyl]methyl}-3-biphenylyl)methyl]benzamide;
3-acetyl-N-{[3-(6-{[(3S)-3-methyl-1-piperazinyl]methyl}-2-pyridinyl)phenyl]methyl}benzamide;
N-{[3-(6-{[(3S)-3-methyl-1-piperazinyl]methyl}-2-pyridinyl)phenyl]methyl}-3-(phenylcarbonyl)benzamide;
3-acetyl-N-{[3-(5-{[(3S)-3-methyl-1-piperazinyl]methyl}-2-thienyl)phenyl]methyl}benzamide;
3-(hydroxymethyl)-N-{[3′-(1-piperazinyl methyl)-3-biphenylyl]methyl}benzamide;
3-(ethyloxy)-N-{[3-(6-{[(3S)-3-methyl-1-piperazinyl]methyl}-2-pyridinyl)phenyl]methyl}benzamide;
N-{[3′-(1-piperazinylmethyl)-3-biphenylyl]methyl}-1,3-benzenedicarboxamide;
N-{[3′-(1-piperazinylmethyl)-3-biphenylyl]methyl}-8-quinolinecarboxamide;
3-(aminosulfonyl)-N-{[3′-(1-piperazinylmethyl)-3-biphenylyl]-methyl}benzamide;
3-[(3,4-dichlorophenyl)carbonyl]-N-{[3-(6-{[(3S)-3-methyl-1-piperazinyl]methyl}-2-pyridinyl)phenyl]methyl}benzamide;
N-{[3′-(1-piperazinylmethyl)-3-biphenylyl]methyl}-6-(1H-pyrrol-1-yl)-3-pyridinecarboxamide; and
3-[(aminocarbonyl)amino]-N-{[3′-(1-piperazinylmethyl)-3-biphenylyl]methyl}benzamide;
or a pharmaceutically acceptable salt thereof.

5. (canceled)

6. A pharmaceutical composition comprising a compound according to claim 1 and a pharmaceutically acceptable carrier thereof.

7. A method of inhibiting the binding of acetylcholine to its receptors in a mammal in need thereof comprising administering to said mammal an effective amount of a compound according to claim 1.

8. A method of treating a muscarinic acetylcholine receptor mediated disease wherein acetylcholine binds to said receptor, in a mammal in need thereof, comprising administering to said mammal an effective amount of a compound according to claim 1.

9. A method according to claim 8 wherein the disease is selected from the group consisting of chronic obstructive lung disease, chronic bronchitis, asthma, chronic respiratory obstruction, pulmonary fibrosis, pulmonary emphysema and allergic rhinitis.

10. A method according to claim 9 wherein administration is via inhalation via the mouth or nose.

11. A method according to claim 10 wherein administration is via a medicament dispenser selected from a reservoir dry powder inhaler, a multi-dose dry powder inhaler or a metered dose inhaler.

12-14. (canceled)

15. A compound according to claim 1 which is:
N-({3′-[(1S,4S)-2,5-diazabicyclo[2.2.1]hept-2-ylmethyl]-6-fluoro-3-biphenylyl}methyl)-1,3-benzodioxole-5-carboxamide bis(trifluoroacetate);
N-[(3′-{[(3S)-3-methyl-1-piperazinyl]methyl}-3-biphenylyl)methyl]-1,3-benzodioxole-5-carboxamide bis(trifluoroacetate);
N-{[6-fluoro-3′-(1-piperazinylmethyl)-3-biphenylyl]methyl}-1,3-benzodioxole-5-carboxamide bis(trifluoroacetate);
N-({3′-[(3-methyl-1-piperazinyl)methyl]-3-biphenylyl}methyl)-1,3-benzodioxole-5-carboxamide bis(trifluoroacetate);
N-{[6-fluoro-4′-(methyloxy)-3′-(1-piperazinyl methyl)-3-biphenylyl]methyl}-1,3-benzodioxole-5-carboxamide bis(trifluoroacetate);
N-({3′-[(3-methyl-1-piperazinyl)methyl]-3-biphenylyl}methyl)-2,1,3-benzoxad iazole-5-carboxamide bis(trifluoroacetate);
N-{[3′-(1-piperazinylmethyl)-3-biphenylyl]methyl}-2,1,3-benzoxad iazole-5-carboxamide bis(trifluoroacetate);
N-{[4′,6-difluoro-3′-(1-piperazinylmethyl)-3-biphenylyl]methyl}-1,3-benzodioxole-5-carboxamide bis(trifluoroacetate);
N-{[3′-(1-piperazinylmethyl)-3-biphenylyl]methyl}-1,3-benzodioxole-5-carboxamide bis(trifluoroacetate);
N-{[6-(methyloxy)-3′-(1-piperazinylmethyl)-3-biphenylyl]methyl}-1,3-benzodioxole-5-carboxamide bis(trifluoroacetate);
N-({3′-[(1S,4S)-2,5-diazabicyclo[2.2.1]hept-2-ylmethyl]-3-biphenylyl}methyl)-2,1,3-benzoxadiazole-5-carboxamide bis(trifluoroacetate);
N-{[3′-[(1S,4S)-2,5-diazabicyclo[2.2.1]hept-2-ylmethyl]-6-(methyloxy)-3-biphenylyl]methyl}-1,3-benzodioxole-5-carboxamide bis(trifluoroacetate);
N-[(3′-{[(3R)-3-methyl-1-piperazinyl]methyl}-3-biphenylyl)methyl]-1,3-benzodioxole-5-carboxamide bis(trifluoroacetate);
N-({3′-[(1S,4S)-2,5-diazabicyclo[2.2.1]hept-2-ylmethyl]-3-biphenylyl}methyl)-1,3-benzodioxole-5-carboxamide bis(trifluoroacetate);
N-{[3′-(hexahydro-1H-1,4-diazepin-1-ylmethyl)-3-biphenylyl]methyl}-1,3-benzodioxole-5-carboxamide bis(trifluoroacetate);
N-({3′-[(4-acetyl-1-piperazinyl)methyl]-3-biphenylyl}methyl)-1,3-benzodioxole-5-carboxamide trifluoroacetate;
N-({3′-[(2,5-dimethyl-1-piperazinyl)methyl]-3-biphenylyl}methyl)-1,3-benzodioxole-5-carboxamide bis(trifluoroacetate);
N-({3′-[(3-amino-1-pyrrolidinyl)methyl]-3-biphenylyl}methyl)-1,3-benzodioxole-5-carboxamide bis(trifluoroacetate);
N-({3′-[(4-methyl-1-piperazinyl)methyl]-3-biphenylyl}methyl)-1,3-benzodioxole-5-carboxamide bis(trifluoroacetate);
N-[(4-fluoro-3′-{[(3S)-3-methyl-1-piperazinyl]methyl}-3-biphenylyl)methyl]-1,3-benzodioxole-5-carboxamide;
1-methyl-N-{[3′-(1-piperazinylmethyl)-3-biphenylyl]methyl}-1H-1,2,3-benzotriazole-6-carboxamide;
N-{[3′-(1-piperazinyl methyl)-3-biphenylyl]methyl}-2-(3-pyridinyl)-1,3-thiazole-4-carboxamide;
N-{[3-(6-{[(3S)-3-methyl-1-piperazinyl]methyl}-2-pyridinyl)phenyl]methyl}-1,3-benzodioxole-5-carboxamide; or
a pharmaceutically acceptable salt thereof.

16. A compound of the formula 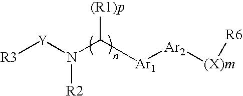
wherein
Ar1 and Ar2 are independently selected from an optionally substituted phenyl;
R6 is an optionally substituted saturated or partially unsaturated 5-8 membered ring system in which one or more rings contain one or more secondary or tertiary nitrogens;
X is C(R1)ₚ,
m is 1;
p is 1;
n is 1;
Y is C(O);
R1 is hydrogen;
R2 is hydrogen, optionally substituted C₁-C₁₀ alkyl, optionally substituted C₃-C₁₀ cycloalkyl, optionally substituted C₃-C₁₀ cycloalkyl alkyl, optionally substituted heterocyclic, optionally substituted heterocyclicalkyl, optionally substituted C₂-C₁₀alkenyl, optionally substituted aryl, optionally substituted aryl C₁-C₁₀alkyl, optionally substituted heteroaryl, or optionally substituted heteroaryl C₁-C₁₀alkyl;
R3 is an optionally substituted aryl, optionally substituted heteroaryl, optionally substituted C₂₋₁₀alkenyl, optionally substituted C₁-C₁₀ alkyl, optionally substituted C₃-C₁₀ cycloalkyl, optionally substituted C₃-C₁₀ cycloalkyl alkyl, optionally substituted aryl C₁₋₁₀ alkyl, or a optionally substituted heteroaryl C₁₋₁₀ alkyl moiety; and
wherein these moieties are optionally substituted one or more times by halogen, cyano, hydroxy, hydroxy substituted C₁₋₁₀ alkyl, C₁₋₁₀ alkoxy, S(O)_{m′}C₁₋₁₀ alkyl, C(O)R4, C(O)NR₄R₅; C(O)OH; S(O)₂NR₄R₅, NHC(O)R₄, NHS(O)₂R₄, C₁₋₁₀ alkyl, C₂₋₁₀alkenyl, halosubstituted C₁₋₁₀ alkyl, optionally substituted aryl, optionally substituted aryl C₁₋₁₀alkyl, optionally substituted heteroaryl, optionally substituted heteroaryl C₁₋₁₀ alkyl, and wherein the aryl or heteroaryl moieties may be substituted one to two times by halogen, hydroxy, hydroxy substituted alkyl, C₁₋₁₀ alkoxy, S(O)_{m′}C₁₋₁₀ alkyl, C₁₋₁₀ alkyl, or halosubstituted C₁₋₁₀ alkyl; and m′ is 0, 1, or 2;
R₄ and R₅ are independently selected from the group consisting of hydrogen, optionally substituted C₁₋₁₀ alkyl, optionally substituted C₂₋₁₀alkenyl, optionally substituted C₃-C₁₀ cycloalkyl, optionally substituted C₃-C₁₀ cycloalkyl alkyl, optionally substituted aryl, optionally substituted aryl C₁₋₁₀alkyl, optionally substituted heteroaryl, and optionally substituted heteroaryl C₁₋₁₀alkyl; or R₄ and R₅ together with the nitrogen to which they are attached form a 5 to 7 member ring which may optionally comprise an additional heteroatom selected from O, and S;
or a pharmaceutically acceptable salt thereof.

17. The compound according to claim 16 wherein the configuration of Ar1 and Ar2 is:

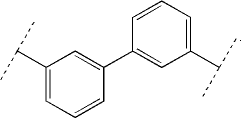

18. The compound according to claim 16 wherein R6 is piperazinyl, pyrrolidinyl, piperidinyl, hexahydroazepinyl, 4-methyl-hexahydro-1,4-diazepinyl, 4-methylpiperazinyl, hexahydro-1,4-diazepinyl, 4-acetyl-piperazinyl. 4-ethyl-piperazinyl, 3-amino-pyrrolidinyl, 3-amino-piperadinyl, 4-formyl-piperidinyl, (1s,4s)-2,5-diazobicyclo[2.21]hetp-2-yl, 1-azabicyclo[2.2.1]oct-3-yl, or 3-methyl-piperazinyl, 3,5-dimethylpiperazinyl.

19. The compound according to claim 17 wherein R6 is piperazinyl, pyrrolidinyl, piperidinyl, hexahydroazepinyl, 4-methyl-hexahydro-1,4-diazepinyl, 4-methylpiperazinyl, hexahydro-1,4-diazepinyl, 4-acetyl-piperazinyl. 4-ethyl-piperazinyl, 3-amino-pyrrolidinyl, 3-amino-piperadinyl, 4-formyl-piperidinyl, (1s,4s)-2,5-diazobicyclo[2.21]hetp-2yl, 1-azabicyclo[2.2.1]oct-3-yl, or 3-methyl-piperazinyl, 3,5-dimethylpiperazinyl.

20. The compound according to claim 16 wherein R3 is an optionally substituted aryl.

21. The compound according to claim 20 wherein R3 is a phenyl, substituted by C(O)R₄, C(O)NR₄R₅, C(O)OH, or NHC(O)R₄.

22. The compound according to claim 21 wherein R2 is hydrogen.

23. The compound according to claim 17 wherein R2 is hydrogen.

24. The compound which is: [chemical structure]
